(19) United States
(12) Patent Application Publication
(10) Pub. No.: US 2007/0093455 A1
(43) Pub. Date: Apr. 26, 2007

(54) 10-substituted tetracyclines and methods of use thereof

(76) Inventors: Paul Abato, Providence, RI (US); Haregewein Assefa, Braintree, MA (US); Joel Berniac, Stoneham, MA (US); Todd Bowser, Charlton, MA (US); Jackson Chen, East Amherst, NY (US); Mark Grier, Watertown, MA (US); Laura Honeyman, Roslindale, MA (US)

Correspondence Address: LAHIVE & COCKFIELD, LLP, ONE POST OFFICE SQUARE, BOSTON, MA, 02109-2127, US

(21) Appl. No.: 11/490,867

(22) Filed: Jul. 21, 2006

Related U.S. Application Data
(60) Provisional application No. 60/701,730, filed on Jul. 21, 2005.
(60) Provisional application No. 60/816,066, filed on Jun. 23, 2006.

(51) Int. Cl. A61K 31/65; C07C 237/26

(52) U.S. Cl. 514/114; 514/152; 546/61; 549/23; 549/384; 552/204

(57) ABSTRACT

10-Substituted tetracycline compounds are described.

### RELATED APPLICATION

This application is claims priority to U.S. Provisional Patent Application Ser. No. 60/816,066, filed on Jun. 23, 2006 and to U.S. Provisional Patent Application Ser. No 60/701,730, entitled “10 Substituted Tetracyclines and Methods of Use Thereof,” filed on July 21, 2005; the entire contents of each of which are hereby incorporated herein by reference.

### BACKGROUND OF THE INVENTION

The development of the tetracycline antibiotics was the direct result of a systematic screening of soil specimens collected from many parts of the world for evidence of microorganisms capable of producing bacteriocidal and/or bacteriostatic compositions. The first of these novel compounds was introduced in 1948 under the name chlortetracycline. Two years later, oxytetracycline became available. The elucidation of the chemical structure of these compounds confirmed their similarity and furnished the analytical basis for the production of a third member of this group in 1952, tetracycline. A new family of tetracycline compounds, without the ring-attached methyl group present in earlier tetracyclines, was prepared in 1957 and became publicly available in 1967; and minocycline was in use by 1972.

Recently, research efforts have focused on developing new tetracycline antibiotic compositions effective under varying therapeutic conditions and routes of administration. New tetracycline analogues have also been investigated which may prove to be equal to or more effective than the originally introduced tetracycline compounds. Examples include U.S. Pat. Nos. 2,980,584; 2,990,331; 3,062,717; 3,165,531; 3,454,697; 3,557,280; 3,674,859; 3,957,980; 4,018,889; 4,024,272; and 4,126,680. These patents are representative of the range of pharmaceutically active tetracycline and tetracycline analogue compositions.

Historically, soon after their initial development and introduction, the tetracyclines were found to be highly effective pharmacologically against rickettsiae; a number of gram-positive and gram-negative bacteria; and the agents responsible for lymphogranuloma venereum, inclusion conjunctivitis, and psittacosis. Hence, tetracyclines became known as “broad spectrum” antibiotics. With the subsequent establishment of their in vitro antimicrobial activity, effectiveness in experimental infections, and pharmacological properties, the tetracyclines as a class rapidly became widely used for therapeutic purposes. However, this widespread use of tetracyclines for both major and minor illnesses and diseases led directly to the emergence of resistance to these antibiotics even among highly susceptible bacterial species both commensal and pathogenic (e.g., pneumococci and Salmonella). The rise of tetracycline-resistant organisms has resulted in a general decline in use of tetracyclines and tetracycline analogue compositions as antibiotics of choice.

More recently, tetracycline compounds have also been found useful against a wide variety of disorders not necessarily related to antibacterial activity. Examples of such disorders include, for example, cancer, inflammatory disorders (e.g., arthritis),viral infections, neurological disorders, aortic or vascular aneurysms, ischemia, stroke, chronic lung disorders, bone mass disorders and diabetes.

### SUMMARY OF THE INVENTION

In one embodiment, the invention pertains, at least in part, to 10-substituted tetracycline compounds. In a further embodiment, the invention pertains to compounds of formula (I): 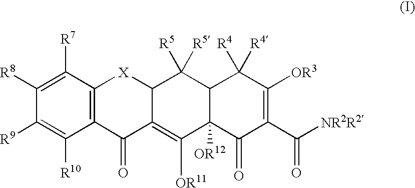
wherein
X is CHC(R¹³Y′Y), C═CR¹³Y, CR^{6′}R⁶, S, NR⁶, or O;
R^{2′}, R^{2″}, R^{4a}, and R^{4b} are each independently hydrogen, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R³, R¹¹ and R¹² are each are each independently hydrogen, alkyl, alkenyl, aryl, alkynyl, aralkyl, acetyl, alkylcarbonyl, alkenylcarbonyl, arylcarbonyl, alkynylcarbonyl, alkyloxycarbonyl, alkenyloxycarbonyl, alkynyloxycarbonyl, aryloxycarbonyl, alkylaminocarbonyl, alkenylaminocarbonyl, alkynylaminocarbonyl, arylaminocarbonyl, alkylthiocarbonyl, alkenylthiocarbonyl, alkynylthiocarbonyl, arylthiocarbonyl, alkyloxythiocarbonyl, alkenyloxythiocarbonyl, alkynyloxythiocarbonyl, aryloxythiocarbonyl, alkylaminothiocarbonyl, alkenylaminothiocarbonyl, alkynylaminothiocarbonyl, arylaminothiocarbonyl, alkylthiothiocarbonyl, alkenylthiothiocarbonyl, alkynylthiothiocarbonyl, or arylthiothiocarbonyl;
R⁴ and R^{4′} are each independently NR^{4a}R^{4b}, alkyl, alkenyl, alkynyl, hydroxyl, halogen, or hydrogen;
R⁵ and R^{5′} are each independently hydroxyl, hydrogen, thiol, alkanoyl, aroyl, alkaroyl, aryl, heteroaromatic, alkyl, alkenyl, alkynyl, alkoxy, alkylthio, alkylsulfinyl, alkylsulfonyl, alkylamino, arylalkyl, alkyl carbonyloxy, or aryl carbonyloxy;
R⁶ and R^{6′} are each independently hydrogen, methylene, absent, hydroxyl, halogen, thiol, alkyl, alkenyl, alkynyl, aryl, alkoxy, alkylthio, alkylsulfinyl, alkylsulfonyl, alkylamino, or an arylalkyl;
R⁷ is hydrogen, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino, acyl, acylamino, amidino, imino, sulfhydryl, alkylthio, arylthio, thiocarboxylate, sulfates, alkylsulfinyl, arylsulfinyl, alkylsulfonyl, arylsulfonyl, sulfonato, sulfamoyl, sulfonamido, nitro, trifluoromethyl, cyano, azido, alkylaryl, aryl, a heterocyclic moiety or —(CH₂)₀₋₃(NR^{7c})₀₋₁C(═W′)WR^{7a};
R⁸ is hydrogen, hydroxyl, halogen, thiol, nitro, alkyl, alkenyl, alkynyl, aryl, alkoxy, alkylthio, alkylsulfinyl, alkylsulfonyl, alkylamino, amino, arylalkenyl, arylalkynyl, acyl, aminoalkyl, heterocyclic, thionitroso, or —(CH₂)₀₋₃(NR^{8c})₀₋₁C(=E′)ER^{8a};
R⁹ is hydrogen, hydroxyl, halogen, thiol, nitro, alkyl, alkenyl, alkynyl, aryl, alkoxy, alkylthio, alkylsulfinyl, alkylsulfonyl, arylalkyl, amido, carboxylate, aminocarbonyl, arylalkenyl, arylalkynyl, acyl, aminoalkyl, heterocyclic, thionitroso, or —(CH₂)₀₋₃(NR^{9c})₀₋₁C(=Z′)ZR^{9a};
R¹⁰ is hydrogen, alkyl, alkenyl, alkynyl, halogen, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, arylaminocarbonyl, alkylthiocarbonyl, arylthiocarbonyl; phosphate, phosphonato, phosphinato, cyano, amino, acylamino, amidino, imino, sulfhydryl, alkylthio, arylthio, thiocarboxylate, sulfates, alkylsulfinyl, arylsulfinyl, alkylsulfonyl, arylsulfonyl, sulfonato, sulfamoyl, sulfonamido, nitro, trifluoromethyl, cyano, azido, alkylaryl, aryl or a heterocyclic moiety;
R^{7a}, R^{7b}, R^{7c}, R^{7d}, R^{7e}, R^{7f}, R^{8a}, R^{8b}, R^{8c}, R^{8d}, R^{8e}, R^{8f}, R^{9a}, R^{9b}, R^{9c}, R^{9d}, R^{9e}, and R^{9f} are each independently hydrogen, acyl, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R¹³ is hydrogen, hydroxy, alkyl, alkenyl, alkynyl, alkoxy, alkylthio, aryl, alkylsulfinyl, alkylsulfonyl, alkylamino, or an arylalkyl;
E is CR^{8d}R^{8e}, S, NR^{8b} or O;
E′ is O, NR^{8f}, for S;
W is CR^{7d}R^{7e}, S, NR^{7b} or O;
W′ is O, NR^{7f}, for S;
Y′ and Y are each independently hydrogen, halogen, hydroxyl, cyano, sulfhydryl, amino, alkyl, alkenyl, alkynyl, alkoxy, alkylthio, alkylsulfinyl, alkylsulfonyl, alkylamino, or an arylalkyl;
Z is CR^{9d}R^{9e}, S, NR^{9b} or O;
Z′ is O, S, or NR^{9f}, and pharmaceutically acceptable salts, prodrugs, esters and enantiomers thereof.

In another embodiment, the invention pertains, at least in part, to tetracycline compounds of formula (II): 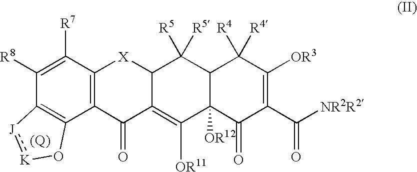
wherein
X is CHC(R¹³Y′Y), C═CR¹³Y, CR^{6′}R⁶, S, NR⁶, or O;
R^{2′}, R^{2″}, R^{4a}, and R^{4b} are each independently hydrogen, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R³, R¹¹ and R¹² are each are each independently hydrogen, alkyl, alkenyl, aryl, alkynyl, aralkyl, acetyl, alkylcarbonyl, alkenylcarbonyl, arylcarbonyl, alkynylcarbonyl, alkyloxycarbonyl, alkenyloxycarbonyl, alkynyloxycarbonyl, aryloxycarbonyl, alkylaminocarbonyl, alkenylaminocarbonyl, alkynylaminocarbonyl, arylaminocarbonyl, alkylthiocarbonyl, alkenylthiocarbonyl, alkynylthiocarbonyl, arylthiocarbonyl, alkyloxythiocarbonyl, alkenyloxythiocarbonyl, alkynyloxythiocarbonyl, aryloxythiocarbonyl, alkylaminothiocarbonyl, alkenylaminothiocarbonyl, alkynylaminothiocarbonyl, arylaminothiocarbonyl, alkylthiothiocarbonyl, alkenylthiothiocarbonyl, alkynylthiothiocarbonyl, or arylthiothiocarbonyl;
R⁴ and R^{4′} are each independently NR^{4a}R^{4b}, alkyl, alkenyl, alkynyl, hydroxyl, halogen, or hydrogen;
R⁵ and R^{5′} are each independently hydroxyl, hydrogen, thiol, alkanoyl, aroyl, alkaroyl, aryl, heteroaromatic, alkyl, alkenyl, alkynyl, alkoxy, alkylthio, alkylsulfinyl, alkylsulfonyl, alkylamino, arylalkyl, alkyl carbonyloxy, or aryl carbonyloxy;
R⁶ and R^{6′} are each independently hydrogen, methylene, absent, hydroxyl, halogen, thiol, alkyl, alkenyl, alkynyl, aryl, alkoxy, alkylthio, alkylsulfinyl, alkylsulfonyl, alkylamino, or an arylalkyl;
R⁷ is hydrogen, alkyl, alkenyl, alkynyl, halogen, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, arylaminocarbonyl, alkylthiocarbonyl, arylthiocarbonyl; phosphate, phosphonato, phosphinato, cyano, amino, acylamino, amidino, imino, sulfhydryl, alkylthio, arylthio, thiocarboxylate, sulfates, alkylsulfinyl, arylsulfinyl, alkylsulfonyl, arylsulfonyl, sulfonato, sulfamoyl, sulfonamido, nitro, trifluoromethyl, cyano, azido, alkylaryl, aryl, a heterocyclic moiety, or —(CH₂)₀₋₃(NR^{7c})₀₋₁C(═W′)WR^{7a};
R⁸ is hydrogen, hydroxyl, halogen, thiol, nitro, alkyl, alkenyl, alkynyl, aryl, alkoxy, alkylthio, alkylsulfinyl, alkylsulfonyl, alkylamino, amino, arylalkenyl, arylalkynyl, acyl, aminoalkyl, heterocyclic, thionitroso, or —(CH₂)₀₋₃(NR^{8c})₀₋₁C(=E′)ER^{8a};
R^{7a}, R^{7b}, R^{7c}, R^{7d}, R^{7e}, R^{7f}, R^{8a}, R^{8b}, R^{8c}, R^{8d}, R^{8e}, R^{8f} are each independently hydrogen, acyl, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R¹³ is hydrogen, hydroxy, alkyl, alkenyl, alkynyl, alkoxy, alkylthio, aryl, alkylsulfinyl, alkylsulfonyl, alkylamino, or an arylalkyl;
J is CR^{21a}R^{21b}, O, S, or NR^{21c};
K is CR^{22a}R^{22b}, O, S, or NR^{22c}
R^{21a}, R^{21b}, R^{21c}, R^{22a}, R^{22b}, R^{22c} are each independently hydrogen, hydroxyl, alkyl, alkenyl, alkynyl, aryl, arylalkyl, amido, alkylamino, amino, arylamino, alkylcarbonyl, arylcarbonyl, alkylaminocarbonyl, alkoxy, alkoxycarbonyl, alkylcarbonyl, alkyloxycarbonyl, arylcarbonyloxy, aryloxy, phosphate, phosphonato, phosphinato, cyano, acylamino, amidino, imino, sulfhydryl, thiol, alkylthiol, arylthiol, thiocarboxylate, sulfates, alkylsulfinyl, arylsulfinyl, alkylsulfonyl, arylsulfonyl, sulfonato, sulfamoyl, sulfonamido, nitro, trifluoromethyl, cyano, azido, heterocyclyl, alkylsilyl, arylsilyl, or absent;
Q is a double bond when J is CR^{21a}R^{21b}, K is CR^{22a}R^{22b} and R^{21b} and R^{22b} are absent;
Q is a double bond when J is NR^{21c}, K is CR^{22a}R^{22b} and R^{21c} and R^{22b} are absent;
Q is a double bond when J is CR^{21a}R^{21b}, K is NR^{22c}, and R^{21b} and R^{22c} are absent;
Q is a single bond when J is CR^{21a}R^{2b}, O, S, or NR^{21c}, K is CR^{22a}R^{22b}, O, S, or NR^{22c} and R^{21a}, R^{21b}, R^{21c}, R^{22a}, R^{22b}, R^{22c} are each independently hydrogen, hydroxyl, alkyl, alkenyl, alkynyl, aryl, arylalkyl, amido, alkylamino, amino, arylamino, alkylcarbonyl, arylcarbonyl, alkylaminocarbonyl, alkoxy, alkoxycarbonyl, alkylcarbonyl, alkyloxycarbonyl, arylcarbonyloxy, aryloxy, phosphate, phosphonato, phosphinato, cyano, acylamino, amidino, imino, sulfhydryl, thiol, alkylthiol, arylthiol, thiocarboxylate, sulfates, alkylsulfinyl, arylsulfinyl, alkylsulfonyl, arylsulfonyl, sulfonato, sulfamoyl, sulfonamido, nitro, trifluoromethyl, cyano, azido, heterocyclyl, alkylsilyl, or arylsilyl;
E is CR^{8d}R^{8e}, S, NR^{8b} or O;
E′ is ), NR^{8f}, or S;
W is CR^{7d}R^{7e}, S, NR^{7b} or O;
W′ is ), NR^{7f}, or S;
Y′ and Y are each independently hydrogen, halogen, hydroxyl, cyano, sulfhydryl, amino, alkyl, alkenyl, alkynyl, alkoxy, alkylthio, alkylsulfinyl, alkylsulfonyl, alkylamino, or an arylalkyl; and pharmaceutically acceptable salts, prodrugs, esters and enantiomers thereof.

In yet another embodiment, the invention pertains, at least in part, to tetracycline compounds of formula (III): 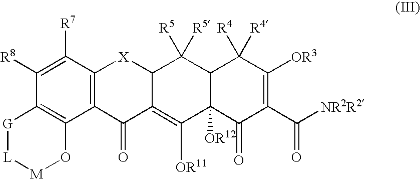
wherein
X is CHC(R¹³Y′Y), C═CR¹³Y, CR^{6′}R⁶, S, NR⁶, or O;
R^{2′}, R^{2″}, R^{4a}, and R^{4b} are each independently hydrogen, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R³, R¹¹ and R¹² are each are each independently hydrogen, alkyl, alkenyl, aryl, alkynyl, aralkyl, acetyl, alkylcarbonyl, alkenylcarbonyl, arylcarbonyl, alkynylcarbonyl, alkyloxycarbonyl, alkenyloxycarbonyl, alkynyloxycarbonyl, aryloxycarbonyl, alkylaminocarbonyl, alkenylaminocarbonyl, alkynylaminocarbonyl, arylaminocarbonyl, alkylthiocarbonyl, alkenylthiocarbonyl, alkynylthiocarbonyl, arylthiocarbonyl, alkyloxythiocarbonyl, alkenyloxythiocarbonyl, alkynyloxythiocarbonyl, aryloxythiocarbonyl, alkylaminothiocarbonyl, alkenylaminothiocarbonyl, alkynylaminothiocarbonyl, arylaninothiocarbonyl, alkylthiothiocarbonyl, alkenylthiothiocarbonyl, alkynylthiothiocarbonyl, or arylthiothiocarbonyl;
R⁴ and R^{4′} are each independently NR^{4a}R^{4b}, alkyl, alkenyl, alkynyl, hydroxyl, halogen, or hydrogen;
R⁵ and R^{5′} are each independently hydroxyl, hydrogen, thiol, alkanoyl, aroyl, alkaroyl, aryl, heteroaromatic, alkyl, alkenyl, alkynyl, alkoxy, alkylthio, alkylsulfinyl, alkylsulfonyl, alkylamino, arylalkyl, alkyl carbonyloxy, or aryl carbonyloxy;
R⁶ and R^{6′} are each independently hydrogen, methylene, absent, hydroxyl, halogen, thiol, alkyl, alkenyl, alkynyl, aryl, alkoxy, alkylthio, alkylsulfinyl, alkylsulfonyl, alkylamino, or an arylalkyl;
R⁷ is hydrogen, hydroxyl, halogen, thiol, nitro, alkyl, alkenyl, alkynyl, aryl, alkoxy, alkylthio, alkylsulfinyl, alkylsulfonyl, arylalkyl, amino, arylalkenyl, arylalkynyl, acyl, aminoalkyl, heterocyclic, thionitroso, or —(CH₂)₀₋₃(NR^{7c})₀₁C(═W′)WR^{7a};
R⁸ is hydrogen, hydroxyl, halogen, thiol, nitro, alkyl, alkenyl, alkynyl, aryl, alkoxy, alkylthio, alkylsulfinyl, alkylsulfonyl, alkylamino, amino, arylalkenyl, arylalkynyl, acyl, aminoalkyl, heterocyclic, thionitroso, or —(CH₂)₀₋₃(NR^{8c})₀₋₁C(=E′)ER^{8a};
R^{7a}, R^{7b}, R^{7c}, R^{7d}, R^{7e}, R^{7f}, R^{8a}, R^{8b}, R^{8c}, R^{8b}, R^{8c}, R^{8d}, R^{8e}, R^{8f} are each independently hydrogen, acyl, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R¹³ is hydrogen, hydroxy, alkyl, alkenyl, alkynyl, alkoxy, alkylthio, aryl, alkylsulfinyl, alkylsulfonyl, alkylamino, or an arylalkyl;
G is CR^{23a}R^{23b}, O, S, or NR^{23c};
L is CR^{24a}R^{24b}, O, S, or NR^{24c};
M is CR^{25a}R^{25b}, C=T, O, S, or NR^{25c};
T is O, S or NR^{25d};
R^{23a}, R^{23b}, R^{23c}, R^{24a}, R^{24b}, R^{24c}, R^{25a}, R^{25b}, R^{25c}, R^{25d} are each independently hydrogen, hydroxyl, alkyl, alkenyl, alkynyl, aryl, arylalkyl, amido, alkylamino, amino, arylamino, alkylcarbonyl, arylcarbonyl, alkylaminocarbonyl, alkoxy, alkoxycarbonyl, alkylcarbonyl, alkyloxycarbonyl, arylcarbonyloxy, aryloxy, phosphate, phosphonato, phosphinato, cyano, acylamino, amidino, imino, sulfhydryl, thiol, alkylthiol, arylthiol, thiocarboxylate, sulfates, alkylsulfinyl, arylsulfinyl, alkylsulfonyl, arylsulfonyl, sulfonato, sulfamoyl, sulfonamido, nitro, trifluoromethyl, cyano, azido, heterocyclyl, alkylsilyl, or arylsilyl;
E is CR^{8d}R^{8e}, S, NR^{8b} or O;
E′ is O, NR^{8f}, or S;
W is CR^{7d}R^{7e}, S, NR^{7b} or O;
W′ is O, NR^{7f}, or S;
Y′ and Y are each independently hydrogen, halogen, hydroxyl, cyano, sulfhydryl, amino, alkyl, alkenyl, alkynyl, alkoxy, alkylthio, alkylsulfinyl, alkylsulfonyl, alkylamino, or an arylalkyl; and pharmaceutically acceptable salts, esters, prodrugs, and enantiomers thereof.

In another embodiment, the invention pertains, at least in part, to tetracycline compounds of formula (IV): 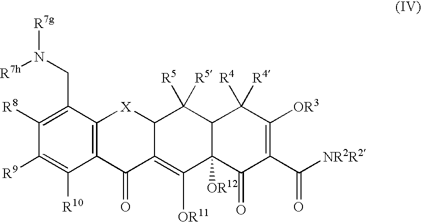
wherein
X is CHC(R¹³Y′Y), C═CR¹³Y, CR^{6′}R⁶, S, NR⁶, or O;
R^{2′}, R^{2″}, R^{4a}, and R^{4b} are each independently hydrogen, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R³, R¹¹ and R¹² are each are each independently hydrogen, alkyl, alkenyl, aryl, alkynyl, aralkyl, acetyl, alkylcarbonyl, alkenylcarbonyl, arylcarbonyl, alkynylcarbonyl, alkyloxycarbonyl, alkenyloxycarbonyl, alkynyloxycarbonyl, aryloxycarbonyl, alkylaminocarbonyl, alkenylaminocarbonyl, alkynylaminocarbonyl, arylaminocarbonyl, alkylthiocarbonyl, alkenylthiocarbonyl, alkynylthiocarbonyl, arylthiocarbonyl, alkyloxythiocarbonyl, alkenyloxythiocarbonyl, alkynyloxythiocarbonyl, aryloxythiocarbonyl, alkylaminothiocarbonyl, alkenylaminothiocarbonyl, alkynylaminothiocarbonyl, arylaminothiocarbonyl, alkylthiothiocarbonyl, alkenylthiothiocarbonyl, alkynylthiothiocarbonyl, or arylthiothiocarbonyl;
R⁴ and R^{4′} are each independently NR^{4a}R^{4b}, alkyl, alkenyl, alkynyl, hydroxyl, halogen, or hydrogen;
R⁵ and R^{5′} are each independently hydroxyl, hydrogen, thiol, alkanoyl, aroyl, alkaroyl, aryl, heteroaromatic, alkyl, alkenyl, alkynyl, alkoxy, alkylthio, alkylsulfinyl, alkylsulfonyl, alkylamino, arylalkyl, alkyl carbonyloxy, or aryl carbonyloxy;
R⁶and R^{6′} are each independently hydrogen, methylene, absent, hydroxyl, halogen, thiol, alkyl, alkenyl, alkynyl, aryl, alkoxy, alkylthio, alkylsulfinyl, alkylsulfonyl, alkylamino, or an arylalkyl;
R^{7g} and R^{7h} are each independently hydrogen, alkyl, alkenyl, aryl, alkynyl, aralkyl, acetyl, alkylcarbonyl, alkenylcarbonyl, arylcarbonyl, alkynylcarbonyl, alkyloxycarbonyl, alkenyloxycarbonyl, alkynyloxycarbonyl, aryloxycarbonyl, alkylaminocarbonyl, alkenylaminocarbonyl, alkynylaminocarbonyl, arylaminocarbonyl, alkylthiocarbonyl, alkenylthiocarbonyl, alkynylthiocarbonyl, arylthiocarbonyl, alkyloxythiocarbonyl, alkenyloxythiocarbonyl, alkynyloxythiocarbonyl, aryloxythiocarbonyl, alkylaminothiocarbonyl, alkenylaminothiocarbonyl, alkynylaminothiocarbonyl, arylaminothiocarbonyl, alkylthiothiocarbonyl, alkenylthiothiocarbonyl, alkynylthiothiocarbonyl, arylthiothiocarbony, or R^{7g} and R^{7h} are linked together to form a ring;
R⁸ is hydrogen, hydroxyl, halogen, thiol, nitro, alkyl, alkenyl, alkynyl, aryl, alkoxy, alkylthio, alkylsulfinyl, alkylsulfonyl, alkylamino, amino, arylalkenyl, arylalkynyl, acyl, aminoalkyl, heterocyclic, thionitroso, or —(CH₂)₀₋₃(NR^{8c})₀₋₁C(=E′)ER^{8a};
E′ is CR^{8d}R^{8e}, S, NR^{8b} or O;
E′ is O, NR^{8f}, or S;
R⁹ is hydrogen, hydroxyl, halogen, thiol, nitro, alkyl, alkenyl, alkynyl, aryl, alkoxy, alkylthio, alkylsulfinyl, alkylsulfonyl, arylalkyl, amido, carboxylate, aminocarbonyl, arylalkenyl, arylalkynyl, acyl, aminoalkyl, heterocyclic, thionitroso, or —(CH₂)₀₋₃NR^{9c}C(=Z′)ZR^{9a};
Z is CR^{9d}R^{9e}, S, NR^{9b} or O;
Z′ is O, S, or NR^{9f};
R^{8a}, R^{8b}, R^{8c}, R^{8d}, R^{8e}, R^{8f}, R^{9a}, R^{9b}, R^{9c}, R^{9d}, R^{9e}, and R^{9f} are each independently hydrogen, acyl, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R¹⁰ is hydrogen;
R¹³ is hydrogen, hydroxy, alkyl, alkenyl, alkynyl, alkoxy, alkylthio, aryl, alkylsulfinyl, alkylsulfonyl, alkylamino, or an arylalkyl;
Y′ and Y are each independently hydrogen, halogen, hydroxyl, cyano, sulfhydryl, amino, alkyl, alkenyl, alkynyl, alkoxy, alkylthio, alkylsulfinyl, alkylsulfonyl, alkylamino, or an arylalkyl;
and pharmaceutically acceptable salts, esters, prodrugs, and enantiomers thereof.

In another embodiment, the invention pertains, at least in part, to tetracycline compounds of formula (V): 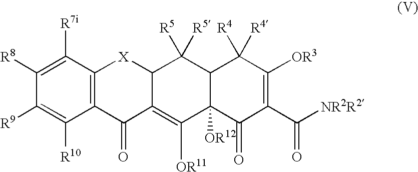
wherein
X is CHC(R¹³Y′Y), C═CR¹³Y, CR^{6′}R⁶, S, NR⁶, or O;
R^{2′}, R^{2″}, R^{4a}, and R^{4b} are each independently hydrogen, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R³, R¹¹ and R¹² are each are each independently hydrogen, alkyl, alkenyl, aryl, alkynyl, aralkyl, acetyl, alkylcarbonyl, alkenylcarbonyl, arylcarbonyl, alkynylcarbonyl, alkyloxycarbonyl, alkenyloxycarbonyl, alkynyloxycarbonyl, aryloxycarbonyl, alkylaminocarbonyl, alkenylaminocarbonyl, alkynylaminocarbonyl, arylaminocarbonyl, alkylthiocarbonyl, alkenylthiocarbonyl, alkynylthiocarbonyl, arylthiocarbonyl, alkyloxythiocarbonyl, alkenyloxythiocarbonyl, alkynyloxythiocarbonyl, aryloxythiocarbonyl, alkylaminothiocarbonyl, alkenylaminothiocarbonyl, alkynylaminothiocarbonyl, arylaminothiocarbonyl, alkylthiothiocarbonyl, alkenylthiothiocarbonyl, alkynylthiothiocarbonyl, or arylthiothiocarbonyl;
R⁴ and R^{4′} are each independently NR^{4a}R^{4b}, alkyl, alkenyl, alkynyl, hydroxyl, halogen, or hydrogen;
R⁵ and R^{5′} are each independently hydroxyl, hydrogen, thiol, alkanoyl, aroyl, alkaroyl, aryl, heteroaromatic, alkyl, alkenyl, alkynyl, alkoxy, alkylthio, alkylsulfinyl, alkylsulfonyl, alkylamino, arylalkyl, alkyl carbonyloxy, or aryl carbonyloxy;
R⁶ and R^{6′} are each independently hydrogen, methylene, absent, hydroxyl, halogen, thiol, alkyl, alkenyl, alkynyl, aryl, alkoxy, alkylthio, alkylsulfinyl, alkylsulfonyl, alkylamino, or an arylalkyl;
R⁷ⁱ is a substituted or unsubstituted heterocycle selected from the group consisting of thiophene, pyrrole, 1,3-oxazole, 1,3-thiazole, 1,3,4-oxadiazole, 1,3,4-thiadiazole, 1,2,3-oxadiazole, 1,2,3-thiadiazole, 1H-1,2,3-triazole, isothiazole, 1,2,4-oxadiazole, 1,2,4-thiadiazole, 1,2,3,4-oxatriazole, 1,2,3,4-thiatriazole, 1H-1,2,3,4-tetraazole, 1,2,3,5-oxatriazole, 1,2,3,5-thiatriazole, furan, imidazol-1-yl, imidazol-4-yl, 1,2,4-triazol-4-yl, 1,2,4-triazol-5-yl, isoxazol-3-yl, isoxazol-5-yl, pyrazol-3-yl, pyrazol-5-yl, thiolane, pyrrolidine, tetrahydrofuran, 4,5-dihydrothiophene, 2-pyrroline, 4,5-dihydrofuran, pyridazine, pyrimidine, pyrazine, 1,2,3-triazine, 1,2,4-triazine, 1,2,4-triazine, 1,3,5-triazine, pyridine, 2H-3,4,5,6-tetrahydropyran, thiane, 1,2-diazaperhydroine, 1,3-diazaperhydroine, piperazine, 1,3-oxazaperhydroine, morpholine, 1,3-thiazaperhydroine, 1,4-thiazaperhydroine, piperidine, 2H-3,4-dihydropyran, 2,3-dihydro-4H-thiin, 1,4,5,6-tetrahydropyridine, 2H-5,6-dihydropyran, 2,3-dihydro-6H-thiin, 1,2,5,6-tetrahydropyridine, 3,4,5,6-tetrahydropyridine, 4H-pyran, 4H-thiin, 1,4-dihydropyridine, 1,4-dithiane, 1,4-dioxane, 1,4-oxathiane, 1,2-oxazolidine, 1,2-thiazolidine, pyrazolidine, 1,3-oxazolidine, 1,3-thiazolidine, imidazolidine, 1,2,4-oxadiazolidine, 1,3,4-oxadiazolidine, 1,2,4-thiadiazolidine, 1,3,4-thiadiazolidine, 1,2,4-triazolidine, 2-imidazoline, 3-imidazoline, 2-pyrazoline, 4-imidazoline, 2,3-dihydroisothiazole, 4,5-dihydroisoxazole, 4,5-dihydroisothiazole, 2,5-dihydroisoxazole, 2,5-dihydroisothiazole, 2,3-dihydroisoxazole, 4,5-dihydrooxazole, 2,3-dihydrooxazole, 2,5-dihydrooxazole, 4,5-dihydrothiazole, 2,3-dihydrothiazole, 2,5-dihydrothiazole, 1,3,4-oxathiazolidine, 1,4,2-oxathiazolidine, 2,3-dihydro-1H-[1,2,3]triazole, 2,5-dihydro-1H-[1,2,3]triazole, 4,5-dihydro-1H-[1,2,3]triazole, 2,3-dihydro-1H-[1,2,4]triazole, 4,5-dihydro-1H-[1,2,4]triazole, 2,3-dihydro-[1,2,4]oxadiazole, 2,5-dihydro-[1,2,4]oxadiazole, 4,5-dihydro-[1,2,4]thiadiazole, 2,3-dihydro-[1,2,4]thidiazole, 2,5-dihydro-[1,2,4]thiadiazole, 4,5-dihydro-[1,2,4]thiadiazole, 2,5-dihydro-[1,2,4]oxadiazole, 2,3-dihydro-[1,2,4]oxadiazole, 4,5-dihydro-[1,2,4]oxadiazole, 2,5-dihydro-[1,2,4]thiadiazole, 2,3-dihydro-[1,2,4]thiadiazole, 4,5-dihydro-[1,2,4]thiadiazole, 2,3-dihydro-[1,3,4]oxadiazole, 2,3-dihydro-[1,3,4]thiadiazole, [1,4,2]oxathiazole, [1,3,4]oxathiazole, 1,3,5-triazaperhydroine, 1,2,4-triazaperhydroine, 1,4,2-dithiazaperhydroine, 1,4,2-dioxazaperhydroine, 1,3,5-oxadiazaperhydroine, 1,2,5-oxadiazaperhydroine, 1,3,4-thiadiazaperhydroine, 1,3,5-thiadiazaperhydroine, 1,2,5-thiadiazaperhydroine, 1,3,4-oxadiazaperhydroine, 1,4,3-oxathiazaperhydroine, 1,4,2-oxathiazaperhydroine, 1,4,5,6-tetrahydropyridazine, 1,2,3,4-tetrahydropyridazine, 1,2,3,6-tetrahydropyridazine, 1,2,5,6-tetrahydropyrimidine, 1,2,3,4-tetrahydropyrimidine, 1,4,5,6-tetrahydropyrimidine, 1,2,3,6-tetrahydropyrazine, 1,2,3,4-tetrahydropyrazine, 5,6-dihydro-4H-[1,2]oxazine, 5,6-dihydro-2H-[1,2]oxazine, 3,6-dihydro-2H-[1,2]oxazine, 3,4-dihydro-2H-[1,2]oxazine, 5,6-dihydro-4H-[1,2]thiazine, 5,6-dihydro-2H-[1,2]thiazine, 3,6-dihydro-2H-[1,2]thiazine, 3,4-dihydro-2H-[1,2]thiazine, 5,6-dihydro-2H-[1,3]oxazine, 5,6-dihydro-4H-[1,3]oxazine, 3,6-dihydro- 2H-[1,3]oxazine, 3,4-dihydro-2H-[1,3]oxazine, 3,6-dihydro-2H-[1,4]oxazine, 3,4-dihydro-2H-[1,4]oxazine, 5,6-dihydro-2H-[1,3]thiazine, 5,6-dihydro-4H-[1,3]thiazine, 3,6-dihydro-2H-[1,3]thiazine, 3,4-dihydro-2H-[1,3]thiazine, 3,6-dihydro-2H-[1,4]thiazine, 3,4-dihydro-2H-[1,4]thiazine, 1,2,3,6-tetrahydro-[1,2,4]triazine, 1,2,3,4-tetrahydro-[1,2,4]triazine, 1,2,3,4-tetrahydro-[1,3,5]triazine, 2,3,4,5-tetrahydro-[1,2,4]triazine, 1,4,5,6-tetrahydro-[1,2,4]triazine, 5,6-dihydro-[1,4,2]dioxazine, 5,6-dihydro-[1,4,2]dioxazine, 5,6-dihydro-[1,4,2]dithiazine, 2,3-dihydro-[1,4,2]dioxazine, 3,4-dihydro-2H-[1,3,4]oxadiazine, 3,6-dihydro-2H-[1,3,4]oxadiazine, 3,4-dihydro-2H-[1,3,5]oxadiazine, 3,6-dihydro-2H-[1,3,5]oxadiazine, 5,6-dihydro-2H-[1,2,5]oxadiazine, 5,6-dihydro-4H-[1,2,5]oxadiazine, 3,4-dihydro-2H-[1,3,4]thiadiazine, 3,6-dihydro-2H-[1,3,4]thiadiazine, 3,4-dihydro-2H-[1,3,5]thiadiazine, 3,6-dihydro-2H-[1,3,5]thiadiazine, 5,6-dihydro-2H-[1,2,5]thiadiazine, 5,6-dihydro-4H-[1,2,5]thiadiazine, 5,6-dihydro-2H-[1,2,3]oxadiazine, 3,6-dihydro-2H-[1,2,5]oxadiazine, 5,6-dihydro-4H-[1,3,4]oxadiazine, 3,4-dihydro-2H-[1,2,5]oxadiazine, 5,6-dihydro-2H-[1,2,3]thiadiazine, 3,6-dihydro-2H-[1,2,5]thiadiazine, 5,6-dihydro-4H-[1,3,4]thiadiazine, 3,4-dihydro-2H-[1,2,5]thiadiazine, 5,6-dihydro-[1,4,3]oxathiazine, 5,6-dihydro-[1,4,2]oxathiazine, 2,3-dihydro-[1,4,3]oxathiazine, 2,3-dihydro-[1,4,2]oxathiazine, 4,5-dihydropyridine, 1,6-dihydropyridine, 5,6-dihydropyridine, 2H-pyran, 2H-thiin, 3,6-dihydropyridine, 2,3-dihydropyridazine, 2,5-dihydropyridazine, 4,5-dihydropyridazine, 1,2-dihydropyridazine, 2,3-dihydropyrimidine, 2,5-dihydropyrimidine, 5,6-dihydropyrimidine, 3,6-dihydropyrimidine, 4,5-dihydropyrazine, 5,6-dihydropyrazine, 3,6-dihydropyrazine, 4,5-dihydropyrazine, 1,4-dihydropyrazine, 1,4-dithiin, 1,4-dioxin, 2H-1,2-oxazine, 6H-1,2-oxazine, 4H-1,2-oxazine, 2H-1,3-oxazine, 4H-1,3-oxazine, 6H- 1,3-oxazine, 2H-1,4-oxazine, 4H-1,4-oxazine, 2H-1,3-thiazine, 2H-1,4-thiazine, 4H-1,2-thiazine, 6H-1,3-thiazine, 4H-1,4-thiazine, 2H-1,2-thiazine, 6H-1,2-thiazine, 1,4-oxathiin, 2H,5H-1,2,3-triazine, 1H,4H-1,2,3-triazine, 4,5-dihydro-1,2,3-triazine, 1H,6H-1,2,3-triazine, 1,2-dihydro-1,2,3-triazine, 2,3-dihydro-1,2,4-triazine, 3H,6H-1,2,4-triazine, 1H,6H-1,2,4-triazine, 3,4-dihydro-1,2,4-triazine, 1H,4H-1,2,4-triazine, 5,6-dihydro-1,2,4-triazine, 4,5 -dihydro-1,2,4-triazine, 2H,5H-1,2,4-triazine, 1,2-dihydro-1,2,4-triazine, 1H,4H-1,3,5-triazine, 1,2-dihydro-1,3,5-triazine, 1,4,2-dithiazine, 1,4,2-dioxazine, 2H-1,3,4-oxadiazine, 2H-1,3,5-oxadiazine, 6H-1,2,5-oxadiazine, 4H-1,3,4-oxadiazine, 4H-1,3,5-oxadiazine, 4H-1,2,5-oxadiazine, 2H-1,3,5-thiadiazine, 6H-1,2,5-thiadiazine, 4H-1,3,4-thiadiazine, 4H-1,3,5-thiadiazine, 4H-1,2,5-thiadiazine, 2H-1,3,4-thiadiazine, 6H-1,3,4-thiadiazine, 6H-1,3,4-oxadiazine and 1,4,2-oxathiazine, wherein the heterocycle is optionally vicinally fused with a saturated or unsaturated 5-, 6- or 7-membered ring containing 0, 1 or 2 atoms independently selected from N, O and S;
R⁸ is hydrogen, hydroxyl, halogen, thiol, nitro, alkyl, alkenyl, alkynyl, aryl, alkoxy, alkylthio, alkylsulfinyl, alkylsulfonyl, alkylamino, amino, arylalkenyl, arylalkynyl, acyl, aminoalkyl, heterocyclic, thionitroso, or —(CH₂)₀₋₃(NR^{8c})₀₋₁C(=E′)ER^{8a};
E is CR^{8d}R^{8e}, S, NR^{8b} or O;
E′ is O, NR^{8f}, or S;
R⁹ is hydrogen, hydroxyl, halogen, thiol, nitro, alkyl, alkenyl, alkynyl, aryl, alkoxy, alkylthio, alkylsulfinyl, alkylsulfonyl, arylalkyl, amido, carboxylate, aminocarbonyl, arylalkenyl, arylalkynyl, acyl, aminoalkyl, heterocyclic, thionitroso, or —(CH₂)₀₋₃(NR^{9c})₀₁C(=Z′)ZR^{9a};
Z is CR^{9d}R^{9e}, S, NR^{9b} or O;
Z′ is O, S, or NR^{9f};
R^{8a}, R^{8b}, R^{8c}, R^{8d}, R^{8e}, R^{9a}, R^{9b}, R^{9c}, R^{9d}, R^{9e}, and R^{9f} are each independently hydrogen, acyl, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R¹⁰ is hydrogen;
R¹³ is hydrogen, hydroxy, alkyl, alkenyl, alkynyl, alkoxy, alkylthio, aryl, alkylsulfinyl, alkylsulfonyl, alkylamino, or an arylalkyl;
Y′ and Y are each independently hydrogen, halogen, hydroxyl, cyano, sulfhydryl, amino, alkyl, alkenyl, alkynyl, alkoxy, alkylthio, alkylsulfinyl, alkylsulfonyl, alkylamino, or an arylalkyl; and pharmaceutically acceptable salts, prodrugs, esters and enantiomers thereof.

The invention also includes, for example, method for treating a tetracycline responsive state in a subject. The methods include administering to a subject an effective amount of a tetracycline compound of the invention (e.g., a compound of any one of formula I, II, III, IV, V, or otherwise described herein).

The invention also pertains, at least in part, to pharmaceutical compositions which comprise an effective amount of a tetracycline compound of the invention (e.g., a tetracycline compound of formula I, II, III, IV, V or otherwise described herein) and a pharmaceutically acceptable carrier.

### DETAILED DESCRIPTION OF THE INVENTION

1. 10-Substituted Tetracycline Compounds

The invention pertains, at least in part, to novel 10-substituted derivatives of tetracyclines.

The term “tetracycline compound” includes many compounds with a similar ring structure to tetracycline. Examples of tetracycline compounds include: tetracycline, oxytetracycline, chlortetracycline, demeclocycline, doxycycline, chelocardin, minocycline, rolitetracycline, lymecycline, sancycline, methacycline, apicycline, clomocycline, guamecycline, meglucycline, mepylcycline, penimepicycline, pipacycline, etamocycline, and penimocycline. Other derivatives and analogues comprising a similar four ring structure are also included. Table 1 depicts tetracycline and several known tetracycline derivatives. The tetracycline compounds may be unsubstituted at any position or further substituted, for example, at the 1, 2, 3, 4, 5, 6, 7, 8, 9, 12 or 13 position of the ring. The C10 position on each of the tetracycline compounds shown in Table 1 is indicated by an arrow.

TABLE I

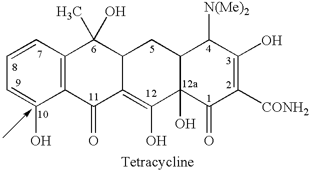

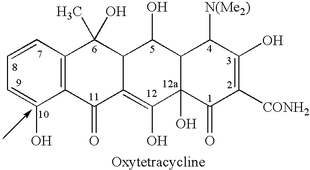

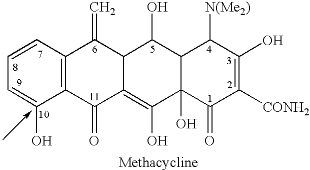

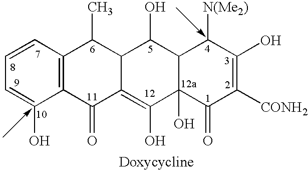

Other tetracycline compounds which may be modified using the methods of the invention include, but are not limited to, 6-demethyl-6-deoxy-4-dedimethylaminotetracycline; tetracyclino-pyrazole; 7-chloro-4-dedimethylaminotetracycline; 4-hydroxy-4-dedimethylaminotetracycline; 12α-deoxy-4-dedimethylaminotetracycline; 5-hydroxy-6α-deoxy-4-dedimethylaminotetracycline; 4-dedimethylamino-12α-deoxyanhydrotetracycline; 7-dimethylamino-6-demethyl-6-deoxy-4-dedimethylaminotetracycline; tetracyclinonitrile; 4-oxo-4-dedimethylaminotetracycline 4,6-hemiketal; 4-oxo-11a C1-4-dedimethylaminotetracycline-4,6-hemiketal; 5a,6-anhydro-4-hydrazon-4-dedimethylamino tetracycline; 4-hydroxyimino-4-dedimethylamino tetracyclines; 4-hydroxyimino-4-dedimethylamino 5a,6-anhydrotetracyclines; 4-amino-4-dedimethylamino-5a, 6 anhydrotetracycline; 4-methylamino-4-dedimethylamino tetracycline; 4-hydrazono-11a-chloro-6-deoxy-6-demethyl-6-methylene-4-dedimethylamino tetracycline; tetracycline quaternary ammonium compounds; anhydrotetracycline betaines; 4-hydroxy-6-methyl pretetramides; 4-keto tetracyclines; 5-keto tetracyclines; 5a, 11a dehydro tetracyclines; 11a C1-6, 12 hemiketal tetracyclines; 11a C1-6-methylene tetracyclines; 6, 13 diol tetracyclines; 6-benzylthiomethylene tetracyclines; 7, 11a-dichloro-6-fluoro-methyl-6-deoxy tetracyclines; 6-fluoro (α)-6-demethyl-6-deoxy tetracyclines; 6-fluoro (β)-6-demethyl-6-deoxy tetracyclines; 6-αacetoxy-6-demethyl tetracyclines; 6-β acetoxy-6-demethyl tetracyclines; 7, 13-epithiotetracyclines; oxytetracyclines; pyrazolotetracyclines; 11a halogens of tetracyclines; 12a formyl and other esters of tetracyclines; 5, 12a esters of tetracyclines; 10, 12a-diesters of tetracyclines; isotetracycline; 12-a-deoxyanhydro tetracyclines; 6-demethyl-12a-deoxy-7-chloroanhydrotetracyclines; B-nortetracyclines; 7-methoxy-6-demethyl-6-deoxytetracyclines; 6-demethyl-6-deoxy-5a-epitetracyclines; 8-hydroxy-6-demethyl-6-deoxy tetracyclines; monardene; chromocycline; 5a methyl-6-demethyl-6-deoxy tetracyclines; 6-oxa tetracyclines, and 6 thia tetracyclines. Other examples of tetracycline compounds which may be used to form dehydrotetracycline compounds of the invention include those described in U.S. Published Application 20040002481, incorporated herein by reference.

The term “10-substituted tetracycline compounds” includes tetracycline compounds which contain a substituent other than a hydroxy at the C10 position. In an embodiment, the 10-substituted tetracycline compound is 10-substituted tetracycline (e.g., wherein R⁴ is NR^{4a}R^{4b}; R^{4a} and R^{4b} are methyl, R^{4′}, R ⁵, and R^{5′} are hydrogen and X is CR⁶R^{6′}, wherein R⁶ is methyl and R^{6′} is hydroxy); 10-substituted doxycycline (e.g., wherein R⁴ is NR^{4a}R^{4b}; R^{4a} and R^{4b} are methyl, R⁵ is hydroxyl, R^{4′} and R^{5′} are hydrogen, and X is CR⁶R^{6′}, wherein R⁶ is methyl and R^{6′} is hydrogen); 10-substituted minocycline (wherein R⁴ is NR^{4a}R^{4b}; R^{4a} and R^{4b} are methyl; R^{4′}, R^{5′}, and R⁵are hydrogen and X is CR⁶R^{6′} wherein R⁶ and R^{6′} are hydrogen atoms, and R⁷ is dimethylamino); or 10-substituted sancycline (wherein R⁴ is NR^{4a}R^{4b}; R^{4a} and R^{4b} are methyl; R^{4′}, R^{5′}, and R⁵ are hydrogen and X is CR⁶R^{6′} wherein R⁶ and R^{6′} are hydrogen atoms. In one embodiment, R⁴ and R^{4′} are each hydrogen or the oxygen of a carbonyl group.

In one embodiment, the 10-substituted tetracycline compounds do not include 4-dimethylamino-3,12,12a-trihydroxy-1,11-dioxo-10-propoxy-1,4,4a,5,5 a,6,11,12a-octahydro-naphthacene-2-carboxylic acid amide; 10-butoxy-4-dimethylamino-3,12,12a-trihydroxy-1,11-dioxo-1,4,4a,5,5a,6,11,12a-octahydro-naphthacene-2-carboxylic acid amide; 10-butoxy-4,7-bis-dimethylamino-3,12,12a-trihydroxy-1,11-dioxo-1,4,4a,5,5a,6,11,12-octahydro-naphthacene-2-carboxylic acid amide; 3,10-bis-benzyloxy-4,7-bis-dimethylamino-12,12a-dihydroxy-1,11-dioxo-1,4,4a,5,5 a,6,11,12a-octahydro-naphthacene-2-carboxylic acid amide; 10-butyloxy-4-dimethylamino-3,12-12a-trihydroxy-1,11-dioxo-7-(4-trifluoromethyl-phenyl)-1,4,4a,5,5a,6,11,12a-octahydro-naphthacene-carboxylic acid; benzenesulfonic acid 9-cyano-7-dimethylamino-8,10a,11-trihydroxy-10,12-dioxo-5,5a,6,6a,7,10,10a,12-octahydro-naphthacen-1-yl ester; 10-butoxy-4-dimethylamino-7-(4-dimethylamino-phenyl)-3,12,12a-trihydroxy-1,11-dioxo-1,4,4a,5,5a,6,11,12a-octahydro-naphthacene-2-carboxylic acid amide; 4-dimethylamino-3,12,12a-trihydroxy-10-(3-hydroxy-propoxy)-1,11-dioxo-1,4,4a,5,5a,6,11,12a-octahydro-naphthacene-2-carboxylic acid amide; 10-butoxy-4-dimethylamino-3,5,12,12a-tetrahydroxy-6-methyl-1,11-dioxo-1,4,4a,5,5a,6,11,12a-octahydro-naphthacene-2-carboxylic acid amide; 3,10-bis-allyloxy-4-dimethylamino-12,12a-dihydroxy-1,11-dioxo-1,4,4a,5,5a,6,11,12a-octahydro-naphthacene-2-carboxylic acid amide; 3,10-bis-benzyloxy-4-dimethylamino-12,12a-dihydroxy-1,11-dioxo-1,4,4a,5,5a,6,11,12a-octahydro-naphthacene-2-carboxylic acid amide; or 10-butoxy-4-dimethylamino-3,12,12a-trihydroxy-7-iodo-1,11-dioxo-1,4,4a,5,5a,6,11,12a-octahydro-naphthacene-2-carboxylic acid amide.

In one embodiment, the invention pertains, in least in part, to tetracycline compounds of formula (I): 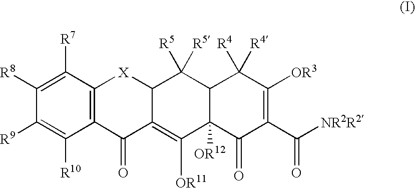
wherein
X is CHC(R¹³Y′Y), C═CR¹³Y, CR^{6′}R⁶, S, NR⁶, or O;
R^{2′}, R^{2″}, R^{4a}, and R^{4b} are each independently hydrogen, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R³, R¹¹ and R¹² are each are each independently hydrogen, alkyl, alkenyl, aryl, alkynyl, aralkyl, acetyl, alkylcarbonyl, alkenylcarbonyl, arylcarbonyl, alkynylcarbonyl, alkyloxycarbonyl, alkenyloxycarbonyl, alkynyloxycarbonyl, aryloxycarbonyl, alkylaminocarbonyl, alkenylaminocarbonyl, alkynylaminocarbonyl, arylaminocarbonyl, alkylthiocarbonyl, alkenylthiocarbonyl, alkynylthiocarbonyl, arylthiocarbonyl, alkyloxythiocarbonyl, alkenyloxythiocarbonyl, alkynyloxythiocarbonyl, aryloxythiocarbonyl, alkylaminothiocarbonyl, alkenylaminothiocarbonyl, alkynylaminothiocarbonyl, arylaminothiocarbonyl, alkylthiothiocarbonyl, alkenylthiothiocarbonyl, alkynylthiothiocarbonyl, or arylthiothiocarbonyl;
R⁴ and R^{4′} are each independently NR^{4a}R^{4b}, alkyl, alkenyl, alkynyl, hydroxyl, halogen, or hydrogen;
R⁵ and R^{5′} are each independently hydroxyl, hydrogen, thiol, alkanoyl, aroyl, alkaroyl, aryl, heteroaromatic, alkyl, alkenyl, alkynyl, alkoxy, alkylthio, alkylsulfinyl, alkylsulfonyl, alkylamino, arylalkyl, alkyl carbonyloxy, or aryl carbonyloxy;
R⁶and R^{6′} are each independently hydrogen, methylene, absent, hydroxyl, halogen, thiol, alkyl, alkenyl, alkynyl, aryl, alkoxy, alkylthio, alkylsulfinyl, alkylsulfonyl, alkylamino, or an arylalkyl;
R⁷ is hydrogen, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino, acyl, acylamino, amidino, imino, sulfhydryl, alkylthio, arylthio, thiocarboxylate, sulfates, alkylsulfinyl, arylsulfinyl, alkylsulfonyl, arylsulfonyl, sulfonato, sulfamoyl, sulfonamido, nitro, trifluoromethyl, cyano, azido, alkylaryl, aryl, a heterocyclic moiety or —(CH₂)₀₋₃(NR^{7c})₀₋₁C(′W′)WR^{7a};
R⁸ is hydrogen, hydroxyl, halogen, thiol, nitro, alkyl, alkenyl, alkynyl, aryl, alkoxy, alkylthio, alkylsulfinyl, alkylsulfonyl, alkylamino, amino, arylalkenyl, arylalkynyl, acyl, aminoalkyl, heterocyclic, thionitroso, or —(CH₂)₀₋₃(NR^{8c})₀₋₁C(=E′)ER^{8a};
R⁹ is hydrogen, hydroxyl, halogen, thiol, nitro, alkyl, alkenyl, alkynyl, aryl, alkoxy, alkylthio, alkylsulfinyl, alkylsulfonyl, arylalkyl, amido, carboxylate, aminocarbonyl, arylalkenyl, arylalkynyl, acyl, aminoalkyl, heterocyclic, thionitroso, or —(CH₂)₀₋₃(NR^{9c})₀₋₁C(=Z′)ZR^{9a};
R¹⁰ is hydrogen, alkyl, alkenyl, alkynyl, halogen, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, arylaminocarbonyl, alkylthiocarbonyl, arylthiocarbonyl; phosphate, phosphonato, phosphinato, cyano, amino, acylamino, amidino, imino, sulthydryl, alkylthio, arylthio, thiocarboxylate, sulfates, alkylsulfinyl, arylsulfinyl, alkylsulfonyl, arylsulfonyl, sulfonato, sulfamoyl, sulfonamido, nitro, trifluoromethyl, cyano, azido, alkylaryl, aryl or a heterocyclic moiety;
R^{7a}, R^{7b}, R^{7c}, R^{7d}, R^{7e}, R^{7f}, R^{8a}, R^{8b}, R^{8c}, R^{8d}, R^{8e}, R^{8f}, R^{9a}, R^{9b}, R^{9c}, R^{9d}, R^{9e}, and R^{9f} are each independently hydrogen, acyl, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R¹³ is hydrogen, hydroxy, alkyl, alkenyl, alkynyl, alkoxy, alkylthio, aryl, alkylsulfinyl, alkylsulfonyl, alkylamino, or an arylalkyl;
E is CR^{8d}R^{8e}, S, NR^{8b} or O;
E′ is O, NR^{8f} or S;
W is CR^{7d}R^{7e}, S, NR^{7b} or O;
W′ is O, NR^{7f} or S;
Y′ and Y are each independently hydrogen, halogen, hydroxyl, cyano, sulfhydryl, amino, alkyl, alkenyl, alkynyl, alkoxy, alkylthio, alkylsulfinyl, alkylsulfonyl, alkylamino, or an arylalkyl;
Z is CR^{9d}R^{9e}, S, NR^{9b} or O;
Z′ is O, S, or NR^{9f} and pharmaceutically acceptable salts, esters and enantiomers thereof.

In one embodiment, the compound of formula (I) is not 10-deoxysancycline.

In another embodiment, the invention pertains, in least in part, to tetracycline compounds of formula (II): 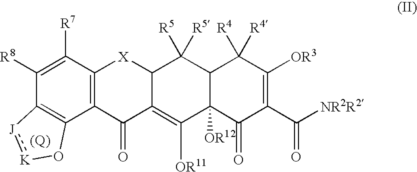
wherein
X is CHC(R¹³Y′Y), C═CR¹³Y, CR^{6′}R⁶, S, NR⁶, or O;
R^{2′}, R^{2″}, R^{4a}, and R^{4b} are each independently hydrogen, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R³, R¹¹ and R¹² are each are each independently hydrogen, alkyl, alkenyl, aryl, alkynyl, aralkyl, acetyl, alkylcarbonyl, alkenylcarbonyl, arylcarbonyl, alkynylcarbonyl, alkyloxycarbonyl, alkenyloxycarbonyl, alkynyloxycarbonyl, aryloxycarbonyl, alkylaminocarbonyl, alkenylaminocarbonyl, alkynylaminocarbonyl, arylaminocarbonyl, alkylthiocarbonyl, alkenylthiocarbonyl, alkynylthiocarbonyl, arylthiocarbonyl, alkyloxythiocarbonyl, alkenyloxythiocarbonyl, alkynyloxythiocarbonyl, aryloxythiocarbonyl, alkylaminothiocarbonyl, alkenylaminothiocarbonyl, alkynylaminothiocarbonyl, arylaminothiocarbonyl, alkylthiothiocarbonyl, alkenylthiothiocarbonyl, alkynylthiothiocarbonyl, or arylthiothiocarbonyl;
R⁴ and R^{4′} are each independently NR^{4a}R^{4b}, alkyl, alkenyl, alkynyl, hydroxyl, halogen, or hydrogen;
R⁵ and R^{5′} are each independently hydroxyl, hydrogen, thiol, alkanoyl, aroyl, alkaroyl, aryl, heteroaromatic, alkyl, alkenyl, alkynyl, alkoxy, alkylthio, alkylsulfinyl, alkylsulfonyl, alkylamino, arylalkyl, alkyl carbonyloxy, or aryl carbonyloxy;
R⁶ and R^{6′} are each independently hydrogen, methylene, absent, hydroxyl, halogen, thiol, alkyl, alkenyl, alkynyl, aryl, alkoxy, alkylthio, alkylsulfinyl, alkylsulfonyl, alkylamino, or an arylalkyl;
R⁷ is hydrogen, alkyl, alkenyl, alkynyl, halogen, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, arylaminocarbonyl, alkylthiocarbonyl, arylthiocarbonyl; phosphate, phosphonato, phosphinato, cyano, amino, acylamino, amidino, imino, sulfhydryl, alkylthio, arylthio, thiocarboxylate, sulfates, alkylsulfinyl, arylsulfinyl, alkylsulfonyl, arylsulfonyl, sulfonato, sulfamoyl, sulfonamido, nitro, trifluoromethyl, cyano, azido, alkylaryl, aryl, a heterocyclic moiety, or —(CH₂)₀₋₃(NR^{7c})₀₋₁C(═W′)WR^{7a};
R⁸ is hydrogen, hydroxyl, halogen, thiol, nitro, alkyl, alkenyl, alkynyl, aryl, alkoxy, alkylthio, alkylsulfinyl, alkylsulfonyl, alkylamino, amino, arylalkenyl, arylalkynyl, acyl, aminoalkyl, heterocyclic, thionitroso, or —(CH₂)₀₋₃(NR^{8c})₀₋₁C(=E′)ER^{8a};
R^{7a}, R^{7b}, R^{7c}, R^{7d}, R^{7e}, R^{7f}, R^{8a}, R^{8b}, R^{8c}, R^{8d}, R^{8e}, R^{8f} are each independently hydrogen, acyl, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R¹³ is hydrogen, hydroxy, alkyl, alkenyl, alkynyl, alkoxy, alkylthio, aryl, alkylsulfinyl, alkylsulfonyl, alkylamino, or an arylalkyl;
J is CR^{21a}R^{21b}, O, S, or NR^{21c};
K is CR^{22a}R^{22b}, O, S, or NR^{22c}
R^{21a}, R^{21b}, R^{21c}, R^{22a}, R^{22b}, R^{22c} are each independently hydrogen, hydroxyl, alkyl, alkenyl, alkynyl, aryl, arylalkyl, amido, alkylamino, amino, arylamino, alkylcarbonyl, arylcarbonyl, alkylaminocarbonyl, alkoxy, alkoxycarbonyl, alkylcarbonyl, alkyloxycarbonyl, arylcarbonyloxy, aryloxy, phosphate, phosphonato, phosphinato, cyano, acylamino, amidino, imino, sulfhydryl, thiol, alkylthiol, arylthiol, thiocarboxylate, sulfates, alkylsulfinyl, arylsulfinyl, alkylsulfonyl, arylsulfonyl, sulfonato, sulfamoyl, sulfonamido, nitro, trifluoromethyl, cyano, azido, heterocyclyl, alkylsilyl, arylsilyl, or absent;
Q is a double bond when J is CR^{21a}R^{21b}, K is CR^{22a}R^{22b} and R^{21b} and R^{22b} are absent;
Q is a double bond when J is NR^{21c}, K is CR^{22a}R^{22b} and R^{21c} and R^{22b} are absent;
Q is a double bond when J is CR^{21a}R^{21b}, K is NR^{22c}, and R^{21b} and R^{22c} are absent;
Q is a single bond when J is CR^{21a}R^{21b}, O, S, or NR^{21c}, K is CR^{22a}R^{22b}, O, S, or NR^{22c} and R^{21a}, R^{21b}, R^{21c}, R^{22a}, R^{22b}, R^{22c} are each independently hydrogen, hydroxyl, alkyl, alkenyl, alkynyl, aryl, arylalkyl, amido, alkylamino, amino, arylamino, alkylcarbonyl, arylcarbonyl, alkylaminocarbonyl, alkoxy, alkoxycarbonyl, alkylcarbonyl, alkyloxycarbonyl, arylcarbonyloxy, aryloxy, phosphate, phosphonato, phosphinato, cyano, acylamino, amidino, imino, sulfhlydryl, thiol, alkylthiol, arylthiol, thiocarboxylate, sulfates, alkylsulfinyl, arylsulfinyl, alkylsulfonyl, arylsulfonyl, sulfonato, sulfamoyl, sulfonamido, nitro, trifluoromethyl, cyano, azido, heterocyclyl, alkylsilyl, or arylsilyl;
E is CR^{8d}R^{8e}, S, NR^{8b} or O;
E′ is O, NR^{8f}, or S;
W is CR^{7d}R^{7e}, S, NR^{7b} or O;
W′ is O, NR^{7f}, or S;
Y′ and Y are each independently hydrogen, halogen, hydroxyl, cyano, sulfhydryl, amino, alkyl, alkenyl, alkynyl, alkoxy, alkylthio, alkylsulfinyl, alkylsulfonyl, alkylamino, or an arylalkyl; and pharmaceutically acceptable salts, esters and enantiomers thereof.

In yet another embodiment, the invention pertains, at least in part, to tetracycline compounds of formula (III): 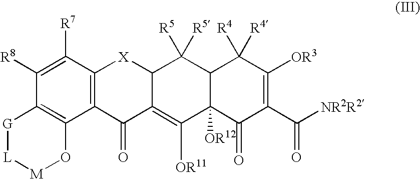
wherein
X is CHC(R¹³Y′Y), C═CR¹³Y, CR^{6′}R⁶, S, NR⁶, or O;
R^{2′}, R^{2″}, R^{4a}, and R^{4b} are each independently hydrogen, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R³, R¹¹ and R¹² are each are each independently hydrogen, alkyl, alkenyl, aryl, alkynyl, aralkyl, acetyl, alkylcarbonyl, alkenylcarbonyl, arylcarbonyl, alkynylcarbonyl, alkyloxycarbonyl, alkenyloxycarbonyl, alkynyloxycarbonyl, aryloxycarbonyl, alkylaminocarbonyl, alkenylaminocarbonyl, alkynylaminocarbonyl, arylaminocarbonyl, alkylthiocarbonyl, alkenylthiocarbonyl, alkynylthiocarbonyl, arylthiocarbonyl, alkyloxythiocarbonyl, alkenyloxythiocarbonyl, alkynyloxythiocarbonyl, aryloxythiocarbonyl, alkylaminothiocarbonyl, alkenylaminothiocarbonyl, alkynylaminothiocarbonyl, arylaminothiocarbonyl, alkylthiothiocarbonyl, alkenylthiothiocarbonyl, alkynylthiothiocarbonyl, or arylthiothiocarbonyl;
R⁴ and R^{4′} are each independently NR^{4a}R^{4b}, alkyl, alkenyl, alkynyl, hydroxyl, halogen, or hydrogen;
R⁵ and R^{5′} are each independently hydroxyl, hydrogen, thiol, alkanoyl, aroyl, alkaroyl, aryl, heteroaromatic, alkyl, alkenyl, alkynyl, alkoxy, alkylthio, alkylsulfinyl, alkylsulfonyl, alkylamino, arylalkyl, alkyl carbonyloxy, or aryl carbonyloxy;
R⁶ and R^{6′} are each independently hydrogen, methylene, absent, hydroxyl, halogen, thiol, alkyl, alkenyl, alkynyl, aryl, alkoxy, alkylthio, alkylsulfinyl, alkylsulfonyl, alkylamino, or an arylalkyl;
R⁷ is hydrogen, hydroxyl, halogen, thiol, nitro, alkyl, alkenyl, alkynyl, aryl, alkoxy, alkylthio, alkylsulfinyl, alkylsulfonyl, arylalkyl, amino, arylalkenyl, arylalkynyl, acyl, aminoalkyl, heterocyclic, thionitroso, or —(CH₂)₀₋₃(NR^{7c})₀₋₁C(═W′)WR^{7a};
R⁸ is hydrogen, hydroxyl, halogen, thiol, nitro, alkyl, alkenyl, alkynyl, aryl, alkoxy, alkylthio, alkylsulfinyl, alkylsulfonyl, alkylamino, amino, arylalkenyl, arylalkynyl, acyl, aminoalkyl, heterocyclic, thionitroso, or —(CH₂)₀₋₃(NR^{8c})₀₋₁C(=E′)ER^{8a};
R^{7a}, R^{7b}, R^{7c}, R^{7d}, R^{7e}, R^{7f}, R^{8a}, R^{8b}, R^{8c}, R^{8d}, R^{8f} are each independently hydrogen, acyl, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R¹³ is hydrogen, hydroxy, alkyl, alkenyl, alkynyl, alkoxy, alkylthio, aryl, alkylsulfinyl, alkylsulfonyl, alkylamino, or an arylalkyl;
G is CR^{23a}R^{23b}, O, S, or NR^{23c};
L is CR^{24a}R^{24b}, O, S, or NR^{24c};
M is CR^{25a}R^{25b}, C=T, O, S, or NR^{25c};
T is O, S or NR^{25d};
R^{23a}, R^{23b}, R^{23c}, R^{24a}, R^{24b}, R^{24c}, R^{25a}, R^{25b}, R^{25c}, R^{25d} are each independently hydrogen, hydroxyl, alkyl, alkenyl, alkynyl, aryl, arylalkyl, amido, alkylamino, amino, arylamino, alkylcarbonyl, arylcarbonyl, alkylaminocarbonyl, alkoxy, alkoxycarbonyl, alkylcarbonyl, alkyloxycarbonyl, arylcarbonyloxy, aryloxy, phosphate, phosphonato, phosphinato, cyano, acylamino, amidino, imino, sulfhydryl, thiol, alkylthiol, arylthiol, , thiocarboxylate, sulfates, alkylsulfinyl, arylsulfinyl, alkylsulfonyl, arylsulfonyl, sulfonato, sulfamoyl, sulfonamido, nitro, trifluoromethyl, cyano, azido, heterocyclyl, alkylsilyl, or arylsilyl;
E is CR^{8d}R^{8e}, S, NR^{8b} or O;
E′ is O, NR^{8f}, or S;
W is CR^{7d}R^{7e}, S, NR^{7b} or O;
W′ is O, NR^{7f}, or S;
Y′ and Y are each independently hydrogen, halogen, hydroxyl, cyano, sulfhydryl, amino, alkyl, alkenyl, alkynyl, alkoxy, alkylthio, alkylsulfinyl, alkylsulfonyl, alkylamino, or an arylalkyl; and pharmaceutically acceptable salts, esters and enantiomers thereof.

In another embodiment, the invention pertains, at least in part, to tetracycline compounds of formula (IV): 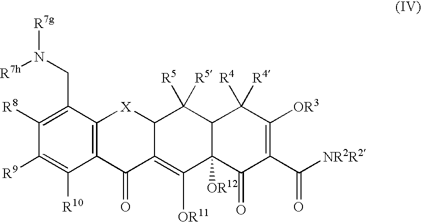
wherein
X is CHC(R¹³Y′Y), C═CR¹³Y, CR^{6′}R⁶, S, NR⁶, or O;
R^{2′}, R^{2″}, R^{4a}, and R^{4b} are each independently hydrogen, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R³, R¹¹ and R¹² are each are each independently hydrogen, alkyl, alkenyl, aryl, alkynyl, aralkyl, acetyl, alkylcarbonyl, alkenylcarbonyl, arylcarbonyl, alkynylcarbonyl, alkyloxycarbonyl, alkenyloxycarbonyl, alkynyloxycarbonyl, aryloxycarbonyl, alkylaminocarbonyl, alkenylaminocarbonyl, alkynylaminocarbonyl, arylaminocarbonyl, alkylthiocarbonyl, alkenylthiocarbonyl, alkynylthiocarbonyl, arylthiocarbonyl, alkyloxythiocarbonyl, alkenyloxythiocarbonyl, alkynyloxythiocarbonyl, aryloxythiocarbonyl, alkylaminothiocarbonyl, alkenylaminothiocarbonyl, alkynylaminothiocarbonyl, arylaminothiocarbonyl, alkylthiothiocarbonyl, alkenylthiothiocarbonyl, alkynylthiothiocarbonyl, or arylthiothiocarbonyl;
R⁴ and R^{4′} are each independently NR^{4a}R^{4b}, alkyl, alkenyl, alkynyl, hydroxyl, halogen, or hydrogen;
R⁵ and R^{5′} are each independently hydroxyl, hydrogen, thiol, alkanoyl, aroyl, alkaroyl, aryl, heteroaromatic, alkyl, alkenyl, alkynyl, alkoxy, alkylthio, alkylsulfinyl, alkylsulfonyl, alkylamino, arylalkyl, alkyl carbonyloxy, or aryl carbonyloxy;
R⁶ and R^{6′} are each independently hydrogen, methylene, absent, hydroxyl, halogen, thiol, alkyl, alkenyl, alkynyl, aryl, alkoxy, alkylthio, alkylsulfinyl, alkylsulfonyl, alkylamino, or an arylalkyl;
R^{7g} and R^{7h} are each independently hydrogen, alkyl, alkenyl, aryl, alkynyl, aralkyl, acetyl, alkylcarbonyl, alkenylcarbonyl, arylcarbonyl, alkynylcarbonyl, alkyloxycarbonyl, alkenyloxycarbonyl, alkynyloxycarbonyl, aryloxycarbonyl, alkylaminocarbonyl, alkenylaminocarbonyl, alkynylaminocarbonyl, arylaminocarbonyl, alkylthiocarbonyl, alkenylthiocarbonyl, alkynylthiocarbonyl, arylthiocarbonyl, alkyloxythiocarbonyl, alkenyloxythiocarbonyl, alkynyloxythiocarbonyl, aryloxythiocarbonyl, alkylaminothiocarbonyl, alkenylaminothiocarbonyl, alkynylaminothiocarbonyl, arylaminothiocarbonyl, alkylthiothiocarbonyl, alkenylthiothiocarbonyl, alkynylthiothiocarbonyl, arylthiothiocarbony, or R^{7g} and R^{7h} are linked together to form a ring;
R⁸ is hydrogen, hydroxyl, halogen, thiol, nitro, alkyl, alkenyl, alkynyl, aryl, alkoxy, alkylthio, alkylsulfinyl, alkylsulfonyl, alkylamino, amino, arylalkenyl, arylalkynyl, acyl, aminoalkyl, heterocyclic, thionitroso, or —(CH₂)₀₋₃(NR^{8c})₀₋₁C(=E′)ER^{8a};
E is CR^{8d}R^{8e}, S, NR^{8b} or O;
E′ is O, NR^{8f}, or S;
R⁹ is hydrogen, hydroxyl, halogen, thiol, nitro, alkyl, alkenyl, alkynyl, aryl, alkoxy, alkylthio, alkylsulfinyl, alkylsulfonyl, arylalkyl, amido, carboxylate, aminocarbonyl, arylalkenyl, arylalkynyl, acyl, aminoalkyl, heterocyclic, thionitroso, or —(CH₂)₀₋₃(NR^{9c})₀₋₁C(=Z′)ZR^{9a};
Z is CR^{9d}R^{9e}, S, NR^{9b} or O;
Z′ is O, S, or NR^{9f};
R^{8a}, R^{8b}, R^{8c}, R^{8d}, R^{8e}, R^{8f}, R^{9a}, R^{9b}, R^{9c}, R^{9d}, R^{9e}, and R^{9f} are each independently hydrogen, acyl, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R¹⁰ is hydrogen;
R¹³ is hydrogen, hydroxy, alkyl, alkenyl, alkynyl, alkoxy, alkylthio, aryl, alkylsulfinyl, alkylsulfonyl, alkylamino, or an arylalkyl;
Y′ and Y are each independently hydrogen, halogen, hydroxyl, cyano, sulfhydryl, amino, alkyl, alkenyl, alkynyl, alkoxy, alkylthio, alkylsulfinyl, alkylsulfonyl, alkylamino, or an arylalkyl; and pharmaceutically acceptable salts, esters and enantiomers thereof.

In another embodiment, the invention pertains, at least in part, to tetracycline compounds of formula (V): 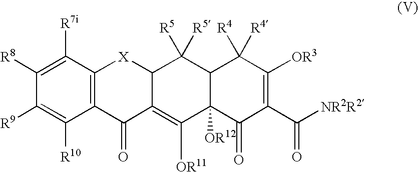
wherein
X is CHC(R¹³Y′Y), C═CR¹³Y, CR^{6′}R⁶, S, NR⁶, or O;
R^{2′}, R^{2″}, R^{4a}, and R^{4b} are each independently hydrogen, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R³, R¹¹ and R¹² are each are each independently hydrogen, alkyl, alkenyl, aryl, alkynyl, aralkyl, acetyl, alkylcarbonyl, alkenylcarbonyl, arylcarbonyl, alkynylcarbonyl, alkyloxycarbonyl, alkenyloxycarbonyl, alkynyloxycarbonyl, aryloxycarbonyl, alkylaminocarbonyl, alkenylaminocarbonyl, alkynylaminocarbonyl, arylaminocarbonyl, alkylthiocarbonyl, alkenylthiocarbonyl, alkynylthiocarbonyl, arylthiocarbonyl, alkyloxythiocarbonyl, alkenyloxythiocarbonyl, alkynyloxythiocarbonyl, aryloxythiocarbonyl, alkylaminothiocarbonyl, alkenylaminothiocarbonyl, alkynylaminothiocarbonyl, arylaminothiocarbonyl, alkylthiothiocarbonyl, alkenylthiothiocarbonyl, alkynylthiothiocarbonyl, or arylthiothiocarbonyl;
R⁴ and R^{4′} are each independently NR^{4a}R^{4b}, alkyl, alkenyl, alkynyl, hydroxyl, halogen, or hydrogen;
R⁵ and R^{5′} are each independently hydroxyl, hydrogen, thiol, alkanoyl, aroyl, alkaroyl, aryl, heteroaromatic, alkyl, alkenyl, alkynyl, alkoxy, alkylthio, alkylsulfinyl, alkylsulfonyl, alkylamino, arylalkyl, alkyl carbonyloxy, or aryl carbonyloxy;
R⁶ and R^{6′} are each independently hydrogen, methylene, absent, hydroxyl, halogen, thiol, alkyl, alkenyl, alkynyl, aryl, alkoxy, alkylthio, alkylsulfinyl, alkylsulfonyl, alkylamino, or an arylalkyl;
R⁷ⁱ is a substituted or unsubstituted heterocycle selected from the group consisting of thiophene, pyrrole, 1,3-oxazole, 1,3-thiazole, 1,3,4-oxadiazole, 1,3,4-thiadiazole, 1,2,3-oxadiazole, 1,2,3-thiadiazole, 1H-1,2,3-triazole, isothiazole, 1,2,4-oxadiazole, 1,2,4-thiadiazole, 1,2,3,4-oxatriazole, 1,2,3,4-thiatriazole, 1H-1,2,3,4-tetraazole, 1,2,3,5-oxatriazole, 1,2,3,5-thiatriazole, furan, imidazol-1-yl, imidazol-4-yl, 1,2,4-triazol-4-yl, 1,2,4-triazol-5-yl, isoxazol-3-yl, isoxazol-5-yl, pyrazol-3-yl, pyrazol-5-yl, thiolane, pyrrolidine, tetrahydrofuiran, 4,5-dihydrothiophene, 2-pyrroline, 4,5-dihydrofuran, pyridazine, pyrimidine, pyrazine, 1,2,3-triazine, 1,2,4-triazine, 1,2,4-triazine, 1,3,5-triazine, pyridine, 2H-3,4,5,6-tetrahydropyran, thiane, 1,2-diazaperhydroine, 1,3-diazaperhydroine, piperazine, 1,3-oxazaperhydroine, morpholine, 1,3-thiazaperhydroine, 1,4-thiazaperhydroine, piperidine, 2H-3,4-dihydropyran, 2,3-dihydro-4H-thiin, 1,4,5,6-tetrahydropyridine, 2H-5,6-dihydropyran, 2,3-dihydro-6H-thiin, 1,2,5,6-tetrahydropyridine, 3,4,5,6-tetrahydropyridine, 4H-pyran, 4H-thiin, 1,4-dihydropyridine, 1,4-dithiane, 1,4-dioxane, 1,4-oxathiane, 1,2-oxazolidine, 1,2-thiazolidine, pyrazolidine, 1,3-oxazolidine, 1,3-thiazolidine, imidazolidine, 1,2,4-oxadiazolidine, 1,3,4-oxadiazolidine, 1,2,4-thiadiazolidine, 1,3,4-thiadiazolidine, 1,2,4-triazolidine, 2-imidazoline, 3-imidazoline, 2-pyrazoline, 4-imidazoline, 2,3-dihydroisothiazole, 4,5-dihydroisoxazole, 4,5-dihydroisothiazole, 2,5-dihydroisoxazole, 2,5-dihydroisothiazole, 2,3-dihydroisoxazole, 4,5-dihydrooxazole, 2,3-dihydrooxazole, 2,5-dihydrooxazole, 4,5-dihydrothiazole, 2,3-dihydrothiazole, 2,5-dihydrothiazole, 1,3,4-oxathiazolidine, 1,4,2-oxathiazolidine, 2,3-dihydro-1H-[1,2,3]triazole, 2,5-dihydro-1H-[1,2,3]triazole, 4,5-dihydro-1H-[1,2,3]triazole, 2,3-dihydro-1H-[1,2,4]triazole, 4,5-dihydro-1H-[1,2,4]triazole, 2,3-dihydro-[1,2,4]oxadiazole, 2,5-dihydro-[1,2,4]oxadiazole, 4,5-dihydro-[1,2,4]thiadiazole, 2,3-dihydro-[1,2,4]thidiazole, 2,5-dihydro-[1,2,4]thiadiazole, 4,5-dihydro-[1,2,4]thiadiazole, 2,5-dihydro-[1,2,4]oxadiazole, 2,3-dihydro-[1,2,4]oxadiazole, 4,5-dihydro-[1,2,4]oxadiazole, 2,5-dihydro-[1,2,4]thiadiazole, 2,3-dihydro-[1,2,4]thiadiazole, 4,5-dihydro-[1,2,4]thiadiazole, 2,3-dihydro-[1,3,4]oxadiazole, 2,3-dihydro-[1,3,4]thiadiazole, [1,4,2]oxathiazole, [1,3,4]oxathiazole, 1,3,5-triazaperhydroine, 1,2,4-triazaperhydroine, 1,4,2-dithiazaperhydroine, 1,4,2-dioxazaperhydroine, 1,3,5-oxadiazaperhydroine, 1,2,5-oxadiazaperhydroine, 1,3,4-thiadiazaperhydroine, 1,3,5-thiadiazaperhydroine, 1,2,5-thiadiazaperhydroine, 1,3,4-oxadiazaperhydroine, 1,4,3-oxathiazaperhydroine, 1,4,2-oxathiazaperhydroine, 1,4,5,6-tetrahydropyridazine, 1,2,3,4-tetrahydropyridazine, 1,2,3,6-tetrahydropyridazine, 1,2,5,6-tetrahydropyrimidine, 1,2,3,4-tetrahydropyrimidine, 1,4,5,6-tetrahydropyrimidine, 1,2,3,6-tetrahydropyrazine, 1,2,3,4-tetrahydropyrazine, 5,6-dihydro-4H-[1,2]oxazine, 5,6-dihydro-2H-[1,2]oxazine, 3,6-dihydro-2H-[1,2]oxazine, 3,4-dihydro-2H-[1,2]oxazine, 5,6-dihydro-4H-[1,2]thiazine, 5,6-dihydro-2H-[1,2]thiazine, 3,6-dihydro-2H-[1,2]thiazine, 3,4-dihydro-2H-[1,2]thiazine, 5,6-dihydro-2H-[1,3]oxazine, 5,6-dihydro-4H-[1,3]oxazine, 3,6-dihydrdo-2H-[1,3]oxazine, 3,4-dihydro-2H-[1,3]oxazine, 3,6-dihydro-2H-[1,4]oxazine, 3,4-dihydro-2H-[1,4]oxazine, 5,6-dihydro-2H-[1,3]thiazine, 5,6-dihydro-4H-[1,3]thiazine, 3,6-dihydro-2H-[1,3]thiazine, 3,4-dihydro-2H-[1,3]thiazine, 3,6-dihydro-2H-[1,4]thiazine, 3,4-dihydro-2H-[1,4]thiazine, 1,2,3,6-tetrahydro-[1,2,4]triazine, 1,2,3,4-tetrahydro-[1,2,4]triazine, 1,2,3,4-tetrahydro-[1,3,5]triazine, 2,3,4,5-tetrahydro-[1,2,4]triazine, 1,4,5,6-tetrahydro-[1,2,4]triazine, 5,6-dihydro-[1,4,2]dioxazine, 5,6-dihydro-[1,4,2]dioxazine, 5,6-dihydro-[1,4,2]dithiazine, 2,3-dihydro-[1,4,2]dioxazine, 3,4-dihydro-2H-[1,3,4]oxadiazine, 3,6-dihydro-2H-[1,3,4]oxadiazine, 3,4-dihydro-2H-[1,3,5]oxadiazine, 3,6-dihydro-2H-[1,3,5]oxadiazine, 5,6-dihydro-2H-[1,2,5]oxadiazine, 5,6-dihydro-4H-[1,2,5]oxadiazine, 3,4-dihydro-2H-[1,3,4]thiadiazine, 3,6-dihydro-2H-[1,3,4]thiadiazine, 3,4-dihydro-2H-[1,3,5]thiadiazine, 3,6-dihydro-2H-[1,3,5]thiadiazin, 5,6-dihydro-2H-[1,2,5]thiadiazine, 5,6-dihydro-4H-[1,2,5]thiadiazine, 5,6-dihydro-2H-[1,2,3]oxadiazine, 3,6-dihydro-2H-[1,2,5]oxadiazine, 5,6-dihydro-4H-[1,3,4]oxadiazine, 3,4-dihydro-2H-[1,2,5]oxadiazine, 5,6-dihydro-2H-[1,2,3]thiadiazine, 3,6-dihydro-2H-[1,2,5]thiadiazine, 5,6-dihydro-4H-[1,3,4]thiadiazine, 3,4-dihydro-2H-[1,2,5]thiadiazine, 5,6-dihydro-[1,4,3]oxathiazine, 5,6-dihydro-[1,4,2]oxathiazine, 2,3-dihydro-[1,4,3]oxathiazine, 2,3-dihydro-[1,4,2]oxathiazine, 4,5-dihydropyridine, 1,6-dihydropyridine, 5,6-dihydropyridine, 2H-pyran, 2H-thiin, 3,6-dihydropyridine, 2,3-dihydropyridazine, 2,5-dihydropyridazine, 4,5-dihydropyridazine, 1,2-dihydropyridazine, 2,3-dihydropyrimidine, 2,5-dihydropyrimidine, 5,6-dihydropyrimidine, 3,6-dihydropyrimidine, 4,5-dihydropyrazine, 5,6-dihydropyrazine, 3,6-dihydropyrazine, 4,5-dihydropyrazine, 1,4-dihydropyrazine, 1,4-dithiin, 1,4-dioxin, 2H-1,2-oxazine, 6H-1,2-oxazine, 4H-1,2-oxazine, 2H-1,3-oxazine, 4H-1,3-oxazine, 6H-1,3-oxazine, 2H-1,4-oxazine, 4H-1,4-oxazine, 2H-1,3-thiazine, 2H-1,4-thiazine, 4H-1,2-thiazine, 6H-1,3-thiazine, 4H-1,4-thiazine, 2H-1,2-thiazine, 6H-1,2-thiazine, 1,4-oxathiin, 2H,5H-1,2,3-triazine, 1H,4H-1,2,3-triazine, 4,5-dihydro-1,2,3-triazine, 1H,6H-1,2,3-triazine, 1,2-dihydro-1,2,3-triazine, 2,3-dihydro-1,2,4-triazine, 3H,6H-1,2,4-triazine, 1H,6H-1,2,4-triazine, 3,4-dihydro-1,2,4-triazine, 1H,4H-1,2,4-triazine, 5,6-dihydro-1,2,4-triazine, 4,5-dihydro-1,2,4-triazine, 2H,5H-1,2,4-triazine, 1,2-dihydro-1,2,4-triazine, 1H,4H-1,3,5-triazine, 1,2-dihydro-1,3,5-triazine, 1,4,2-dithiazine, 1,4,2-dioxazine, 2H-1,3,4-oxadiazine, 2H-1,3,5-oxadiazine, 6H-1,2,5-oxadiazine, 4H-1,3,4-oxadiazine, 4H-1,3,5-oxadiazine, 4H-1,2,5-oxadiazine, 2H-1,3,5-thiadiazine, 6H-1,2,5-thiadiazine, 4H-1,3,4-thiadiazine, 4H-1,3,5-thiadiazine, 4H-1,2,5-thiadiazine, 2H-1,3,4-thiadiazine, 6H-1,3,4-thiadiazine, 6H-1,3,4-oxadiazine and 1,4,2-oxathiazine, wherein the heterocycle is optionally vicinally fused with a saturated or unsaturated 5-, 6- or 7-membered ring containing 0, 1 or 2 atoms independently selected from N, O and S;
R⁸ is hydrogen, hydroxyl, halogen, thiol, nitro, alkyl, alkenyl, alkynyl, aryl, alkoxy, alkylthio, alkylsulfinyl, alkylsulfonyl, alkylamino, amino, arylalkenyl, arylalkynyl, acyl, aminoalkyl, heterocyclic, thionitroso, or —(CH₂)₀₋₃(NR^{8c})₀₋₁C(=E′)ER^{8a};
E is CR^{8d}R^{8e}, S, NR^{8b} or O;
E′ is O, NR^{8f} or S;
R⁹ is hydrogen, hydroxyl, halogen, thiol, nitro, alkyl, alkenyl, alkynyl, aryl, alkoxy, alkylthio, alkylsulfinyl, alkylsulfonyl, arylalkyl, amido, carboxylate, aminocarbonyl, arylalkenyl, arylalkynyl, acyl, aminoalkyl, heterocyclic, thionitroso, or —(CH₂)₀₋₃(NR^{9c})₀₋₁C(=Z′)ZR^{9a};
Z is CR^{9d}R^{9e}, S, NR^{9b} or O;
Z′ is O, S, or NR^{9f};
R^{8a}, R^{8b}, R^{8c}, R^{8d}, R^{8e}, R^{9a}, R^{9b}, R^{9c}, R^{9d}, R^{9e}, and R^{9f} are each independently hydrogen, acyl, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R¹⁰ is hydrogen;
R¹³ is hydrogen, hydroxy, alkyl, alkenyl, alkynyl, alkoxy, alkylthio, aryl, alkylsulfinyl, alkylsulfonyl, alkylamino, or an arylalkyl;
Y′ and Y are each independently hydrogen, halogen, hydroxyl, cyano, sulfhydryl, amino, alkyl, alkenyl, alkynyl, alkoxy, alkylthio, alkylsulfinyl, alkylsulfonyl, alkylamino, or an arylalkyl;
and pharmaceutically acceptable salts, esters and enantiomers thereof.

In one embodiment, the tetracycline compound of formula (I), (II), (III), (IV) or (V) is 10-substituted sancycline, wherein R², R^{2′}, R³, R¹¹, and R¹² are each hydrogen or a prodrug moiety; R⁴ is NR^{4a}R^{4b}; R^{4a} and R^{4b} are each alkyl; X is CR⁶R^{6′}; and R^{4′}, R⁵, R^{5′}, R⁶, and R^{6′} are each hydrogen.

In another embodiment, the tetracycline compound of formula (I), (II), (III), (IV) or (V) is 10-substituted tetracycline, wherein R², R^{2′}, R³, R¹¹, and R¹² are each hydrogen or a prodrug moiety; R⁴ is NR^{4a}R^{4b}; R^{4a} and R^{4b} are each alkyl; R^{4′}, R⁵ and R^{5′} are hydrogen and X is CR⁶R^{6′}, wherein R⁶ is methyl and R^{6′} is hydroxy.

In another embodiment, the tetracycline compound of formula (I), (II), (III), (IV) or (V) is 10-substituted doxycycline, wherein R², R^{2′}, R³, R¹¹, and R¹² are each hydrogen or a prodrug moiety; R⁴ is NR^{4a}R^{4b}; R^{4a} and R^{4b} are each alkyl; R⁵ is hydroxyl; X is CR⁶R^{6′}; R⁶ is methyl; and R^{4′}, R^{5′} and R^{6′} are hydrogen.

In another embodiment, the tetracycline compound of formula (I), (II), (III), (IV) or (V) is 10-substituted minocycline, wherein R², R^{2′}, R³, R¹¹, and R¹² are each hydrogen or a prodrug moiety; R⁴ is NR^{4a}R^{4b}; R^{4a} and R^{4b} are each alkyl; X is CR⁶R^{6′}; R^{4′}, R⁵, R^{5′}, R⁶ and R^{6′} are hydrogen atoms and R⁷ is dimethylamino.

In one embodiment, R¹⁰ is hydrogen. In another embodiment, R¹⁰ is a halogen (e.g., fluorine, bromine, chlorine, iodine, etc.), alkylcarbonyloxy, arylcarbonyloxy, alkoxycarbonyloxy, aryloxycarbonyloxy, carboxylate, alkylcarbonyl, alkylaminocarbonyl, arylalkyl aminocarbonyl, alkenylaminocarbonyl, alkylcarbonyl, arylcarbonyl, arylalkylcarbonyl, alkenylcarbonyl, alkoxycarbonyl, silyl, aminocarbonyl, alkylthiocarbonyl, phosphate, phosphonato, phosphinato, cyano, amino, acylamino, amidino, imino, sulfhydryl, alkylthio, arylthio, thiocarboxylate, sulfate, alkylsulfinyl, sulfonato, sulfamoyl, sulfonamido, nitro, trifluoromethyl, cyano, azido, alkenyl, heterocyclyl, alkylaryl, aryl and heteroaryl.

In one embodiment, the 10-substituted tetracycline compounds do not include 4-dimethylamino-3,12,12a-trihydroxy-1,11-dioxo-10-propoxy-1,4,4a,5,5 a,6,11,12a-octahydro-naphthacene-2-carboxylic acid amide; 10-butoxy-4-dimethylamino-3,12,12a-trihydroxy-1,11-dioxo-1,4,4a,5,5a,6,11,12a-octahydro-naphthacene-2-carboxylic acid amide; 10-butoxy-4,7-bis-dimethylamino-3,12,12a-trihydroxy-1,11-dioxo-1,4,4a,5,5a,6,11,12-octahydro-naphthacene-2-carboxylic acid amide; 3,10-bis-benzyloxy-4,7-bis-dimethylamino-12,12a-dihydroxy-1,11-dioxo-1,4,4a,5,5a,6,11,12a-octahydro-naphthacene-2-carboxylic acid amide; 10-butyloxy-4-dimethylamino-3,12-12a-trihydroxy-1,11-dioxo-7-(4-trifluoromethyl-phenyl)-1,4,4a,5,5a,6,11,12a-octahydro-naphthacene-carboxylic acid; benzenesulfonic acid 9-cyano-7-dimethylamino-8,10a,11-trihydroxy-10,12-dioxo-5,5a,6,6a,7,10,10a,12-octahydro-naphthacen-1-yl ester; 10-butoxy-4-dimethylamino-7-(4-dimethylamino-phenyl)-3,12,12a-trihydroxy-1,11-dioxo-1,4,4a,5,5a,6,11,12a-octahydro-naphthacene-2-carboxylic acid amide; 4-dimethylamino-3,12,12a-trihydroxy-10-(3-hydroxy-propoxy)-1,11-dioxo-1,4,4a,5,5a,6,11,12a-octahydro-naphthacene-2-carboxylic acid amide; 10-butoxy-4-dimethylamino-3,5,12,12a-tetrahydroxy-6-methyl-1,11-dioxo-1,4,4a,5,5a,6,11,12a-octahydro-naphthacene-2-carboxylic acid amide; 3,10-bis-allyloxy-4-dimethylamino-12,12a-dihydroxy-1,11-dioxo-1,4,4a,5,5a,6,11,12a-octahydro-naphthacene-2-carboxylic acid amide; 3,10-bis-benzyloxy-4-dimethylamino-12,12a-dihydroxy-1,11-dioxo-1,4,4a,5,5a,6,11,12a-octahydro-naphthacene-2-carboxylic acid amide; or 10-butoxy-4-dimethylamino-3,12,12a-trihydroxy-7-iodo-1,11-dioxo-1,4,4a,5,5a,6,11,12a-octahydro-naphthacene-2-carboxylic acid amide.

In one embodiment, R¹⁰ is substituted or unsubstituted alkyl, e.g., methyl, ethyl, propyl, butyl, pentyl, etc. Examples of substituents include but are not limited to halogens (e.g., fluorine, bromine, chlorine, iodine, etc.), alkylcarbonyloxy, arylcarbonyloxy, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, arylalkylcarbonyl, alkenylcarbonyl, alkoxycarbonyl, silyl, aminocarbonyl, alkylthiocarbonyl, phosphate, phosphonato, phosphinato, cyano, amino, acylamino, amidino, imino, sulfhydryl, alkylthio, arylthio, thiocarboxylate, sulfate, alkylsulfinyl, sulfonato, sulfamoyl, sulfonamido, nitro, trifluoromethyl, cyano, azido, alkenyl, heterocyclyl, alkylaryl, aryl and heteroaryl.

In one embodiment, R¹⁰ can be substituted or unsubstituted alkenyl. Examples of substituents for alkenyl R¹⁰ groups include those listed above for alkyl R¹⁰ groups and can also include hydroxyl and alkoxy (e.g., methoxy, ethoxy, propoxy, perfluoromethoxy, perchloromethoxy, etc.). In another embodiment, R¹⁰ can be substituted or unsubstituted alkynyl. Examples of substituents for alkenyl R¹⁰ groups include those listed above for alkyl R¹⁰ groups and alkenyl R¹⁰ groups.

In a further embodiment, R¹⁰ can be an aryl moiety such as substituted and unsubstituted phenyl. Examples of possible substituents of aryl R¹⁰ groups include, but are not limited to, alkyl (e.g., methyl, ethyl, propyl, butyl, pentyl, hexyl, perfluormethyl, perchloroethyl, etc.), alkenyl, halogen (e.g., fluorine, chlorine, bromine, iodine, etc.), hydroxyl, alkoxy (e.g., methoxy, ethoxy, propoxy, perfluoromethoxy, perchloromethoxy, etc.), alkylcarbonyloxy, arylcarbonyloxy, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, arylalkylcarbonyl, alkenylcarbonyl, alkoxycarbonyl, silyl, aminocarbonyl, alkylthiocarbonyl, phosphate, phosphonato, phosphinato, cyano, amino, acylamino, amido, imino, sulfhydryl, alkylthio, arylthio, thiocarboxylate, sulfate, alkylsulfinyl, sulfonato, sulfamoyl, sulfonamido, nitro, acetyl, alkyl, cyano, azido, heterocyclyl, alkylaryl, aryl and heteroaryl groups.

Other examples of aryl R¹⁰ groups include substituted and unsubstituted heterocycles (e.g., furanyl, imidazolyl, benzothiophenyl, benzofuranyl, quinolinyl, isoquinolinyl, benzodioxazolyl, benzoxazolyl, benzothiazolyl, benzoimidazolyl, methylenedioxyphenyl, indolyl, thienyl, pyrimidyl, pyrazinyl, purinyl, pyrazolyl, pyrolidinyl, oxazolyl, isooxazolyl, naphthridinyl, thiazolyl, isothiazolyl, or deazapurinyl) and substituted and unsubstituted biaryl groups, such as naphthyl and fluorene.

In yet another embodiment, R¹⁰ can be a substituted or unsubstituted amino. Examples of suitable amino R¹⁰ moieties include, for example, amino, alkylamino, dialkylamino, arylamino, diarylamino, and cyclodialkylamino. In one embodiment, the amino group may be heterocyclic, e.g., substituted or unsubstituted piperidine. In another embodiment, the amino group is 4-methyl piperidine.

In one embodiment, R¹⁰ is substituted or unsubstituted sulfonyl. Suitable sulfonyl groups can include substituted or unsubstituted alkylsulfonyl, such as trifluromethylsulfonyl or methylsulfonyl, and substituted or unsubstituted arylsulfonyl, such as phenylsulfonyl and para-toluenesulfonyl.

In another embodiment, R¹⁰ is acyl.

In a further embodiment, R⁹ is hydrogen. In another embodiment, R⁹ is substituted or unsubstituted aryl (e.g., substituted or unsubstituted carbocyclic, e.g., phenyl or naphthyl; or substituted or unsubstituted heteroaryl). R⁹ also may be substituted or unsubstituted alkenyl or substituted or unsubstituted alkynyl. R⁹ also may be heterocyclic or alkylcarbonyl, alkenylcarbonyl, alkynylcarbonyl, alkyloxycarbonyl, or otherwise comprise a substituted carbonyl, acyl, acetyl, or formyl moiety.

In another further embodiment, R⁹ is substituted or unsubstituted alkyl. In a further embodiment, R⁹ is aminoalkyl, e.g., aminomethyl. In a further embodiment, the aminoalkyl is further substituted with any substituent which allows the compound to perform its intended function. In a further embodiment, the aminoalkyl substituent is alkylaminomethyl.

In another embodiment, R⁹ is substituted or unsubstituted amino, e.g., alkylamino, dialkylamino, arylamino, alkylcarbonylamino, alkylaminocarbonyl amino, arylcarbonylamino, etc. In another embodiment, R⁹ is amido. In yet another embodiment, R⁹ is cyano, halogen (e.g., fluorine, bromine, chlorine, iodo, etc.), nitro, hydroxyl, alkoxy, or any other substituent which allows the tetracycline compound to perform its intended function. In another embodiment, R⁹ is an R⁹ moiety described in WO 03/079984; WO 03/075857; WO 02/04406; or WO 01/74761, incorporated herein by reference in its entirety.

In another embodiment, the tetracycline compound of the invention is a compound wherein R⁹ is —NR^{9c}C(=Z′)ZR^{9a}, —CH₂NR^{9c}C(=Z′)ZR^{9a}, —(CH₂)₂NR^{9c}C(=Z′)ZR^{9a}, or —(CH₂)₃NR^{9c}C(=Z′)ZR^{9a}. In certain embodiments, R⁹ is —NR^{9c}C(=Z′)ZR^{9a} or —CH₂NR^{9c}C(=Z′)ZR^{9a}. Examples of R^{9c} include hydrogen. Z′ may be, for example, S, NH, or O. Examples of Z include NR^{9b} (e.g., when R^{9b} is hydrogen, alkyl, etc.), O or S.

Examples of R^{9a} groups include aryl groups such as substituted and unsubstituted phenyl. Examples of possible substituents of aryl R^{9a} groups include, but are not limited to, alkyl (e.g., methyl, ethyl, propyl, butyl, pentyl, hexyl, perfluormethyl, perchloroethyl, etc.), alkenyl, halogen (e.g., fluorine, chlorine, bromine, iodine, etc.), hydroxyl, alkoxy (e.g., methoxy, ethoxy, propoxy, perfluoromethoxy, perchloromethoxy, etc.), alkylcarbonyloxy, arylcarbonyloxy, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, arylalkylcarbonyl, alkenylcarbonyl, alkoxycarbonyl, silyl, aminocarbonyl, alkylthiocarbonyl, phosphate, phosphonato, phosphinato, cyano, amino, acylamino, amido, imino, sulfhydryl, alkylthio, arylthio, thiocarboxylate, sulfate, alkylsulfinyl, sulfonato, sulfamoyl, sulfonamido, nitro, acetyl, alkyl, cyano, azido, heterocyclyl, alkylaryl, aryl and heteroaryl groups.

In certain embodiments, at least one of the substituents of the substituted phenyl is nitro, alkoxy (e.g., methoxy, methylenedioxy, perfluoromethoxy) alkyl (e.g., methyl, ethyl, propyl, butyl, or pentyl), acetyl, halogen (e.g., fluorine, chlorine, bromine, or iodine), or amino (e.g., dialkylamino). In certain embodiments, the alkoxy group is perhalogenated, e.g., perfluoromethoxy.

Examples of aryl R^{9a} groups include, but are not limited to, unsubstituted phenyl, para-nitrophenyl, para-methoxy phenyl, para-perfluoromethoxy phenyl, para-acetyl phenyl, 3,5-methylenedioxyphenyl, 3,5-diperfluoromethyl phenyl, para-bromo phenyl, para-chloro phenyl, and para-fluoro phenyl.

Other examples of aryl R^{9a} groups include substituted and unsubstituted heterocycles (e.g., furanyl, imidazolyl, benzothiophenyl, benzofuranyl, quinolinyl, isoquinolinyl, benzodioxazolyl, benzoxazolyl, benzothiazolyl, benzoimidazolyl, methylenedioxyphenyl, indolyl, thienyl, pyrimidyl, pyrazinyl, purinyl, pyrazolyl, pyrolidinyl, oxazolyl, isooxazolyl, naphthridinyl, thiazolyl, isothiazolyl, or deazapurinyl) and substituted and unsubstituted biaryl groups, such as naphthyl and fluorene.

R^{9a} also may be substituted or unsubstituted alkyl, e.g., methyl, ethyl, propyl, butyl, pentyl, etc. Examples of substituents include but are not limited to halogens (e.g., fluorine, bromine, chlorine, iodine, etc.), hydroxyl, alkoxy (e.g., methoxy, ethoxy, propoxy, butoxy, etc.), alkylcarbonyloxy, arylcarbonyloxy, alkoxycarbonyloxy, aryloxycarbonyloxy, carboxylate, alkylc arbonyl, alkylaminoacarbonyl, arylalkyl aminocarbonyl, alkenylaminocarbonyl, alkylcarbonyl, arylcarbonyl, arylalkylcarbonyl, alkenylcarbonyl, alkoxycarbonyl, silyl, aminocarbonyl, alkylthiocarbonyl, phosphate, phosphonato, phosphinato, cyano, amino, acylamino, amidino, imino, sulfhydryl, alkylthib, arylthio, thiocarboxylate, sulfate, alkylsulfinyl, sulfonato, sulfamoyl, sulfonamido, nitro, trifluoromethyl, cyano, azido, alkenyl, heterocyclyl, alkylaryl, aryl and heteroaryl.

R^{9a} also can be substituted or unsubstituted alkenyl. Examples of substituents for alkenyl R^{9a} groups include those listed above for alkyl R^{9a} groups. Examples of alkenyl R^{9a} groups include pent-1-enyl.

In an embodiment, Z′ is NH, Z is NH, and R^{9a} is alkyl.

In another embodiment, R⁹ is alkyl and substituted with a heterocycle, such as 2,3-dihydro-isoindole.

In another embodiment, R⁹ is —C(=Z′)R^{9a}, Z′ is NR^{9f}, and R^{9a} is hydrogen. R^{9f} may be alkoxy.

In a further embodiment, R⁹ is substituted aminoalkyl. R⁹ may be substituted, for example, with a substituted or unsubstituted alkyloxycarbonyl group, a substituted or unsubstituted alkyl group and/or a substituted or unsubstituted aralkyl group.

In a further embodiment, R⁷ is hydrogen. In another embodiment, R⁷ is substituted or unsubstituted aryl (e.g., substituted or unsubstituted carbocyclic, e.g., phenyl or naphthyl; or substituted or unsubstituted heteroaryl). R⁷ also may be substituted or unsubstituted alkenyl or substituted or unsubstituted alkynyl. R⁷ also may be heterocyclic or alkylcarbonyl, alkenylcarbonyl, alkynylcarbonyl, alkyloxycarbonyl, or otherwise comprise a substituted carbonyl, acyl, acetyl, or formyl moiety.

In another further embodiment, R⁷ is substituted or unsubstituted alkyl. In a further embodiment, R⁷ is aminoalkyl, e.g., aminomethyl. In a further embodiment, the aminoalkyl is further substituted with any substituent which allows the compound to perform its intended function. In a further embodiment, the aminoalkyl substituent is alkylaminomethyl.

In another embodiment, R⁷ is substituted or unsubstituted amino, e.g., alkylamino, dialkylamino, arylamino, alkyl carbonylamino, alkyl aminocarbonylamino, arylcarbonylamino, etc. In another embodiment, R⁷ is amido. In yet another embodiment, R⁷ is cyano, halogen (e.g., fluorine, bromine, chlorine, iodo, etc.), nitro, hydroxyl, alkoxy, acyl, or any other substituent which allows the tetracycline compound to perform its intended function. In another embodiment, R⁷ is a 7-position moiety described in WO 02/04407, WO 01/74761, WO 03/079984, or WO 03/075857, incorporated herein by reference in their entirety.

In one embodiment, R^{7g} and R^{7h} are joined together to form a substituted 6-membered ring.

In another embodiment, R⁷ⁱ is pyrimadine.

In a further embodiment, R⁸ is hydrogen. In another embodiment, R⁸ is substituted or unsubstituted aryl (e.g., substituted or unsubstituted carbocyclic, e.g., phenyl or naphthyl; or substituted or unsubstituted heteroaryl). R⁸ also may be substituted or unsubstituted alkenyl or substituted or unsubstituted alkynyl. R⁸ also may be heterocyclic or alkylcarbonyl, alkenylcarbonyl, alkynylcarbonyl, alkyloxycarbonyl, or otherwise comprise a substituted carbonyl, acyl, acetyl, or formyl moiety.

In another further embodiment, R⁸ is substituted or unsubstituted alkyl. In a further embodiment, R⁸ is aminoalkyl, e.g., aminomethyl. In a further embodiment, the aminoalkyl is further substituted with any substituent which allows the compound to perform its intended function. In a further embodiment, the aminoalkyl substituent is alkylaminomethyl.

In another embodiment, R⁸ is substituted or unsubstituted amino, e.g., alkylamino, dialkylamino, arylamino, alkyl carbonylamino, alkylaminocarbonyl amino, arylcarbonylamino, etc. In another embodiment, R⁸ is amido. In yet another embodiment, R⁸ is cyano, halogen (e.g., fluorine, bromine, chlorine, iodo, etc.), nitro, hydroxyl, alkoxy, or any other substituent which allows the tetracycline compound to perform its intended function. In another embodiment, R⁸ is an R⁸ moiety described in WO 02/12170, WO 02/04404, or WO 03/079984, incorporated herein by reference in their entirety.

In another embodiment, R², R^{2′} R³, R¹¹, and R¹² are each independently hydrogen, alkyl, acyl, aryl, or arylalkyl. R², R^{2′} R³, R¹¹, and R¹² moieties are described in U.S. Ser. No. 10/619,653, incorporated herein by reference in its entirety. Other examples of R² and R^{2′} moieties are described in U.S. Published Application 20040002481. 26.

In another embodiment, the invention pertains to tetracycline compounds of formula II, wherein Q is a single bond. When Q is a single bond, the invention pertains to tetracycline compounds wherein J is CR^{21a}R^{21b}, O, S, or NR^{21c} and K is CR^{22a}R^{22b}, O, S, or NR^{22c}.

In yet another embodiment, the invention pertains to tetracycline compounds of formula II, wherein Q is a double bond. When Q is a double bond, the invention pertains to tetracycline compounds wherein J is CR^{21a}R^{21b}, K is CR^{22a}R^{22b} and R^{21b} and R^{22b} are absent; J is NR^{21c}, K is CR^{22a}R^{22b} and R^{21c} and R^{22b} are absent; or J is CR^{21a}R^{21b}, K is NR^{22c}, and R^{21b} and R^{22c} are absent.

In one embodiment, R^{21a}, R^{21b}, R^{21c}, R^{22a}, R^{22b}, and R^{22c} are each independently hydrogen. In another embodiment, R^{21a}, R^{21b}, R^{21c}, R^{22a}, R^{22b}, and R^{22c} are each independently halogens (e.g., fluorine, bromine, chlorine, iodine, etc.), hydroxyl, alkoxy (e.g., methoxy, ethoxy, propoxy, butoxy, etc.), alkylcarbonyloxy, arylcarbonyloxy, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, arylalkylcarbonyl, alkenylcarbonyl, alkoxycarbonyl, silyl, aminocarbonyl, alkylthiocarbonyl, phosphate, phosphonato, phosphinato, cyano, amino, acylamino, amidino, imino, sulfhydryl, alkylthio, arylthio, thiocarboxylate, sulfate, alkylsulfinyl, sulfonato, sulfamoyl, sulfonamido, nitro, trifluoromethyl, cyano, azido, alkenyl, heterocyclyl, alkylaryl, aryl and heteroaryl.

In another embodiment, R^{21a}, R^{21b}, R^{21c}, R^{22a}, R^{22b}, and R^{22c} are each independently substituted or unsubstituted alkyl, e.g., methyl, ethyl, propyl, butyl, pentyl, etc. Examples of substituents include but are not limited to halogens (e.g., fluorine, bromine, chlorine, iodine, etc.), hydroxyl, alkoxy (e.g., methoxy, ethoxy, propoxy, butoxy, etc.), alkylcarbonyloxy, arylcarbonyloxy, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, arylalkylcarbonyl, alkenylcarbonyl, alkoxycarbonyl, silyl, aminocarbonyl, alkylthiocarbonyl, phosphate, phosphonato, phosphinato, cyano, amino, acylamino, amidino, imino, sulflhydryl, alkylthio, arylthio, thiocarboxylate, sulfate, alkylsulfinyl, sulfonato, sulfamoyl, sulfonamido, nitro, trifluoromethyl, cyano, azido, alkenyl, heterocyclyl, alkylaryl, aryl and heteroaryl.

Examples of substituted alkyl R^{21a}, R^{21b}, R^{21c}, R^{22a}, R^{22b}, and R^{22c} groups include, but are not limited to, alkylsilyl, such as trimethylsilyl.

In another embodiment, R^{21a}, R^{21b}, R^{21c}, R^{22a}, R^{22b}, and R^{22c} are each independently substituted or unsubstituted alkenyl. Examples of substituents for alkenyl R¹⁰ groups include those listed above for alkyl R¹⁰ groups. In another embodiment, R^{21a}, R^{21b}, R^{21c}, R^{22a}, R^{22b}, and R^{22c} are each independently substituted or unsubstituted alkynyl. Examples of substituents for alkenyl R¹⁰ groups include those listed above for alkyl R¹⁰ groups.

In a further embodiment, R^{21a}, R^{21b}, R^{21c}, R^{22a}, R^{22b}, and R^{22c} are each independently an aryl moiety such as substituted and unsubstituted phenyl. Examples of possible substituents of aryl R^{21a}, R^{21b}, R^{21c}, R^{22a}, R^{22b}, and R^{22c} groups include, but are not limited to, alkyl (e.g., methyl, ethyl, propyl, butyl, pentyl, hexyl, perfluormethyl, perchloroethyl, etc.), alkenyl, halogen (e.g., fluorine, chlorine, bromine, iodine, etc.), hydroxyl, alkoxy (e.g., methoxy, ethoxy, propoxy, perfluoromethoxy, perchloromethoxy, etc.), alkylcarbonyloxy, arylcarbonyloxy, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, arylalkylcarbonyl, alkenylcarbonyl, alkoxycarbonyl, silyl, aminocarbonyl, alkylthiocarbonyl, phosphate, phosphonato, phosphinato, cyano, amino, acylamino, amido, imino, sulfhydryl, alkylthio, arylthio, thiocarboxylate, sulfate, alkylsulfinyl, sulfonato, sulfamoyl, sulfonamido, nitro, acetyl, alkyl, cyano, azido, heterocyclyl, alkylaryl, aryl and heteroaryl groups.

Examples of aryl R^{21a}, R^{21b}, R^{21c}, R^{22a}, R^{22b}, and R^{22c} groups include, but are not limited to, unsubstituted phenyl and para-amino substituted phenyl.

Other examples of aryl R^{21a}, R^{21b}, R^{21c}, R^{22a}, R^{22b}, and R^{22c} groups include substituted and unsubstituted heterocycles (e.g., furanyl, imidazolyl, benzothiophenyl, benzofuranyl, quinolinyl, isoquinolinyl, benzodioxazolyl, benzoxazolyl, benzothiazolyl, benzoimidazolyl, methylenedioxyphenyl, indolyl, thienyl, pyrimidyl, pyrazinyl, purinyl, pyrazolyl, pyrolidinyl, oxazolyl, isooxazolyl, naphthridinyl, thiazolyl, isothiazolyl, or deazapurinyl) and substituted and unsubstituted biaryl groups, such as naphthyl and fluorene.

In another embodiment, the invention pertains to tetracycline compounds of formula III, wherein G is CR^{23a}R^{23b}, S, or NR^{23c}; L is CR^{24a}R^{24b}, S, or NR^{24c}; M is CR^{25a}R^{25b}, C=T, O, S, or NR^{25c}; and T is O, S or NR^{25d};

In one embodiment, R^{23a}, R^{23b}, R^{23c}, R^{24a}, R^{24b}, R^{24c}, R^{25a}, R^{25b}, R^{25c}, and R^{25d} are each independently hydrogen. In another embodiment, R^{23a}, R^{23b}, R^{23c}, R^{24a}, R^{24b}, R^{24c}, R^{25a}, R^{25b}, R^{25c}, and R^{25d} are each independently halogens (e.g., fluorine, bromine, chlorine, iodine, etc.), hydroxyl, alkoxy (e.g., methoxy, ethoxy, propoxy, butoxy, etc.), alkylcarbonyloxy, arylcarbonyloxy, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, arylalkylcarbonyl, alkenylcarbonyl, alkoxycarbonyl, silyl, aminocarbonyl, alkylthiocarbonyl, phosphate, phosphonato, phosphinato, cyano, amino, acylamino, amidino, imino, sulfhydryl, alkylthio, arylthio, thiocarboxylate, sulfate, alkylsulfinyl, sulfonato, sulfamoyl, sulfonamido, nitro, trifluoromethyl, cyano, azido, alkenyl, heterocyclyl, alkylaryl, aryl and heteroaryl.

In another embodiment, R^{23a}, R^{23b}, R^{23c}, R^{24a}, R^{24b}, R^{24c}, R^{25a}, R^{25b}, R^{25c}, and R^{25d} are each independently substituted or unsubstituted alkyl, e.g., methyl, ethyl, propyl, butyl, pentyl, etc. Examples of substituents include but are not limited to halogens (e.g., fluorine, bromine, chlorine, iodine, etc.), hydroxyl, alkoxy (e.g., methoxy, ethoxy, propoxy, butoxy, etc.), alkylcarbonyloxy, arylcarbonyloxy, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, arylalkylcarbonyl, alkenylcarbonyl, alkoxycarbonyl, silyl, aminocarbonyl, alkylthiocarbonyl, phosphate, phosphonato, phosphinato, cyano, amino, acylamino, amidino, imino, sulfhydryl, alkylthio, arylthio, thiocarboxylate, sulfate, alkylsulfinyl, sulfonato, sulfamoyl, sulfonamido, nitro, trifluoromethyl, cyano, azido, alkenyl, heterocyclyl, alkylaryl, aryl and heteroaryl.

In another embodiment, R^{23a}, R^{23b}, R^{23c}, R^{24a}, R^{24b}, R^{24c}, R^{25a}, R^{25b}, R^{25c}, and R^{25d} are each independently substituted or unsubstituted alkenyl. Examples of substituents for alkenyl R^{23a}, R^{23b}, R^{23c}, R^{24a}, R^{24b}, R^{24c}, R^{25a}, R^{25b}, R^{25c} and R^{25d} groups include those listed above for alkyl R¹⁰ groups. In another embodiment, R^{23a}, R^{23b}, R^{23c}, R^{24a}, R^{24b}, R^{24c}, R^{25a}, R^{25b}, R^{25c}, and R^{25d} are each independently substituted or unsubstituted alkynyl. Examples of substituents for alkenyl R^{23a}, R^{23b}, R^{23c}, R^{24a}, R^{24b}, R^{24c}, R^{25a}, R^{25b}, R^{25c}, and R^{25c} and R^{25d} groups include those listed above for alkyl R¹⁰ groups.

In a further embodiment, R^{23a}, R^{23b}, R^{23c}, R^{24a}, R^{24b}, R^{24c}, R^{25a}, R^{25b}, R^{25c}, and R^{25d} are each independently an aryl moiety such as substituted and unsubstituted phenyl. Examples of possible substituents of aryl R^{23a}, R^{23b}, R^{23c}, R^{24a}, R^{24b}, R^{24c}, R^{25a}, R^{25b}, R^{25c}, and R^{25d} groups include, but are not limited to, alkyl (e.g., methyl, ethyl, propyl, butyl, pentyl, hexyl, perfluormethyl, perchloroethyl, etc.), alkenyl, halogen (e.g., fluorine, chlorine, bromine, iodine, etc.), hydroxyl, alkoxy (e.g., methoxy, ethoxy, propoxy, perfluoromethoxy, perchloromethoxy, etc.), alkylcarbonyloxy, arylcarbonyloxy, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, arylalkylcarbonyl, alkenylcarbonyl, alkoxycarbonyl, silyl, aminocarbonyl, alkylthiocarbonyl, phosphate, phosphonato, phosphinato, cyano, amino, acylamino, amido, imino, sulfhydryl, alkylthio, arylthio, thiocarboxylate, sulfate, alkylsulfinyl, sulfonato, sulfamoyl, sulfonamido, nitro, acetyl, alkyl, cyano, azido, heterocyclyl, alkylaryl, aryl and heteroaryl groups.

Other examples of aryl R^{23a}, R^{23b}, R^{23c}, R^{24a}, R^{24b}, R^{24c}, R^{25a}, R^{25b}, R^{25c} and R^{25d} groups include substituted and unsubstituted heterocycles (e.g., furanyl, imidazolyl, benzothiophenyl, benzofuranyl, quinolinyl, isoquinolinyl, benzodioxazolyl, benzoxazolyl, benzothiazolyl, benzoimidazolyl, methylenedioxyphenyl, indolyl, thienyl, pyrimidyl, pyrazinyl, purinyl, pyrazolyl, pyrolidinyl, oxazolyl, isooxazolyl, naphthridinyl, thiazolyl, isothiazolyl, or deazapurinyl) and substituted and unsubstituted biaryl groups, such as naphthyl and fluorene.

In one embodiment, the tetracycline compound is a 10-substituted compound of the Table 2:

TABLE 2

| Code | Compound |
|---|---|
| A | 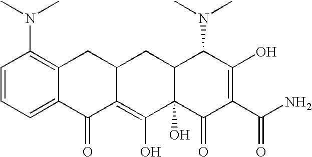 |
| C | 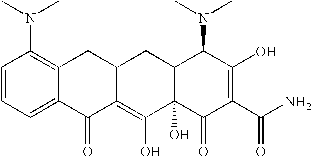 |
| D | 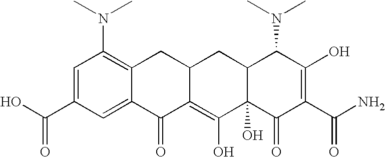 |
| E | 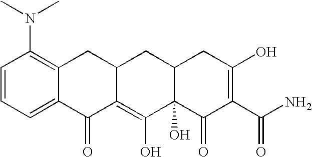 |
| F | 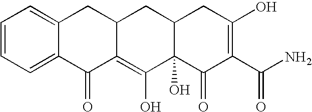 |
| G | 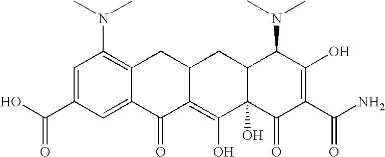 |
| H | 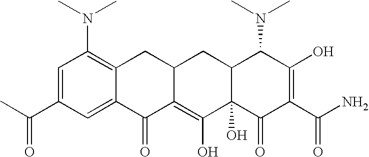 |
| I | 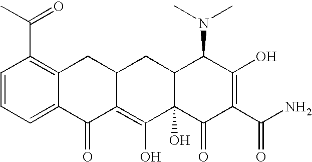 |
| J | 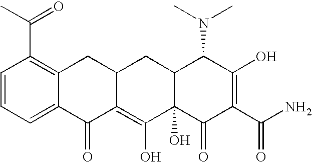 |
| K | 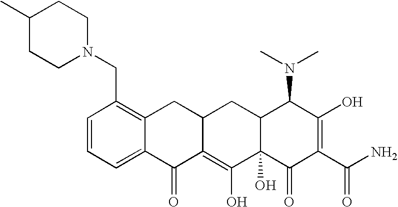 |
| L | 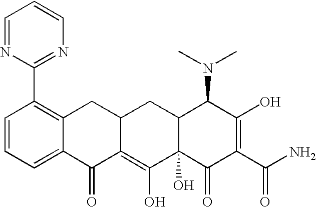 |
| M | 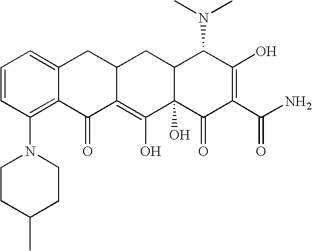 |
| N | 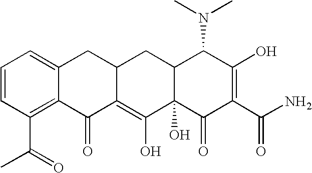 |
| O | 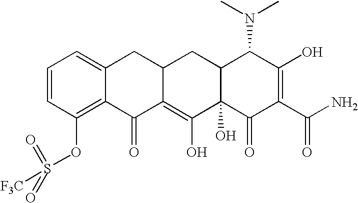 |
| P | 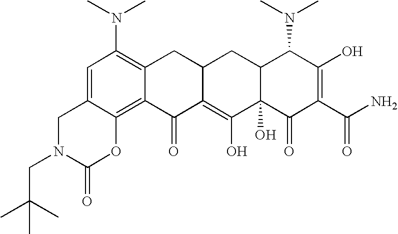 |
| Q | 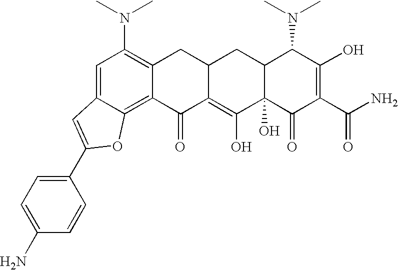 |
| R | 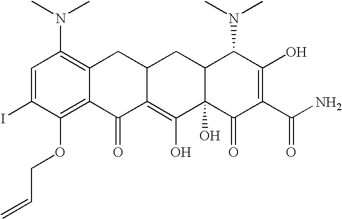 |
| S | 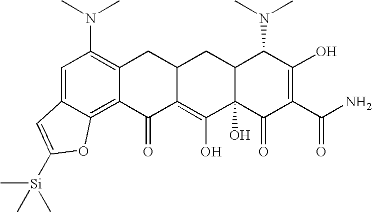 |
| T | 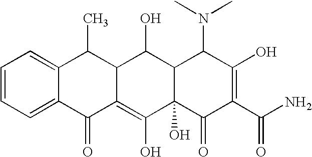 |
| U | 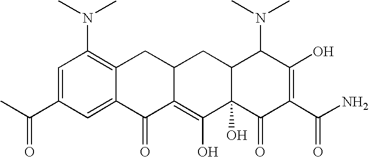 |
| V | 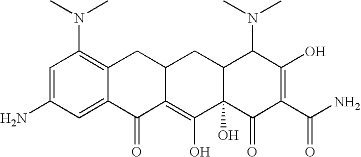 |
| W | 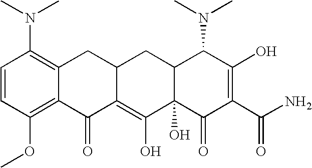 |
| X | 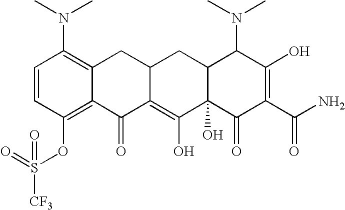 |
| Y | 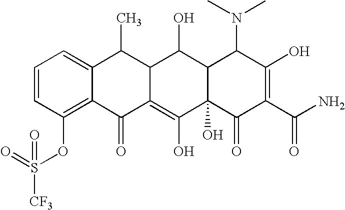 |
| Z | 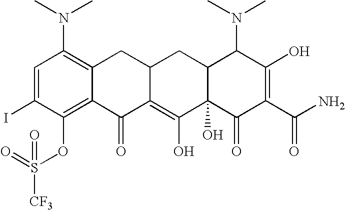 |
| AA | 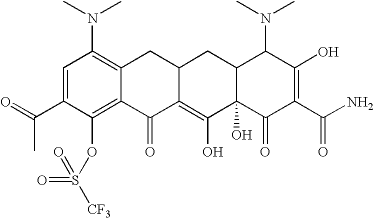 |
| AB | 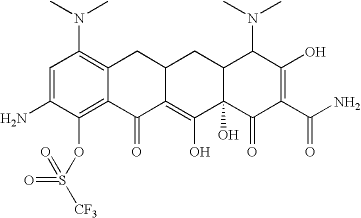 |
| AC | 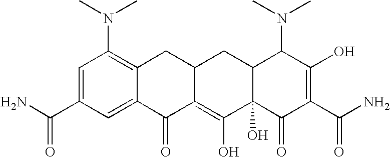 |
| AD | 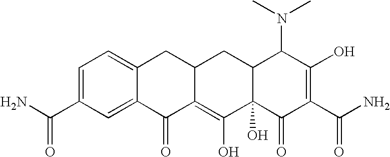 |
| AE | 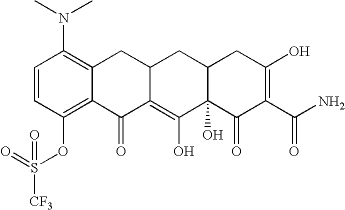 |
| AF | 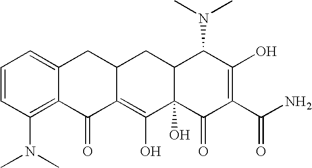 |
| AG | 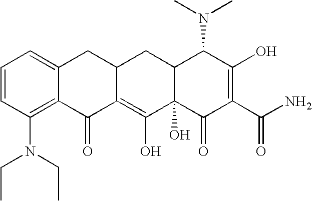 |
| AH | 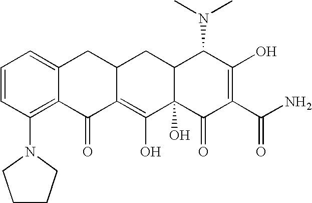 |

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone. In certain embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls have from 3-8 carbon atoms in their ring structure, and more preferably have 5 or 6 carbons in the ring structure. The term C₁-C₆ includes alkyl groups containing 1 to 6 carbon atoms.

Moreover, the term alkyl includes both “unsubstituted alkyls” and “substituted alkyls”, the latter of which refers to alkyl moieties having substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “arylalkyl” moiety is an alkyl substituted with an aryl (e.g., phenylmethyl (benzyl)). The term “alkyl” also includes the side chains of natural and unnatural amino acids.

The term “aryl” includes groups, including 5- and 6-membered single-ring aromatic groups that may include from zero to four heteroatoms, for example, benzene, phenyl, pyrrole, furan, thiophene, thiazole, isothiaozole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxophenyl, quinoline, isoquinoline, naphthridine, indole, benzofuran, purine, benzofuran, deazapurine, or indolizine. Those aryl groups having heteroatoms in the ring structure may also be referred to as “aryl heterocycles”, “heterocycles,” “heteroaryls” or “heteroaromatics”. The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, arylalkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “alkenyl” includes unsaturated aliphatic groups analogous in length and possible substitution to the alkyls described above, but that contain at least one double bond.

For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups which include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, the term alkenyl includes both “unsubstituted alkenyls” and “substituted alkenyls”, the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond.

For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups which include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, the term alkynyl includes both “unsubstituted alkynyls” and “substituted alkynyls”, the latter of which refers to alkynyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” as used herein means an alkyl group, as defined above, but having from one to five carbon atoms in its backbone structure. “Lower alkenyl” and “lower alkynyl” have chain lengths of, for example, 2-5 carbon atoms.

The term “acyl” includes compounds and moieties which contain the acyl radical (CH₃CO—) or a carbonyl group. It includes substituted acyl moieties. The term “substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acylamino” includes moieties wherein an acyl moiety is bonded to an amino group. For example, the term includes alkylcarbonylamino, arylcarbonylamino, carbamoyl and ureido groups.

The term “aroyl” includes compounds and moieties with an aryl or heteroaromatic moiety bound to a carbonyl group. Examples of aroyl groups include phenylcarboxy, naphthyl carboxy, etc.

The terms “alkoxyalkyl”, “alkylaminoalkyl” and “thioalkoxyalkyl” include alkyl groups, as described above, which further include oxygen, nitrogen or sulfur atoms replacing one or more carbons of the hydrocarbon backbone, e.g., oxygen, nitrogen or sulfur atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “amine” or “amino” includes compounds where a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group which is bound to at least one alkyl group and at least one aryl group. The term “alkaminoalkyl” or “alkyl aminoalkyl” refers to an alkyl, alkenyl, or alkynyl group bound to a nitrogen atom which is also bound to an alkyl group.

The term “amide” or “aminocarbonyl” includes compounds or moieties which contain a nitrogen atom which is bound to the carbon of a carbonyl or a thiocarbonyl group. The term includes “alkaminocarbonyl” or “alkylaminocarbonyl” groups which include alkyl, alkenyl, aryl or alkynyl groups bound to an amino group bound to a carbonyl group. It includes arylaminocarbonyl groups which include aryl or heteroaryl moieties bound to an amino group which is bound to the carbon of a carbonyl or thiocarbonyl group. The terms “alkylaminocarbonyl,” “alkenylaminocarbonyl,” “alkynylaminocarbonyl,” “arylaminocarbonyl,” “alkylcarbonylamino,” “alkenylcarbonylamino,” “alkynylcarbonylamino,” and “arylcarbonylamino” are included in term “amide.” Amides also include urea groups (aminocarbonylamino) and carbamates (oxycarbonylamino).

The term “carbonyl” or “carboxy” includes compounds and moieties which contain a carbon connected with a double bond to an oxygen atom. The carbonyl can be further substituted with any moiety which allows the compounds of the invention to perform its intended function. For example, carbonyl moieties may be substituted with alkyls, alkenyls, alkynyls, aryls, alkoxy, aminos, etc. Examples of moieties which contain a carbonyl include aldehydes, ketones, carboxylic acids, amides, esters, anhydrides, etc.

The term “thiocarbonyl” or “thiocarboxy” includes compounds and moieties which contain a carbon connected with a double bond to a sulfur atom.

The term “ether” includes compounds or moieties which contain an oxygen bonded to two different carbon atoms or heteroatoms. For example, the term includes “alkoxyalkyl” which refers to an alkyl, alkenyl, or alkynyl group covalently bonded to an oxygen atom which is covalently bonded to another alkyl group.

The term “ester” includes compounds and moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above.

The term “thioether” includes compounds and moieties which contain a sulfur atom bonded to two different carbon or hetero atoms. Examples of thioethers include, but are not limited to alkthioalkyls, alkthioalkenyls, and alkthioalkynyls. The term “alkthioalkyls” include compounds with an alkyl, alkenyl, or alkynyl group bonded to a sulfur atom which is bonded to an alkyl group. Similarly, the term “alkthioalkenyls” and alkthioalkynyls” refer to compounds or moieties wherein an alkyl, alkenyl, or alkynyl group is bonded to a sulfur atom which is covalently bonded to an alkynyl group.

The term “hydroxy” or “hydroxyl” includes groups with an —OH or —O⁻.

The term “halogen” includes fluorine, bromine, chlorine, iodine, etc. The term “perhalogenated” generally refers to a moiety wherein all hydrogens are replaced by halogen atoms.

The terms “polycyclyl” or “polycyclic radical” refer to two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminoacarbonyl, arylalkylaminocarbonyl, alkenylaminocarbonyl, alkylcarbonyl, arylcarbonyl, arylalkyl carbonyl, alkenylcarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

The term “heteroatom” includes atoms of any element other than carbon or hydrogen. Preferred heteroatoms are nitrogen, oxygen, sulfur and phosphorus.

The term “prodrug moiety” includes moieties which can be metabolized in vivo to a hydroxyl group and moieties which may advantageously remain esterified in vivo. Preferably, the prodrugs moieties are metabolized in vivo by esterases or by other mechanisms to hydroxyl groups or other advantageous groups. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters.

It will be noted that the structure of some of the tetracycline compounds of this invention includes asymmetric carbon atoms. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis. Furthermore, the structures and other compounds and moieties discussed in this application also include all tautomers thereof.

2. Methods for Synthesizing 10-Substituted Tetracycline Compounds

A method for derivatizing tetracycline compounds at the 10 position has been discovered through chemical modification. In one embodiment, the 10-substituted tetracycline can be prepared via conversion of a variety of anhydrous tetracycline freebases to the tetracycline phenoxide with a base, followed by reaction with an appropriate sulfonating agent, as seen in Scheme 1.

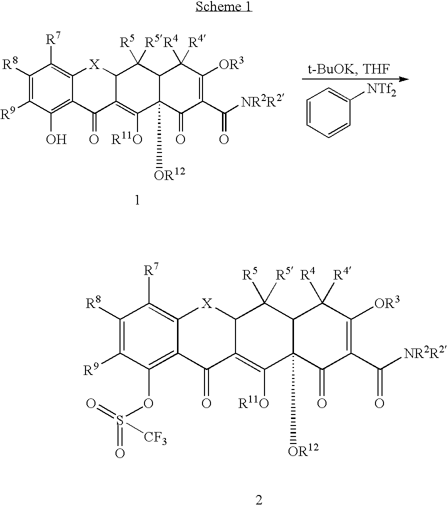

In one embodiment, the invention pertains to a method for synthesizing 10-substituted tetracycline compounds. The method includes contacting a tetracycline compound with an effective amount of a base to form a tetracycline phenoxide compound, and further contacting tetracycline phenoxide compound with a sulfonating agent, such that 10-substituted tetracycline compound is formed.

The term “base” includes agents that are able to deprotonate the C10 phenol to form the corresponding tetracycline compound. Examples of bases are described in Advanced Organic Chemistry, 4th Ed., March, pp 248-253. In one embodiment, the base is potassium tert-butoxide.

The term “sulfonating agent” includes agents that are able to transfer a sulfonyl group to the tetracycline phenoxide. Examples of sulfonating agents are described in Comprehensive Organic Transformations (“COT”) 2^{nd} Ed., Larock, pp 28-29. In one embodiment, the sulfonating agent is N-phenylbis(trifluoromethanesulfonimide).

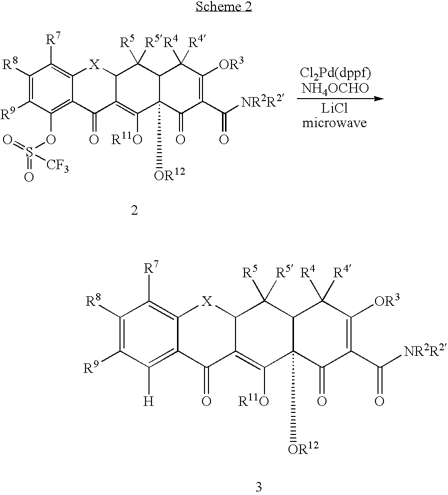

In one embodiment, the invention pertains to further modifying the sulfonated 10-substituted tetracycline compound to form 10-deoxytetracycline compounds, as seen in Scheme 2. The method includes contacting a sulfonated 10-substituted tetracycline compound with an effective amount of a reducing agent to form such that a 10-deoxytetracycline compound is formed.

The term “reducing agent” includes agents which are capable of reducing the C-10 sulfonyl to a hydrogen. Examples of reducing agents are described in Comprehensive Organic Transformations (“COT”) 2^{nd} Ed., Larock, pp 28-29. In one embodiment, the reducing agent is dichloro [1,1 bis(diphenylphosphino) ferrocene] palladium(II).

In one embodiment, the tetracycline compound is tetracycline, doxycycline, minocycline, or sancycline. In another embodiment, the tetracycline compound is a tetracycline compound described in, for example, WO 03/079983, WO 02/12170, WO 02/04407, WO 02.04406, WO 02/04405, WO 02/04404, WO 01/74761, WO 03/079984, WO 03/075857, WO 03/057169, WO 02/072545, WO 02/072506, U.S. Ser. No. 10/619,653, U.S. Ser. No. 09/895,857; U.S. Ser. No. 09/895,812; U.S. Pat. No. 5,326,759; U.S. Pat. No. 5,328,902; U.S. Pat. No. 5,495,031; U.S. Pat. No. 5,495,018; U.S. Pat. No. 5,495,030; U.S. Pat. No. 5,495,032; U.S. Pat. No. 5,512,553; U.S. Pat. No. 5,675,030; U.S. Pat. No. 5,843,925; U.S. Pat. No. 5,886,175; U.S. Pat. No. 6,165,999; U.S. Pat. No. 3,239,499; WO 95/22529; U.S. Pat. No. 5,064,821; U.S. Pat. No. 5,589,470; U.S. Pat. No. 5,811,412, or U.S. Application 20040002481.

A wide range of tetracycline compounds of the invention can be synthesized using the methods of the invention. The tetracycline compounds of the invention can be synthesized, for example, by reacting various reactive agent, with the sulfonated 10-substituted tetracyclines to produce tetracycline compounds of the invention. Examples of some of the substituted tetracyclines which can be synthesized using the methods of the invention include compounds with C10-carbon-carbon, C10-carbon-aryl or C10-carbon-heteroatom bonds as shown in Scheme 3.

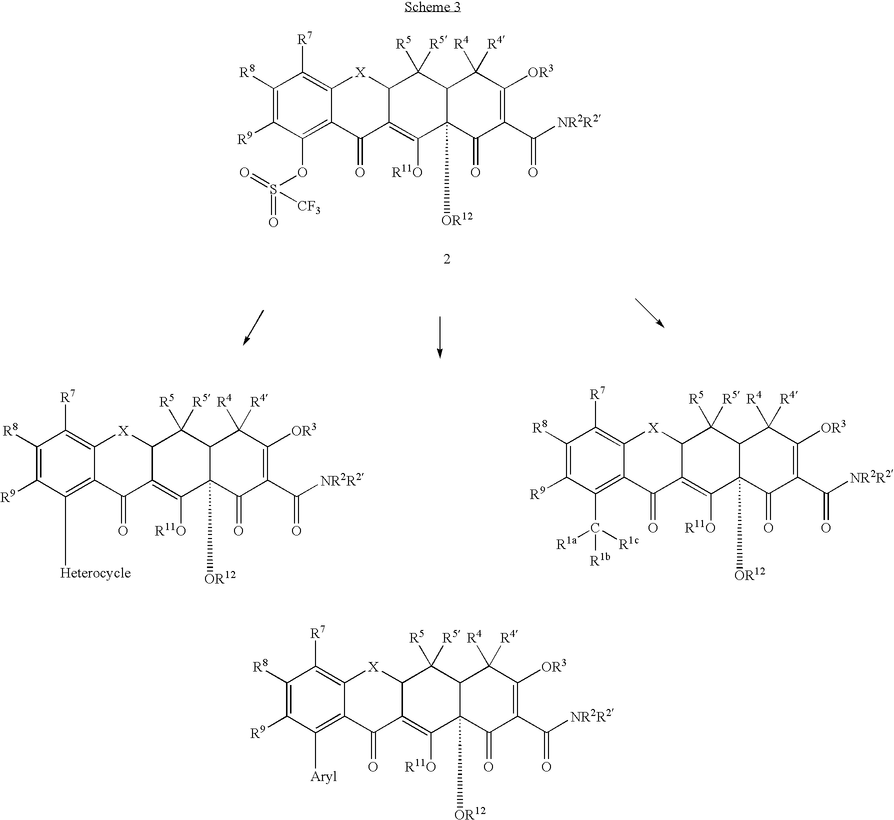

In Scheme 3, R^{1a}, R^{1b} and R^{1c} are each independently hydrogen, alkyl, heterocyclic, aryl, alkenyl, alkynyl, alkoxy, carbonyl, acyl, halogen, cyano, amino, amido, nitro, or any other substituent described herein which would allow the tetracycline compounds of the invention to perform their intended functions.

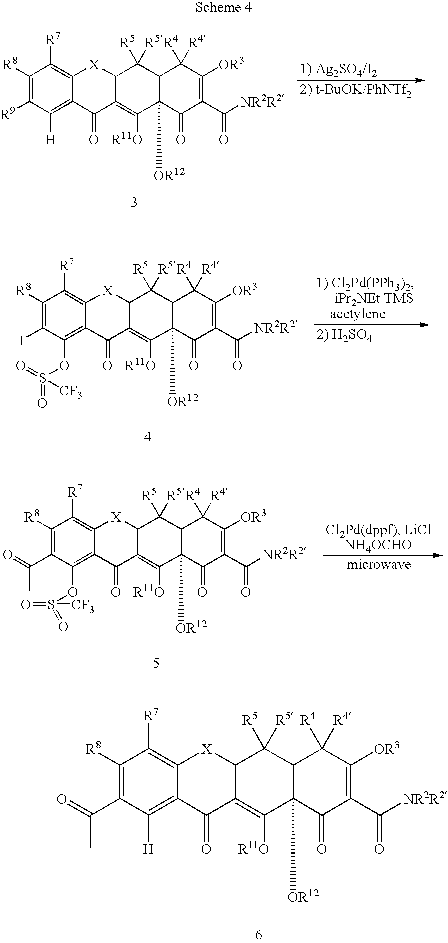

In one embodiment, the 10-substituted tetracycline can be prepared via conversion of a variety of anhydrous tetracycline freebases to the C9 iodo substituted tetracycline, followed by reaction of the C10 phenol with a base and a sulfonating agent to form a 9-iodo-10-sulfonyl-substituted tetracycline compound (Scheme 4, 4). Subsequently, the C9 iodo-substituted tetracycline can be converted to a C9 acyl substituted tetracycline compounds (5) by the use of nucleophilic acylation (COT, p. 718). Further reaction of the sulfonated C10 position by a reducing agent can provide the 9- and 10-substituted tetracycline compound (6).

For example, a 9-aminotetracycline can be converted to the 9-amino-10-substituted tetracycline as seen in Scheme 5. The 9-aminotetracycline compound (7) is first subjected to a base followed by a sulfonating agent, to provide the 9-amino-10-sulfonated tetracycline (8), which is then reacted with a reducing agent to give the 9-amino-10-substituted tetracycline compound (10).

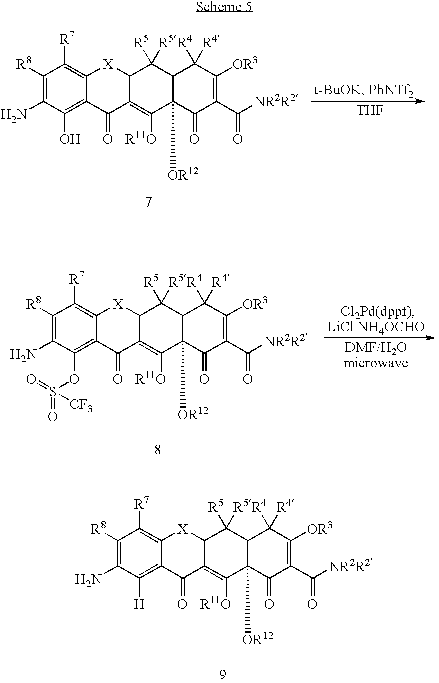

As seen in Scheme 6, a 9-iodo substituted tetracycline compound can also be converted to a 9-amino-10-substituted tetracycline compound by reaction of a 9-iodo-substituted tetracycline (10) first with a base and a sulfonating agent, followed by a nucleophilic acylation, then subjection of the 9-acyl-10-sulfonated tetracycline compound to a reducing agent. Finally, the 9-acyl-10-substituted tetracycline compound can be reacted nucleophile to provide the 9-amino-10-substituted tetracycline (9).

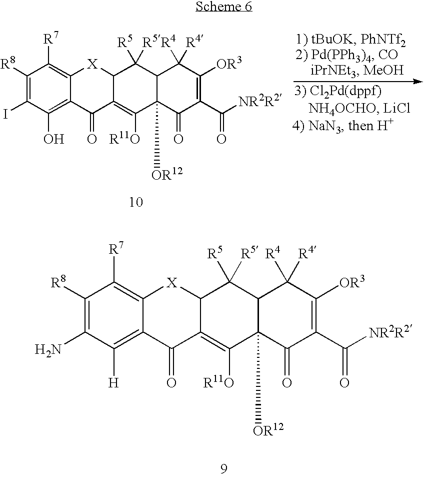

As seen in Scheme 7, a 10-substituted-4-dedimethylaamino tetracycline compound can be synthesized by converting a 4-dimethylamino substituted tetracycline freebase compound (11) to the 4-dedimethylamino tetracycline (12) using a reducing agent. Upon removal of the 4-position dimethylamino moiety, the resulting tetracycline can be exposed to a strong base and a sulfonating agent to provide a 4-dedimethylamino-10-sulfonated tetracycline (13), which can then be reacted with a reducing agent to give the 4-dedimethylamino-10-substituted tetracycline compound (14).

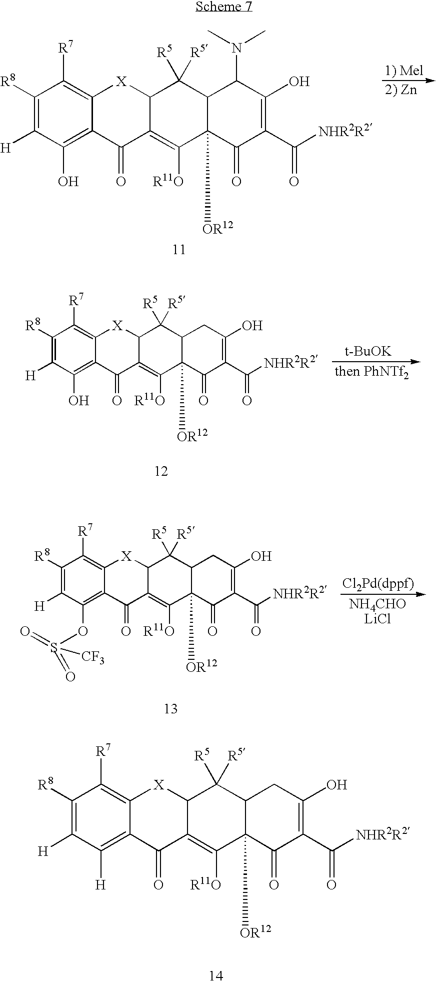

As seen in Scheme 8, a 10-amino substituted tetracycline compound (15) can be synthesized from 10-sulfonated tetracycline compound (2) by utilizing a reducing agent in the presence of an amine moiety. In Scheme 8, R^{30a} and R^{30b} are each independently hydrogen, alkyl, heterocyclic, aryl, alkenyl, alkynyl, alkoxy, carbonyl, acyl, halogen, cyano, amino, amid, hydroxy, alkoxy, or any other substituent described herein which would allow the tetracycline compounds of the invention to perform their intended functions. Other methods of converting a triflate to an amine group are also shown in Example 11. 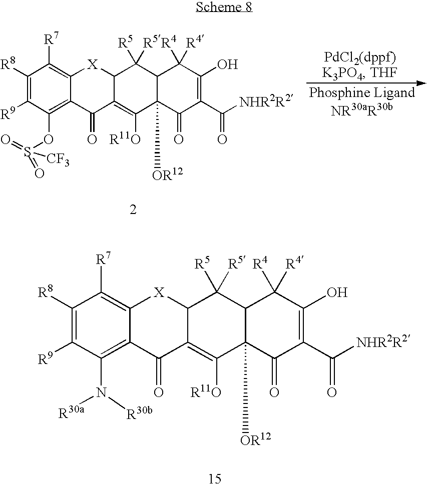
3. Methods for Using 10-Substituted Tetracycline Compounds

The invention also pertains to methods for treating a tetracycline responsive states in subjects, by administering to a subject an effective amount of a tetracycline compound of the invention (e.g., a compound of Formula I, II, III, IV, V or otherwise described herein), such that the tetracycline responsive state is treated.

The term “treating” includes curing as well as ameliorating at least one symptom of the state, disease or disorder, e.g., the tetracycline compound responsive state.

The language “tetracycline compound responsive state” or “tetracycline responsive state” includes states which can be treated, prevented, or otherwise ameliorated by the administration of a tetracycline compound of the invention, e.g., a tetracycline compound of Formula I, II, III, IV or V,or otherwise described herein). Tetracycline compound responsive states include bacterial, viral, and fungal infections (including those which are resistant to other tetracycline compounds), cancer (e.g., prostate, breast, colon, lung melanoma and lymph cancers and other disorders characterized by unwanted cellular proliferation, including, but not limited to, those described in U.S. Pat. No. 6,100,248), arthritis, osteoporosis, diabetes, and other states for which tetracycline compounds have been found to be active (see, for example, U.S. Pat. Nos. 5,789,395; 5,834,450; 6,277,061 and 5,532,227, each of which is expressly incorporated herein by reference). Compounds of the invention can be used to prevent or control important mammalian and veterinary diseases such as diarrhea, urinary tract infections, infections of skin and skin structure, ear, nose and throat infections, wound infection, mastitis and the like. In addition, methods for treating neoplasms using tetracycline compounds of the invention are also included (van der Bozert et al., Cancer Res., 48:6686-6690 (1988)). In a further embodiment, the tetracycline responsive state is not a bacterial infection. In another embodiment, the tetracycline compounds of the invention are essentially non-antibacterial. For example, non-antibacterial tetracycline compounds of the invention may have MIC values greater than about 4 μg/ml (as measured by assays known in the art and/or the assay given in Example 11).

Tetracycline compound responsive states also include inflammatory process associated states (IPAS). The term “inflammatory process associated state” includes states in which inflammation or inflammatory factors (e.g., matrix metalloproteinases (MMPs), nitric oxide (NO), TNF, interleukins, plasma proteins, cellular defense systems, cytokines, lipid metabolites, proteases, toxic radicals, adhesion molecules, etc.) are involved or are present in an area in aberrant amounts, e.g., in amounts which may be advantageous to alter, e.g., to benefit the subject. The inflammatory process is the response of living tissue to damage. The cause of inflammation may be due to physical damage, chemical substances, micro-organisms, tissue necrosis, cancer or other agents. Acute inflammation is short-lasting, lasting only a few days. If it is longer lasting however, then it may be referred to as chronic inflammation.

IPAS's include inflammatory disorders. Inflammatory disorders are generally characterized by heat, redness, swelling, pain and loss of function. Examples of causes of inflammatory disorders include, but are not limited to, microbial infections (e.g., bacterial and fungal infections), physical agents (e.g., burns, radiation, and trauma), chemical agents (e.g., toxins and caustic substances), tissue necrosis and various types of immunologic reactions.

Examples of inflammatory disorders include, but are not limited to, osteoarthritis, rheumatoid arthritis, acute and chronic infections (bacterial and fungal, including diphtheria and pertussis); acute and chronic bronchitis, sinusitis, and upper respiratory infections, including the common cold; acute and chronic gastroenteritis and colitis; acute and chronic cystitis and urethritis; acute and chronic dermatitis; acute and chronic conjunctivitis; acute and chronic serositis (pericarditis, peritonitis, synovitis, pleuritis and tendinitis); uremic pericarditis; acute and chronic cholecystis; acute and chronic vaginitis; acute and chronic uveitis; drug reactions; insect bites; burns (thermal, chemical, and electrical); and sunburn.

Tetracycline compound responsive states also include NO associated states. The term “NO associated state” includes states which involve or are associated with nitric oxide (NO) or inducible nitric oxide synthase (iNOS). NO associated state includes states which are characterized by aberrant amounts of NO and/or iNOS. Preferably, the NO associated state can be treated by administering tetracycline compounds of the invention, e.g., a compound of formula I, II, or otherwise described herein. The disorders, diseases and states described in U.S. Pat. Nos. 6,231,894; 6,015,804; 5,919,774; and 5,789,395 are also included as NO associated states. The entire contents of each of these patents are hereby incorporated herein by reference.

Other examples of NO associated states include, but are not limited to, malaria, senescence, diabetes, vascular stroke, neurodegenerative disorders (Alzheimer's disease & Huntington's disease), cardiac disease (reperfusion-associated injury following infarction), juvenile diabetes, inflammatory disorders, osteoarthritis, rheumatoid arthritis, acute, recurrent and chronic infections (bacterial, viral and fungal); acute and chronic bronchitis, sinusitis, and respiratory infections, including the common cold; acute and chronic gastroenteritis and colitis; acute and chronic cystitis and urethritis; acute and chronic dermatitis; acute and chronic conjunctivitis; acute and chronic serositis (pericarditis, peritonitis, synovitis, pleuritis and tendonitis); uremic pericarditis; acute and chronic cholecystis; cystic fibrosis, acute and chronic vaginitis; acute and chronic uveitis; drug reactions; insect bites; burns (thermal, chemical, and electrical); and sunburn.

The term “inflammatory process associated state” also includes, in one embodiment, matrix metalloproteinase associated states (MMPAS). MMPAS include states characterized by aberrant amounts of MMPs or MMP activity. These are also include as tetracycline compound responsive states which may be treated using compounds of the invention, e.g., a tetracycline compound of formula I, II, III, IV, V or otherwise described herein.

Examples of matrix metalloproteinase associated states (“MMPAS's”) include, but are not limited to, arteriosclerosis, corneal ulceration, emphysema, osteoarthritis, multiple sclerosis (Liedtke et al., Ann. Neurol. 1998, 44:35-46; Chandler et al., J. Neuroimmunol. 1997, 72:155-71), osteosarcoma, osteomyelitis, bronchiectasis, chronic pulmonary obstructive disease, skin and eye diseases, periodontitis, osteoporosis, rheumatoid arthritis, ulcerative colitis, inflammatory disorders, tumor growth and invasion (Stetler-Stevenson et al., Annu. Rev. Cell Biol. 1993, 9:541-73; Tryggvason et al., Biochim. Biophys. Acta 1987, 907:191-217; Li et al., Mol. Carcinog. 1998, 22:84-89)),metastasis, acute lung injury, stroke, ischemia, diabetes, aortic or vascular aneurysms, skin tissue wounds, dry eye, bone and cartilage degradation (Greenwald et al., Bone 1998, 22:33-38; Ryan et al, Curr. Op. Rheumatol. 1996, 8;238-247). Other MMPAS include those described in U.S. Pat. Nos. 5,459,135; 5,321,017; 5,308,839; 5,258,371; 4,935,412; 4,704,383, 4,666,897, and RE 34,656, incorporated herein by reference in their entirety.

In another embodiment, the tetracycline compound responsive state is cancer. Examples of cancers which the tetracycline compounds of the invention may be useful to treat include all solid tumors, i.e., carcinomas e.g., adenocarcinomas, and sarcomas. Adenocarcinomas are carcinomas derived from glandular tissue or in which the tumor cells form recognizable glandular structures. Sarcomas broadly include tumors whose cells are embedded in a fibrillar or homogeneous substance like embryonic connective tissue. Examples of carcinomas which may be treated using the methods of the invention include, but are not limited to, carcinomas of the prostate, breast, ovary, testis, lung, colon, and breast. The methods of the invention are not limited to the treatment of these tumor types, but extend to any solid tumor derived from any organ system. Examples of treatable cancers include, but are not limited to, colon cancer, bladder cancer, breast cancer, melanoma, ovarian carcinoma, prostatic carcinoma, lung cancer, and a variety of other cancers as well. The methods of the invention also cause the inhibition of cancer growth in adenocarcinomas, such as, for example, those of the prostate, breast, kidney, ovary, testes, and colon.

In an embodiment, the tetracycline responsive state of the invention is cancer. The invention pertains to a method for treating a subject suffering or at risk of suffering from cancer, by administering an effective amount of a substituted tetracycline compound, such that inhibition cancer cell growth occurs, i.e., cellular proliferation, invasiveness, metastasis, or tumor incidence is decreased, slowed, or stopped. The inhibition may result from inhibition of an inflammatory process, down-regulation of an inflammatory process, some other mechanism, or a combination of mechanisms. Alternatively, the tetracycline compounds may be useful for preventing cancer recurrence, for example, to treat residual cancer following surgical resection or radiation therapy. The tetracycline compounds useful according to the invention are especially advantageous as they are substantially non-toxic compared to other cancer treatments. In a further embodiment, the compounds of the invention are administered in combination with standard cancer therapy, such as, but not limited to, chemotherapy.

Examples of tetracycline responsive states also include neurological disorders which include both neuropsychiatric and neurodegenerative disorders, but are not limited to, such as Alzheimer's disease, dementias related to Alzheimer's disease (such as Pick's disease), Parkinson's and other Lewy diffuse body diseases, senile dementia, Huntington's disease, Gilles de la Tourette's syndrome, multiple sclerosis (e.g., including but not limited to, relapsing and remitting multiple sclerosis, primary progressive multiple sclerosis, and secondary progressive multiple sclerosis), amylotrophic lateral sclerosis (ALS), progressive supranuclear palsy, epilepsy, and Creutzfeldt-Jakob disease; autonomic function disorders such as hypertension and sleep disorders, and neuropsychiatric disorders, such as depression, schizophrenia, schizoaffective disorder, Korsakoff's psychosis, mania, anxiety disorders, or phobic disorders; learning or memory disorders, e.g., amnesia or age-related memory loss, attention deficit disorder, dysthymic disorder, major depressive disorder, mania, obsessive-compulsive disorder, psychoactive substance use disorders, anxiety, phobias, panic disorder, as well as bipolar affective disorder, e.g., severe bipolar affective (mood) disorder (BP-1), bipolar affective neurological disorders, e.g., migraine and obesity. Further neurological disorders include, for example, those listed in the American Psychiatric Association's Diagnostic and Statistical manual of Mental Disorders (DSM), the most current version of which is incorporated herein by reference in its entirety.

The language “in combination with” another therapeutic agent or treatment includes co-administration of the tetracycline compound with the other therapeutic agent or treatment, administration of the tetracycline compound first, followed by the other therapeutic agent or treatment and administration of the other therapeutic agent or treatment first, followed by the tetracycline compound. The other therapeutic agent may be any agent who is known in the art to treat, prevent, or reduce the symptoms of an IPAS or other tetracycline compound responsive state. Furthermore, the other therapeutic agent may be any agent of benefit to the patient when administered in combination with the administration of an tetracycline compound. In one embodiment, the cancers treated by methods of the invention include those described in U.S. Pat. Nos. 6,100,248; 5,843,925; 5,837,696; or 5,668,122, incorporated herein by reference in their entirety.

In another embodiment, the tetracycline compound responsive state is diabetes, e.g., juvenile diabetes, diabetes mellitus, diabetes type I, or diabetes type II. In a further embodiment, protein glycosylation is not affected by the administration of the tetracycline compounds of the invention. In another embodiment, the tetracycline compound of the invention is administered in combination with standard diabetic therapies, such as, but not limited to insulin therapy. In a further embodiment, the IPAS includes disorders described in U.S. Pat. Nos. 5,929,055; and 5,532,227, incorporated herein by reference in their entirety.

In another embodiment, the tetracycline compound responsive state is a bone mass disorder. Bone mass disorders include disorders where a subjects bones are disorders and states where the formation, repair or remodeling of bone is advantageous. For examples bone mass disorders include osteoporosis (e.g., a decrease in bone strength and density), bone fractures, bone formation associated with surgical procedures (e.g., facial reconstruction), osteogenesis imperfecta (brittle bone disease), hypophosphatasia, Paget's disease, fibrous dysplasia, osteopetrosis, myeloma bone disease, and the depletion of calcium in bone, such as that which is related to primary hyperparathyroidism. Bone mass disorders include all states in which the formation, repair or remodeling of bone is advantageous to the subject as well as all other disorders associated with the bones or skeletal system of a subject which can be treated with the tetracycline compounds of the invention. In a further embodiment, the bone mass disorders include those described in U.S. Pat. Nos. 5,459,135; 5,231,017; 5,998,390; 5,770,588; RE 34,656; 5,308,839; 4,925,833; 3,304,227; and 4,666,897, each of which is hereby incorporated herein by reference in its entirety.

In another embodiment, the tetracycline compound responsive state is acute lung injury. Acute lung injuries include adult respiratory distress syndrome (ARDS), post-pump syndrome (PPS), and trauma. Trauma includes any injury to living tissue caused by an extrinsic agent or event. Examples of trauma include, but are not limited to, crush injuries, contact with a hard surface, or cutting or other damage to the lungs.

The invention also pertains to a method for treating acute lung injury by administering a substituted tetracycline compound of the invention.

The tetracycline responsive states of the invention also include chronic lung disorders. The invention pertains to methods for treating chronic lung disorders by administering a tetracycline compound, such as those described herein. The method includes administering to a subject an effective amount of a substituted tetracycline compound such that the chronic lung disorder is treated. Examples of chronic lung disorders include, but are not limited, to asthma, cystic fibrosis, and emphysema. In a further embodiment, the tetracycline compounds of the invention used to treat acute and/or chronic lung disorders such as those described in U.S. Pat. No. 5,977,091; 6,043,231; 5,523,297; and 5,773,430, each of which is hereby incorporated herein by reference in its entirety.

In yet another embodiment, the tetracycline compound responsive state is ischemia, stroke, or ischemic stroke. The invention also pertains to a method for treating ischemia, stroke, or ischemic stroke by administering an effective amount of a substituted tetracycline compound of the invention. In a further embodiment, the tetracycline compounds of the invention are used to treat such disorders as described in U.S. Pat. No. 6,231,894; 5,773,430; 5,919,775 or 5,789,395, incorporated herein by reference.

In another embodiment, the tetracycline compound responsive state is a skin wound. The invention also pertains, at least in part, to a method for improving the healing response of the epithelialized tissue (e.g., skin, mucusae) to acute traumatic injury (e.g., cut, burn, scrape, etc.). The method may include using a tetracycline compound of the invention (which may or may not have antibacterial activity) to improve the capacity of the epithelialized tissue to heal acute wounds. The method may increase the rate of collagen accumulation of the healing tissue. The method may also decrease the proteolytic activity in the epthithelialized tissue by decreasing the collagenolytic and/or gellatinolytic activity of MMPs. In a further embodiment, the tetracycline compound of the invention is administered to the surface of the skin (e.g., topically). In a further embodiment, the tetracycline compound of the invention used to treat a skin wound, and other such disorders as described in, for example, U.S. Pat. Nos. 5,827,840; 4,704,383; 4,935,412; 5,258,371; 5,308,8391 5,459,135; 5,532,227; and 6,015,804; each of which is incorporated herein by reference in its entirety.

In yet another embodiment, the tetracycline compound responsive state is an aortic or vascular aneurysm in vascular tissue of a subject (e.g., a subject having or at risk of having an aortic or vascular aneurysm, etc.). The tetracycline compound may by effective to reduce the size of the vascular aneurysm or it may be administered to the subject prior to the onset of the vascular aneurysm such that the aneurysm is prevented. In one embodiment, the vascular tissue is an artery, e.g., the aorta, e.g., the abdominal aorta. In a further embodiment, the tetracycline compounds of the invention are used to treat disorders described in U.S. Pat. Nos. 6,043,225 and 5,834,449, incorporated herein by reference in their entirety.

Bacterial infections may be caused by a wide variety of gram positive and gram negative bacteria. The compounds of the invention are useful as antibiotics against organisms which are resistant to other tetracycline compounds. The antibiotic activity of the tetracycline compounds of the invention may be determined using the method discussed in Example 2, or by using the in vitro standard broth dilution method described in Waitz, J. A., National Commission for Clinical Laboratory Standards, Document M7-A2, vol. 10, no. 8, pp. 13-20, 2^{nd} edition, Villanova, Pa. (1990).

The tetracycline compounds may also be used to treat infections traditionally treated with tetracycline compounds such as, for example, rickettsiae; a number of gram-positive and gram-negative bacteria; and the agents responsible for lymphogranuloma venereum, inclusion conjunctivitis, psittacosis. The tetracycline compounds may be used to treat infections of, e.g., K. pneumoniae, Salmonella, E. hirae, A. baumanii, B. catarrhalis, H. influenzae, P. aeruginosa, E. faecium, E. coli, S. aureus or E. faecalis. In one embodiment, the tetracycline compound is used to treat a bacterial infection that is resistant to other tetracycline antibiotic compounds. The tetracycline compound of the invention may be administered with a pharmaceutically acceptable carrier. The tetracycline compounds of the invention may also be used to treat fungal disorders, viral disorders, parasitic disorders, and other disorders described in WO 03/005971, WO 02/085303, WO 02/072022, WO 02/072031, WO 01/52858, and U.S. Ser. No. 10/692764, each of which is incorporated herein by reference in its entirety.

In another embodiment, the tetracycline responsive state is a disorder treated by modulation of RNA.

The term “disorders treatable by modulation of RNA” or “DTMR” includes viral, neurodegenerative and other disorders which are caused or related to RNA function, structure, amounts and/or other activities of RNA which are lower or higher than desired and those disorders treatable by compounds described herein. Examples of DTMR include viral disorders (e.g., retroviral disorders (e.g., HIV, etc.), disorders caused by human rhinovirus RNA and proteins, VEE virus, Venezuelan equine encephalitis virus, eastern X disease, West Nile virus, bacterial spot of peach, camelpox virus, potato leafroll virus, stubborn disease and infectious variegations of citrus seedlings, viral protein synthesis in Escherichia coli infected with coliphage MS2, yellow viruses, citrus greening disease, ratoon stunting disease, European yellows of plants, inclusion conjunctivitis virus, meningopneumonitis virus, trachoma virus, hog plague virus, ornithosis virus, influenza virus, rabies virus, viral abortion in ungulates, pneumonitis, and cancer.

Other exemplary DTMRs include disorders caused by, or associated with splicing. For example, some disorders associated with defects in pre-mRNA processing result from a loss of function due to mutations in regulatory elements of a gene. Examples of such mutations are described in Krawczak et al. (1992) Hum. Genet, 90:41-54; and Nakai et al. (1994) Gene 14:171-177. Other DTMR include disorders which have been attributed to a change in trans-acting factors. Examples of DTMRs which are associated with splicing include those described in Philips et al. (2000), Cell. Mol. Life Sci., 57:235-249), as well as, FTDP-17 (frontotemporal dementia with parkinsonism) and β-thalassemia.

Certain DTMRs associated with splicing include those which are generated by point mutations that either destroy splice-sites or generate new cryptic sites in the vicinity of normally used exons. Examples of such DTMRs include cystic fibrosis (Friedman et al. (1999) J. Biol. Chem. 274:36193-36199), muscular dystrophy (Wilton et al. (1999) Neuromuscul. Disord. 9:330-338), and eosinophilic diseases (Karras et al., (2000) Mol. Pharamcol. 58:380-387).

Other DTMRs include cancers which may change splicing patterns during cancer formation and progression. Example of such cancers include, but are not limited to leukemia, colon/rectal cancer, myeloid leukemia, breast cancer, gastric carcinomas, acute leukemia, multiple myeloma, myeloid cell leukemia, lung cancer, prostate cancer, etc. Addition DTMRs associated with splicing are discussed in Stoss et al., (2000), Gene Ther. Mol. Biol. 5:9-30).

Another example of a DTMR is a cancer in which treatment of the cancer cells with a tetracycline compound results in the modulation of RNA, where the modulation of RNA increases the susceptibility of the cell to a second agent, e.g., a chemotherapeutic agent. Such DTMRs can be treated using a combination of the tetracycline compound and a chemotherapeutic agent. Exemplary cancers include those in which the tetracycline compound modulates the form of BCL expressed by the cells.

Other DTMRs include disorders wherein particular ribozymes are present in aberrant quantities. Examples include breast cancer, hepatitis C virus (HCV), liver cirrhosis, and heptacellular carcinoma.

The language “effective amount” of the compound is that amount necessary or sufficient to treat or prevent a tetracycline compound responsive state. The effective amount can vary depending on such factors as the size and weight of the subject, the type of illness, or the particular tetracycline compound. For example, the choice of the tetracycline compound can affect what constitutes an “effective amount”. One of ordinary skill in the art would be able to study the aforementioned factors and make the determination regarding the effective amount of the tetracycline compound without undue experimentation.

The invention also pertains to methods of treatment against microorganism infections and associated diseases. The methods include administration of an effective amount of one or more tetracycline compounds to a subject. The subject can be either a plant or, advantageously, an animal, e.g., a mammal, e.g., a human.

In the therapeutic methods of the invention, one or more tetracycline compounds of the invention may be administered alone to a subject, or more typically a compound of the invention will be administered as part of a pharmaceutical composition in mixture with conventional excipient, i.e., pharmaceutically acceptable organic or inorganic carrier substances suitable for parenteral, oral or other desired administration and which do not deleteriously react with the active compounds and are not deleterious to the recipient thereof.

The invention also pertains to pharmaceutical compositions comprising a therapeutically effective amount of a tetracycline compound (e.g., a tetracycline compound of the formula I, II, III, IV, V or otherwise described herein) and, optionally, a pharmaceutically acceptable carrier.

The language “pharmaceutically acceptable carrier” includes substances capable of being coadministered with the tetracycline compound(s), and which allow both to perform their intended function, e.g., treat or prevent a tetracycline responsive state. Suitable pharmaceutically acceptable carriers include but are not limited to water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethyl-cellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with the active compounds of the invention.

The tetracycline compounds of the invention that are basic in nature are capable of forming a wide variety of salts with various inorganic and organic acids. The acids that may be used to prepare pharmaceutically acceptable acid addition salts of the tetracycline compounds of the invention that are basic in nature are those that form non-toxic acid addition salts, i.e., salts containing pharmaceutically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and palmoate [i.e., 1,1′-methylene-bis-(2-hydroxy-3-naphthoate)]salts. Although such salts must be pharmaceutically acceptable for administration to a subject, e.g., a mammal, it is often desirable in practice to initially isolate a tetracycline compound of the invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The preparation of other tetracycline compounds of the invention not specifically described in the foregoing experimental section can be accomplished using combinations of the reactions described above that will be apparent to those skilled in the art.

The preparation of other tetracycline compounds of the invention not specifically described in the foregoing experimental section can be accomplished using combinations of the reactions described above that will be apparent to those skilled in the art.

The tetracycline compounds of the invention that are acidic in nature are capable of forming a wide variety of base salts. The chemical bases that may be used as reagents to prepare pharmaceutically acceptable base salts of those tetracycline compounds of the invention that are acidic in nature are those that form non-toxic base salts with such compounds. Such non-toxic base salts include, but are not limited to those derived from such pharmaceutically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines. The pharmaceutically acceptable base addition salts of tetracycline compounds of the invention that are acidic in nature may be formed with pharmaceutically acceptable cations by conventional methods. Thus, these salts may be readily prepared by treating the tetracycline compound of the invention with an aqueous solution of the desired pharmaceutically acceptable cation and evaporating the resulting solution to dryness, preferably under reduced pressure. Alternatively, a lower alkyl alcohol solution of the tetracycline compound of the invention may be mixed with an alkoxide of the desired metal and the solution subsequently evaporated to dryness.

The preparation of other tetracycline compounds of the invention not specifically described in the foregoing experimental section can be accomplished using combinations of the reactions described above that will be apparent to those skilled in the art.

The tetracycline compounds of the invention and pharmaceutically acceptable salts thereof can be administered via either the oral, parenteral or topical routes. In general, these compounds are most desirably administered in effective dosages, depending upon the weight and condition of the subject being treated and the particular route of administration chosen. Variations may occur depending upon the species of the subject being treated and its individual response to said medicament, as well as on the type of pharmaceutical formulation chosen and the time period and interval at which such administration is carried out.

The pharmaceutical compositions of the invention may be administered alone or in combination with other known compositions for treating tetracycline responsive states in a subject, e.g., a mammal. Preferred mammals include pets (e.g., cats, dogs, ferrets, etc.), farm animals (cows, sheep, pigs, horses, goats, etc.), lab animals (rats, mice, monkeys, etc.), and primates (chimpanzees, humans, gorillas). The language “in combination with” a known composition is intended to include simultaneous administration of the composition of the invention and the known composition, administration of the composition of the invention first, followed by the known composition and administration of the known composition first, followed by the composition of the invention. Any of the therapeutically composition known in the art for treating tetracycline responsive states can be used in the methods of the invention.

The tetracycline compounds of the invention may be administered alone or in combination with pharmaceutically acceptable carriers or diluents by any of the routes previously mentioned, and the administration may be carried out in single or multiple doses. For example, the novel therapeutic agents of this invention can be administered advantageously in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the therapeutically-effective compounds of this invention are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration (including intraperitoneal, subcutaneous, intravenous, intradermal or intramuscular injection), solutions of a therapeutic compound of the present invention in either sesame or peanut oil or in aqueous propylene glycol may be employed. The aqueous solutions should be suitably buffered (preferably pH greater than 8) if necessary and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art. For parenteral application, examples of suitable preparations include solutions, preferably oily or aqueous solutions as well as suspensions, emulsions, or implants, including suppositories. Therapeutic compounds may be formulated in sterile form in multiple or single dose formats such as being dispersed in a fluid carrier such as sterile physiological saline or 5% saline dextrose solutions commonly used with injectables.

Additionally, it is also possible to administer the compounds of the present invention topically when treating inflammatory conditions of the skin. Examples of methods of topical administration include transdermal, buccal or sublingual application. For topical applications, therapeutic compounds can be suitably admixed in a pharmacologically inert topical carrier such as a gel, an ointment, a lotion or a cream. Such topical carriers include water, glycerol, alcohol, propylene glycol, fatty alcohols, triglycerides, fatty acid esters, or mineral oils. Other possible topical carriers are liquid petrolatum, isopropylpalmitate, polyethylene glycol, ethanol 95%, polyoxyethylene monolauriate 5% in water, sodium lauryl sulfate 5% in water, and the like. In addition, materials such as anti-oxidants, humectants, viscosity stabilizers and the like also may be added if desired.

For enteral application, particularly suitable are tablets, dragees or capsules having talc and/or carbohydrate carrier binder or the like, the carrier preferably being lactose and/or corn starch and/or potato starch. A syrup, elixir or the like can be used wherein a sweetened vehicle is employed. Sustained release compositions can be formulated including those wherein the active component is derivatized with differentially degradable coatings, e.g., by microencapsulation, multiple coatings, etc.

In addition to treatment of human subjects, the therapeutic methods of the invention also will have significant veterinary applications, e.g. for treatment of livestock such as cattle, sheep, goats, cows, swine and the like; poultry such as chickens, ducks, geese, turkeys and the like; horses; and pets such as dogs and cats. Also, the compounds of the invention may be used to treat non-animal subjects, such as plants.

It will be appreciated that the actual preferred amounts of active compounds used in a given therapy will vary according to the specific compound being utilized, the particular compositions formulated, the mode of application, the particular site of administration, etc. Optimal administration rates for a given protocol of administration can be readily ascertained by those skilled in the art using conventional dosage determination tests conducted with regard to the foregoing guidelines.

In general, compounds of the invention for treatment can be administered to a subject in dosages used in prior tetracycline therapies. See, for example, the Physicians' Desk Reference. For example, a suitable effective dose of one or more compounds of the invention will be in the range of from 0.01 to 100 milligrams per kilogram of body weight of recipient per day, preferably in the range of from 0.1 to 50 milligrams per kilogram body weight of recipient per day, more preferably in the range of 1 to 20 milligrams per kilogram body weight of recipient per day. The desired dose is suitably administered once daily, or several sub-doses, e.g. 2 to 5 sub-doses, are administered at appropriate intervals through the day, or other appropriate schedule.

It will also be understood that normal, conventionally known precautions will be taken regarding the administration of tetracyclines generally to ensure their efficacy under normal use circumstances. Especially when employed for therapeutic treatment of humans and animals in vivo, the practitioner should take all sensible precautions to avoid conventionally known contradictions and toxic effects. Thus, the conventionally recognized adverse reactions of gastrointestinal distress and inflammations, the renal toxicity, hypersensitivity reactions, changes in blood, and impairment of absorption through aluminum, calcium, and magnesium ions should be duly considered in the conventional manner.

Furthermore, the invention also pertains to the use of a tetracycline compound of formula I, II, III, IV or V or a compound otherwise described herein for the preparation of a medicament. The medicament may include a pharmaceutically acceptable carrier and the tetracycline compound is an effective amount, e.g., an effective amount to treat a tetracycline responsive state.

Exemplification of the Invention

Compounds of the invention may be made as described below and/or by using literature techniques known to those of ordinary skill of the art.

### EXAMPLE 1

### Preparation of Anhydrous Freebase Sancycline

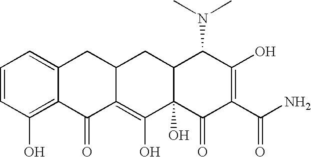

A solution of sancycline in a water solution of 1% TFA was slowly stirred for several hours until a suspension was obtained. The suspension was filtered through a 0.2 μm nylon membrane filter and rinsed with 1% TFA in water to collect a tan solid. The resulting solid was dissolved in 10% CH₃CN in water and loaded onto a DVB resin column. After the solution was loaded, a 1M solution of NaOAc was eluted until the eluent became basic, then distilled water was eluted to remove excess NaOAC until a neutral pH was obtained. The freebase sancycline was eluted with a solution of 1:1 CH₃CN:MeOH, and the yellow eluent was collected until the eluent became colorless. The solution was concentrated under reduced pressure and the sancycline was further dried by azetroping the water with anhydrous toluene using a Dean-Stark trap for approximately 1 hour. After cooling the solution to ambient temperature, the solution was concentrated under reduced pressure and high vacuum for 24 hours.

### EXAMPLE 2

### Preparation of Anydrous Freebase Minocycline

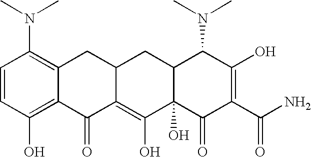

A dissolved solution of minocycline in a 1% TFA water solution was loaded onto a DVB resin column. After the solution was loaded, a 1M solution of NaOAc was eluted until the eluent became basic, then distilled water was eluted to remove excess NaOAC until a neutral pH was obtained. The freebase minocycline was eluted with a solution of 1:1 CH₃CN:MeOH, and the yellow eluent was collected until the eluent became colorless. The solution was concentrated under reduced pressure and the sancycline was further dried by azetroping the water with anhydrous toluene using a Dean-Stark trap for approximately 1 hour. After cooling the solution to ambient temperature, the solution was concentrated under reduced pressure and high vacuum for 24 hours.

### EXAMPLE 3

### Preparation of Sancycline-10-triflate

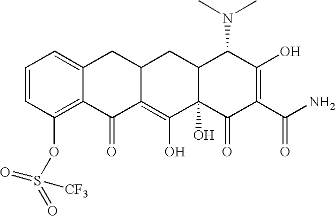

To a solution of anhydrous freebase sancycline (10.4 g, 25.0 mmol) in anhydrous THF (163 mL) under argon was added a 1 M solution of potassium t-butoxide (87.5 mL, 87.5 mmol) dropwise. After 45 minutes, solid N-phenylbis(trifluoromethane sulfonimide) (18.8 g, 52.5 mmol) was added at once. After 1 hour, the solution was allowed to slowly warm to room temperature. After another 2 hours, the solution was poured into cold 1 M HCl (1 L). The water solution was extracted twice with MTBE (750 mL). The water layer was loaded onto a column packed with DVB resin. After the solution was loaded, a 1 M solution of NaOAc was eluted until the eluent became basic, then distilled water was eluted to remove excess NaOAc until a neutral pH was obtained. The sancycline-10-triflate was eluted with a solution of 1:1 CH₃CN:EtOH and the yellow eluent was collected until the eluent became colorless. The solution was concentrated under reduce pressure and further dried under high vacuum to afford a light brown solid. Spectroscopic data: MS (MH+)=574; ¹H NMR (300 MHz, CD₃OD) δ 7.62 (m, 1 H), 7.40 (m, 1 H), 4.02 (s, 1 H), 3.11-2.89 (m, 9 H), 2.65 (m, 1 H), 2.19-2.10 (m, 1 H), 1.71-1.58 (m, 1 H).

### EXAMPLE 4

### Preparation of Minocycline-10-triflate

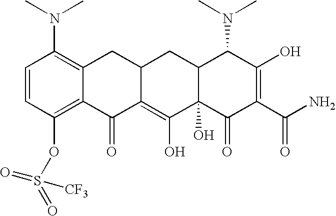

To a solution of anhydrous freebase sancycline (11.4 g, 25.0 mmol) in anhydrous THF (163 mL) under argon was added a 1 M solution of potassium t-butoxide (87.5 mL, 87.5 mmol) dropwise. After 45 minutes, solid N-phenylbis(trifluoromethane sulfonimide) (18.8 g, 52.5 mmol) was added at once. After 1 hour, the solution was allowed to slowly warm to room temperature. After another 2 hours, the solution was poured into cold 1 M HCl (1 L). The water solution was extracted twice with MTBE (750 mL). The water layer was loaded onto a column packed with DVB resin. After the solution was loaded, a 1 M solution of NaOAc was eluted until the eluent became basic, then distilled water was eluted to remove excess NaOAc until a neutral pH was obtained. The minocycline-10-triflate was eluted with a solution of 1:1 CH₃CN:EtOH and the yellow eluent was collected until the eluent became colorless. The solution was concentrated under reduce pressure and further dried under high vacuum to afford a light brown solid.

### EXAMPLE 5

### Preparation of 9-Iodominocycline-10-Triflate

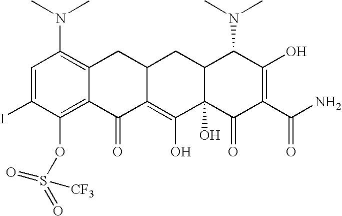

To a solution of anhydrous freebase 9-iodominocycline (12.3 g, 21.1 mmol) in anhydrous THF (211 mL) under argon at 0° C. was added solid potassium t-butoxide (7.10 g, 63.3 mmol). After 45 minutes, N-phenylbis(trifluoromethanesulfonimide) (15.8 g, 63.3 mmol) was added at once. After 1 hour, the solution was allowed to slowly warm to room temperature. After about 18 hours, the solution was slowly poured into a vigorously stirring solution of 0.1H HCl and Celite. After 15 minutes, the solution was filtered through a large plug of Celite while rinsing with 0.1 M HCl. The solution was loaded onto a column packed with DVB resin. After the solution was loaded, a 1 M solution of NaOAc was eluted until the eluent became basic, then distilled water was eluted to remove excess NaOAc until a neutral pH was obtained. The 9-iodominocycline-10-triflate was eluted with a solution of 1:1 CH₃CN:EtOH and the yellow eluent was collected until the eluent became colorless. The solution was concentrated under reduce pressure and further dried under high vacuum to afford a light brown solid.

### EXAMPLE 6

### Preparation of 10-Deoxytetracycline

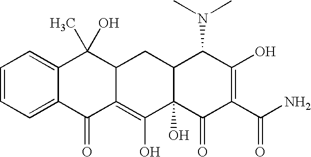

To a solution of tetracycline-10-triflate freebase (3.5 mmol) in DMF (10 mL) and water (10 mL) was added ammonium formate (0.66 g, 10.5 mmol), LiCl (0.30 g, 7.0 mmol) and Cl₂Pd(dppf) (0.022 g, 0.175 mmol) in a 20 mL Biotage microwave vial. The secured vial was placed into a Biotage microwave reaction with a temperature setting of 100° C. for 7 minutes. After cooling, the vial was opened and poured into a 1% TFA in water. The solution was filtered through a plug of Celite and rinsed with 1% TFA in water until the filtrate became colorless. The water solution was loaded onto a column packed with DVB resin. After the solution was loaded, distilled water was eluted to remove salts, then CH₃CN was eluted and the yellow eluent was collected until the eluent became colorless. The solution was concentrated under reduced pressure and further purified by preparatory chromatography. The combined fractions were concentrated under reduced pressure to afford a pale yellow solid.

### EXAMPLE 7

### Preparation of 4-Dedimethylaminominocycline

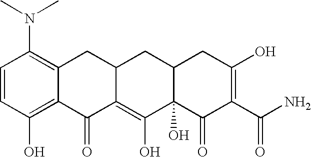

To a solution of anhydrous minocycline (30.5 g, 66.7 mmol) in anhydrous THF (250 mL) under argon was added iodomethane (41.5 mL, 667 mmol). The solution was heated to 45° C. for 24 hours. After cooling to room temperature, the solution was poured into a vigorously stirring solution of 1:1 hexanes/ether to precipitate the product. The suspension was collected on a fine sintered funnel, followed by rinsing with 1:1 hexanes/ether. The product, minocycline-4-methyl ammonium salt, was dried under high vacuum to yield 40 g of a light brown solid. To a solution of the anhydrous minocycline-4-methyl ammoniom salt (2.40 g, 4.00 mmol) in anhydrous NMP (20 mL) was added Zn powder (0.523 g, 8.00 mmol) and acetic acid (0.025 mL, 0.400 mmol) in a 20 mL microwave vial. The secured vial was placed into a microwave reactor with a setting or 120° C. for 25 minutes. After cooling, the vial was opened and the contents were poured into a 1% TFA/water solution and stirred for 20 minutes. The water solution was loaded onto a prepared DVB resin column. After the solution was loaded, distilled water was eluted, then CH₃CN was eluted and the yellow eluent was collected until the eluent became colorless. The solution was concentrated under reduced pressure and further purified using preparatory chromatography. The combined fractions were concentrated under reduced pressure to afford 1.2 g of the 4-dedimethylamino minocycline as a pale yellow solid in 71% yield.

### EXAMPLE 8

### Preparation of 4-dedimethylaminominocycline-10-triflate

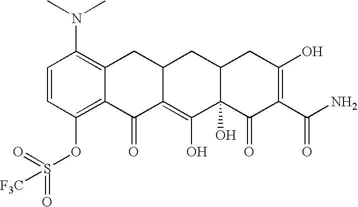

To a solution of anhydrous 4-dedimethylaminominocycline (10.4 g, 25.0 mmol) in anhydrous THF (163 mL) at 0° C. was added a 2.0 M solution of potassium tert-butoxide (87.5 mL, 87.5 mmol) dropwise. After 45 minutes, solid N-phenylbis(trifluoromethanesulfonimide) (18.8 g, 52.5 mmol) was added in one portion. After 2 hours, the solution was poured into 0.5 M HCl (1 L). The solution was extracted two times with MTBE (700 mL). The combined organic layers were dried over anhydrous Na₂SO₄, then concentrated under reduced pressure. The crude 4-dedimethylaminominocycline-10-triflate was used as is in the following reaction.

### EXAMPLE 9

### Preparation of 4-Dedimethylamino-10-Deoxyminocycline

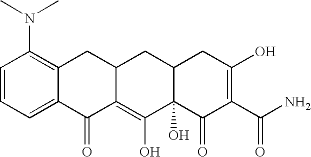

To a solution of 4-dedimethylaminominocycline-10-triflate (1.91 g, 3.50 mmol) in NMP (10 mL) and water (10 mL) was added ammonium formate (0.662 g, 10.5 mmol), LiCl (0.297 g, 7.00 mmol) and Cl₂Pd(dppf) (0.022 g, 0.175 mmol) in a 20 mL microwave vial. The secured vial was placed into a microwave reaction with a temperature setting of 100° C. for 15 minutes. After cooling, the contents of the vial was poured into a 1% TFA/water solution. The solution was filtered through a plug of Celite and rinsed with 1% TFA/water until the filtrate became colorless. The water solution was loaded onto a prepared DVB resin column. After the solution was loaded, distilled water was eluted, then CH₃CN was eluted and the yellow eluent was collected until the eluent became colorless. The solution was concentrated under reduced pressure and further purified by preparatory chromatography. The combined fractions were concentrated under reduced pressure to afford 0.95 g as a pale yellow solid in 65% yield. Spectroscopic data: MS (MH+)=399; ¹H NMR (300 MHz, CD₃OD) δ 8.10 (m, 1 H), 7.92 (m, 1 H), 7.62 (m, 1 H), 3.30-3.14 (m, 2 H), 3.04-2.95 (m, 1 H), 2.60-2.37 (m, 3 H), 2.18 (m, 1 H), 1.67 (m, 1 H).

### EXAMPLE 10

### Preparation of Sancycline-10-Amine

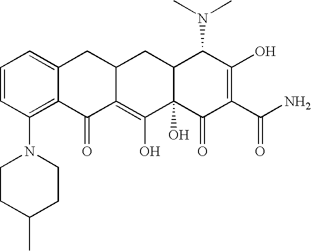

Sancycline-10-triflate (0.6 g, 1.09 mmol) was combined with solid anhydrous potassium phosphate (0.7 g,. 3.30 mmol), PdCl₂(dppf) (0.16 g, 0.218 mmol), 2-(di-ter-butylphosphino)biphenyl (0.65 g, 0.218 mmol) and anhydrous THF (10 mL) in a 20 mL microwave vial. An amount of 4-methyl piperidine (0.643 mL, 5.45 mmol) was added to the reaction mixture, the vial was sealed and reacted in a microwave reactor at 105° C. for 20 minutes., 110° C. for 50 minutes., then 120° C. for 15 minutes. The solvent was evaporated under reduced pressure. A 0.1% TFA/water solution (300 mL) was added to the dried reaction mixture and a heterogenous mixture resulted. The solution was filtered through a sintered glass funnel and the aqueous layer was loaded onto a prepared 5 g DVB cartridge. The product was washed with water (0.1% TFA), then 1:1 CH₃OH (0.1% TFA):H₂O(0.1% TFA). The product was eluted as a bright yellow band and was evaporated under reduced pressure. The product was purified in 2 batches on a 2″ C-18 Luna column using a 10-5% CH₃CN (0.1% TFA) gradient over 35 minutes. The resulting pure product was evaporated under reduced pressure and redissolved in saturated MeOH (HCl) to afford the HCl salt. This final product was dried overnight under high vacuum with P₂O₅ to yield 30 mg of a light yellow solid. Spectroscopic data: MS (MH+)=496; ¹H NMR (300 MHz, CD₃OD) δ 7.90-7.70 (m, 2 H), 7.58-7.47 (m, 1 H), 4.07 (s, 1 H), 3.85-3.51 (m, 4 H), 3.20-3.05 (m, 2 H), 3.01-2.80 (m, 8 H), 2.72-2.53 (m, 1 H), 2.30-1.49 (m, 8 H), 1.07 (d, J=6.0, 3 H).

### EXAMPLE 11

### Preparation 10-N,N-Dialkylaminosancycline

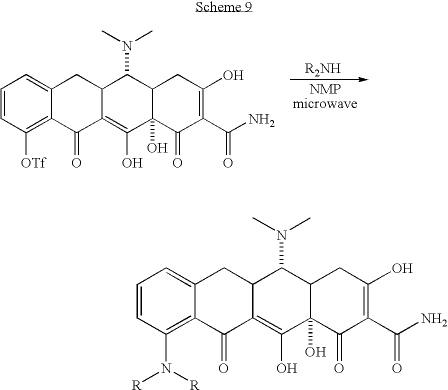

To a solution of sancycline-10 triflate (2.18 g, 4.00 mmol) in anhydrous NMP (20 mL) was added R₂NH (20.0 mmol) in a 20 mL Biotage microwave vial. The secured vial was placed into a Biotage microwave reactor with a temperature setting of 110° C. for 25 minutes. After cooling, the vial was opened and poured into a 1% TFA/water solution. The water solution was loaded onto a prepared DVB resin for semi-purification. After the solution was loaded, distilled water was eluted, and then CH₃CN with 1% TFA was eluted where the yellow eluent was collected until the eluent became colorless. The solution was concentrated under reduced pressure and further purified on preparatory chromatography. The combined fractions were concentrated under reduced pressure to afford a light brown solid. Compounds made by using this method include Compounds AF, AG, and AH.

### EXAMPLE 12

### Spectroscopic Data

The following compounds were synthesized using the methods described in Examples 1-11 and other techniques known in the art.

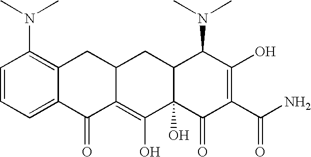

MS (MH+)=515; ¹H NMR (300 MHz, CD₃OD) δ 8.12 (m, 1 H), 7.93 (m, 1 H), 7.66 (m, 1 H), 4.83 (s, 1 H), 4.40-3.95 (m, 9 H), 2.58 (m, 1 H), 2.29 (m, 1 H), 1.70 (m, 1H).

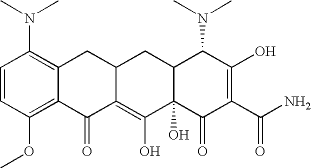

MS (MH+)=545; ¹H NMR (300 MHz, CD₃OD) δ 7.94 (d, J=7.8 Hz, 1 H), 7.31 (d, J=7.8 Hz, 1 H), 4.18 (s, 1 H), 4.00 (s, 3 H), 3.37-3.01 (m, 9 H), 2.51 (m, 1 H), 2.36 (m, 1 H), 1.56 (m, 1 H).

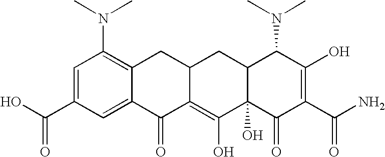

MS (MH+)=559; ¹H NMR (300 MHz, CD₃OD) δ 8.62 (d, J=2.0 Hz, 1 H), 8.40 (d, J=2.0 Hz, 1 H), 4.07 (s, 1 H), 3.44-2.92 (m, 9 H), 2.59 (m, 1 H), 2.33 (m, 1 H), 1.71 (m, 1 H).

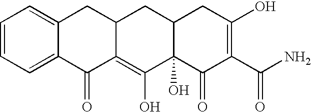

MS (MH+)=356; ¹H NMR (300 MHz, CD₃OD) δ 7.89 (m, 1 H), 7.47 (m, 1 H), 7.31 (m, 1 H), 7.18 (m, 1 H), 3.22 (m, 1 H), 2.90-2.72 (m, 2 H), 2.59-2.32 (m, 3 H), 2.00 (m, 1 H), 1.58 (m, 1 H).

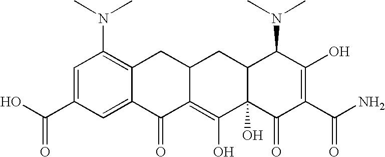

MS (MH+)=486; ¹H NMR (300 MHz, CD₃OD) δ 7.57 (s, 1 H), 7.48 (s, 1 H), 4.38 (s, 1 H), 3.25-2.92 (m, 9 H), 2.58 (m, 1 H), 2.22 (m, 1 H), 1.67 (m, 1 H).

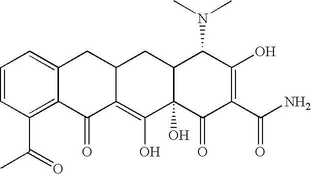

MS (MH+)=441; ¹H NMR (300 MHz, CD₃OD) δ 7.52 (t, J=7.6 Hz, 1 H), 7.33 (d, J=7.6 Hz, 1 H), 7.17 (d, J=7.6 Hz, 1 H), 4.05-3.95 (m, 1 H), 3.20-2.82 (m, 11 H), 2.62-2.45 (m, 1 H), 2.45 (s, 3 H), 2.20-2.10 (m, 1 H) 2.65-2.45 (m, 1H), 1.30-1.20 (m, 1 H).

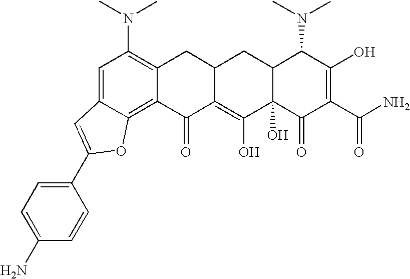

MS (MH+)=573; ¹H NMR (300 MHz, CD₃OD) δ 8.35-8.25 (m, 1 H), 8.02-7.90 (m, 1 H), 7.65-7.55 (m, 1 H), 7.29 (s, 1 H), 4.25 (s, 0.9 H), 3.60-3.40 (m, 6 H), 3.40-3.30 (m, 4 H), 3.30-3.00 (m, 8 H) 2.70-2.55 (m, 1H), 2.51-2.30 (m, 1 H), 1.90-1.70 (m, 1 H).

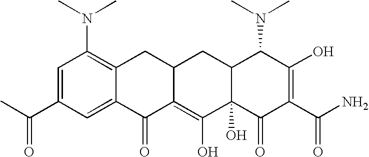

MS (MH+)=484; ¹H NMR (300 MHz, CD₃OD): Product forms a hemi-acetal with deuterated methanol, δ 8.62 and 8.25 (from hemi-acetal, 1 H), 8.38 and 8.03 (from hemi-acetal, 1 H), 4.15 (s, 1 H), 3.42-3.38 (m, 1 H), 3.30-3.10 (m, 2 H), 3.10-3.00 (m, 12 H), 2.72 and 1.59 (from hemi-acetal, 3 H), 2.68-2.56 (m, 1 H), 2.38 (m, 1H), 1.76 (m, 1H).

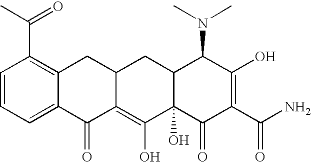

LCMS, MH+=441; ¹H NMR (300 MHz, CD₃OD) δ 8.14 (1H, dd, J=7.8 Hz, 1.0 Hz), 7.98 (1H, dd, J=7.8 Hz, 1.1 Hz), 7.52 (1H, t, J=7.8 Hz), 4.84 (1H, d, J=3.8 Hz), 3.23-2.97 (8H), 2.61 (s, 1H), 2.51 (m, 1H), 2.10 (m, 1H), 1.66 (m, 1H).

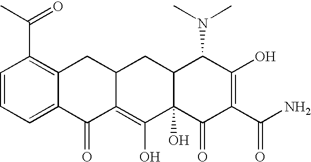

LCMS, MH+=441; ¹H NMR (300 MHz, CD₃OD) δ 8.15 (1H, dd, J=7.8 Hz, 1.2 Hz), 7.99 (1H, dd, J=7.8 Hz, 1.3 Hz), 7.53 (1H, t, J=7.8 Hz), 4.09 (s, 1H), 3.23-2.94 (8H), 2.62 (s, 1H), 2.50 (m, 1H), 2.22 (m, 1H), 1.67 (m, 1H).

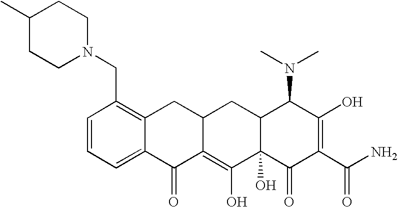

LCMS, MH+=510; ¹H NMR (300 MHz, CD₃OD) δ 8.12 (m, 1H), 7.77 (m, 1H), 7.53 (m, 1H), 4.85 (1H, d, J=4.0 Hz), 4.45 (m, 2H), 3.55-3.56 (3H), 3.24-3.04 (10H), 2.56-2.30 (2H), 1.93-1.52 (6H), 1.00 (3H, d, J=5.5 Hz).

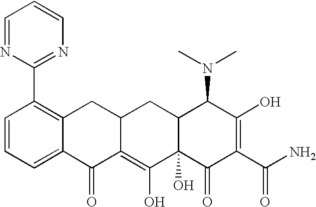

LCMS, MH+=477; ¹H NMR (300 MHz, CD₃OD) δ 8.90 (1H, t, J=4.4 Hz), 8.17 (s, 1H), 8.11 (1H, d, J=6.7 Hz), 7.90 (1H, d, J=7.5 Hz), 7.55-7.45 (2H), 4.68 (1H, d, J=3.8 Hz), 3.18-2.78 (9H), 2.61 (s, 1H), 2.62 (m, 1H), 2.01 (m, 1H), 1.61 (m, 1H).

### EXAMPLE 13

### In Vitro Minimum Inhibitory Concentration (MIC) Assay for S. aureus, S. pneumoniae, E. coli, P. aeruginosa and H. influenza

The following assay was used to determine the efficacy of tetracycline compounds against common bacteria. Serial dilutions of compounds were prepared in microdilution plates using a Tecan robotic workstation. Mueller Hinton broth cultures of gram negative and gram positive strains were grown or adjusted to match the turbidity of a 0.5 McFarland standard. 1:200 dilutions were made in appropriate broth (cation supplemented Mueller Hinton broth or Haemophilus test medium) to allow a final inoculum of 1×10⁵ cfu. Lysed horse blood was used to supplement broth for testing S. pneumoniae. The plates were incubated at 35° C. in ambient air for 18-24 hours, read spectrophotometrically, and checked manually for evidence of bacterial growth. Serial dilutions of compounds were also added to Brucella agar supplemented with laked sheep blood for P. acnes tests. A standard inoculum was transferred to the plate using a stainless steel replicator. After 48 hours of anaerobic incubation at 35° C. plates were examined. The MIC was defined as the lowest concentration of the tetracycline compound that inhibits growth. The results of this assay are given in Table 3. Table 3 gives the MIC (μg/mL) of selected substituted tetracycline compounds against a variety of gram positive and gram negative bacteria. Compounds which showed superior inhibition of these bacteria are indicated by “ * * *,” (0-21 μg/mL) and compounds which showed very good or good inhibition of bacteria are indicated by “**” (22-43 μg/mL) or “*” (44-64 μg/mL) respectively. The designation “ND” indicates that no value was obtained.

TABLE 3

| | S. aureus | S. aureus | S. aureus | S. pneumoniae | E. coli | E. coli MG 1655 | P. aeruginosa | H. influenza |
|---|---|---|---|---|---|---|---|---|
| Code | RN450 | MRSA5 | RN4250 | 157E | (ATCC 25922) | DWAK | K1033 | (ATCC 49247) |
| A | *** | * | *** | *** | ** | *** | *** | ** |
| C | *** | * | *** | *** | * | *** | *** | ** |
| D | * | * | * | * | * | * | * | * |
| E | *** | *** | *** | * | * | *** | *** | ** |
| F | *** | *** | *** | ** | * | *** | *** | *** |
| G | * | * | * | * | * | * | * | * |
| H | * | * | * | * | * | ** | ** | * |
| I | *** | *** | *** | *** | ** | *** | *** | *** |
| J | *** | *** | *** | *** | *** | *** | *** | *** |
| K | *** | *** | *** | *** | *** | *** | *** | *** |
| L | *** | *** | *** | *** | ** | *** | *** | * |
| M | * | * | * | ** | * | *** | *** | *** |
| N | ** | * | * | * | * | *** | *** | * |
| O | ** | ** | ** | * | * | *** | *** | * |
| P | * | * | * | * | ND | ND | ND | * |
| Q | *** | *** | *** | ** | ND | ND | ND | * |

### EXAMPLE 14

### In Vitro Minimum Inhibitory Concentration (MIC) Assay for B. fragilis B. thetaiotaomicron, P. acnes and P. granulosum

The following data was obtained by following the experimental procedures outlined in Example 12 and the results are given in Table 4. Table 4 gives the MIC (μg/mL) of selected substituted tetracycline compounds against B. fragilis (ATCC 25285), B. theiaiotaomicron (ATCC 29741), P. acnes (ATCC 6919), P. acnes (11827), P. acnes (PBS 1073), P. acnes (PBS 1074), P. acnes (PBS 1077), P. acnes (PBS 1080), P. acnes (PBS 994), P. granulosum (PBS 1048) and P. granulosum (1098). Compounds which showed superior inhibition of are indicated by “ * * * ” (0-8 μg/mL), and compounds which showed very good or good inhibition are indicated by “**” (9-27 μg/mL) or “*” (28-32 μg/mL) respectively. The designation “ND” indicates that no value was obtained.

TABLE 4

| | B. fragilis | B. theraiotaomicron | P. acnes | P. acnes | P. acnes |
|---|---|---|---|---|---|
| Code | (ATCC 25285) | (ATCC 29741) | (ATCC 6919) | (ATCC 11827) | (PBS 1073) |
| A | *** | ** | *** | *** | ** |
| I | *** | * | *** | *** | * |
| J | *** | * | *** | *** | * |
| K | *** | * | *** | *** | * |
| L | *** | * | *** | *** | * |

| | P. acnes | P. acnes | P. acnes | P. acnes | P. granulosum | P. granulosum |
|---|---|---|---|---|---|---|
| Code | (PBS 1074) | (ATCC 1077) | (PBS 1080) | (PBS 994) | (PBS 1048) | (PBS 1098) |
| A | ** | ** | ** | ** | ** | ** |
| I | * | * | ND | ND | ND | ND |
| J | * | * | ND | ND | ND | ND |
| K | * | * | ND | ND | ND | ND |
| L | * | * | ND | ND | ND | ND |

### EXAMPLE 15

### In vitro Translation Assay for Inhibition of Bacterial Protein Synthesis

As a further measure of antibacterial activity, an E. coli S30 Extract System was used to quantitate inhibition of E. coli translation by tetracycline derivatives. This system contained all required components for translation, including a nucleic acid template which, when translated, produces functional luciferase. The reactions were set up with E. coli S30 extract, amino acids, DNA template and selected tetracycline compounds at concentrations of 100, 50, 25, 10, 5, and 1 μg/ml. The reactions were incubated at 37° C. for 1 hour and analyzed by adding the reaction mix to Steady-Glo Luciferase Assay substrate (Promega, Madison Wis.). Luminescence was measured with the Wallac Victor5 plate reader and inhibition is calculated by comparing luciferase activity in experimental reactions versus controls. The results of this assay are given in Table 6. Compounds which showed good or some inhibition are indicated by “**” (>100 μM) or “*” (<100 μM) respectively.

TABLE 6

| Code | IC₅₀ (μM) | IC₂₀ (μM) |
|---|---|---|
| A | ** | ** |
| A | ** | ** |
| A | ** | ** |
| C | ** | ** |
| D | * | * |
| E | * | ** |
| F | * | * |
| G | * | * |
| H | * | * |
| M | * | * |
| N | * | * |
| O | * | * |
| Q | * | * |

### EXAMPLE 16

### Evaluation of Efficacy in a Rat Model of Carrageenan Induced Paw Edema

Carrageenan-induced paw edema represents a commonly used experimental model to assess the anti-inflammatory properties of agents. In this example, male Sprague-Dawley rats (Charles River, Mass.) weighing 175-250 grams were used. Test compounds were administered intraperitoneally 5 minutes or orally 15 minutes before a subplantar injection of carrageenan (5 mg/1 ml) in the rat right hind paw. The paw volume (ml) was monitored with a plethysmometer (Water Plethysmometer) at time of carrageenan injection (baseline) and 3 hours after carrageenan injection. When dosed at 75 mg/kg IP, it was found that compound A caused a 60% decrease in paw inflammation relative to the untreated control. This result was comparable to the positive control, Minocycline (dosed at 50 mg/kg IP), which reduced inflammation 70% relative to the untreated control. Both results were statistically significant with a p<0.1 level by Kruskal-Wallis One Way ANOVA, ChiSquare. There were approximately four rats per group.

### EXAMPLE 17

### Mammalian Cell Cytotoxicity Assay and Phototoxicity

Cytotoxicity Assay

To predict the in vivo risks associated with the compounds of the invention, a soluble, non-toxic redox dye (“Resazurin”; Alamar Blue) was used to assess a compound's effect on cellular metabolism. COS-1 or CHO cell suspensions were prepared, seeded into 96-well black-walled microtiter plates, and incubated overnight at 37° C., in 5% CO₂ and approximately 95% humidity. On the next day, serial dilutions of test drug were prepared and transferred to cell plates. Following a 24 hour incubation period, the media/drug was aspirated, and 50 μL of resazurin was added. After a 2 hour incubation, fluorescence measurements were taken (excitation 535 run, emission 590 nm) and toxic effects in treated versus control cells were compared based on the degree of fluorescence in each well. Compounds E, F, O and Q were found to be cytotoxic at concentrations less than 75 μg/mL and compounds A, C, D, G, H, I, J, K, L, M, N and P were found to have minimal cytotoxicity at 75 μg/mL.

Phototoxicity Assay

Mouse 3T3 fibroblast cells were harvested and plated at a concentration of 1×10⁵ cells/mL. Drug dilutions were made in HBSS and added to the plates. Duplicate plates were then incubated in the dark (for controls), or under UV light (meter reading of 1.6-1.8 mW/cm²) for 50 minutes. Cells were then washed with HBSS, fresh medium was added, and plates were then incubated overnight. The following day, neutral red was added as an indicator of cell viability and plates were incubated for an additional 3 hours. Cells were then washed with HBSS and a solution of 50% EtOH, 10% glacial acetic acid is added. After a 20 minute incubation period, plates absorbance at 535 nm were read using a Wallac Victor 5 spectrophotometer. The phototoxicity in light-treated and control cultures was then compared. When incubated in the dark, Compounds O, P and Q were found to be phototoxic at concentrations less than 150 μM and compounds A, C, D, E, F, G, H, I, J and M were found to be not to be appreciably phototoxic at a concentration of 150 μM. When incubated in the UV light, compounds A, C, I, E, F, H, M, O and Q were found to be phototoxic at concentrations less than 30 μM and compounds D, G, I, M and P were found to be minimally phototoxic at a concentration of 30 μM.

Equivalents

Those skilled in the art will recognize, or be able to ascertain using no more than routine experimentation, numerous equivalents to the specific procedures described herein. Such equivalents are considered to be within the scope of the present invention and are covered by the following claims. The contents of all references, patents, and patent applications cited throughout this application are hereby incorporated by reference. The appropriate components, processes, and methods of those patents, applications and other documents may be selected for the present invention and embodiments thereof.

1. A tetracycline compound of the formula (I): 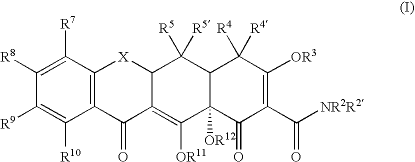
wherein
X is CHC(R¹³Y′Y), C═CR¹³Y, CR^{6′}R⁶, S, NR⁶, or O;
R^{2′}, R^{2″}, R^{4a}, and R^{4b} are each independently hydrogen, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R³, R¹¹ and R¹² are each are each independently hydrogen, alkyl, alkenyl, aryl, alkynyl, aralkyl, acetyl, alkylcarbonyl, alkenylcarbonyl, arylcarbonyl, alkynylcarbonyl, alkyloxycarbonyl, alkenyloxycarbonyl, alkynyloxycarbonyl, aryloxycarbonyl, alkylaminocarbonyl, alkenylaminocarbonyl, alkynylaminocarbonyl, arylaminocarbonyl, alkylthiocarbonyl, alkenylthiocarbonyl, alkynylthiocarbonyl, arylthiocarbonyl, alkyloxythiocarbonyl, alkenyloxythiocarbonyl, alkynyloxythiocarbonyl, aryloxythiocarbonyl, alkylaminothiocarbonyl, alkenylaminothiocarbonyl, alkynylaminothiocarbonyl, arylaminothiocarbonyl, alkylthiothiocarbonyl, alkenylthiothiocarbonyl, alkynylthiothiocarbonyl, or arylthiothiocarbonyl;
R⁴ and R^{4′} are each independently NR^{4a}R^{4b}, alkyl, alkenyl, alkynyl, hydroxyl, halogen, or hydrogen;
R⁵ and R^{5′} are each independently hydroxyl, hydrogen, thiol, alkanoyl, aroyl, alkaroyl, aryl, heteroaromatic, alkyl, alkenyl, alkynyl, alkoxy, alkylthio, alkylsulfinyl, alkylsulfonyl, alkylamino, arylalkyl, alkyl carbonyloxy, or aryl carbonyloxy;
R⁶ and R^{6′} are each independently hydrogen, methylene, absent, hydroxyl, halogen, thiol, alkyl, alkenyl, alkynyl, aryl, alkoxy, alkylthio, alkylsulfinyl, alkylsulfonyl, alkylamino, or an arylalkyl;
R⁷ is hydrogen, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino, acylamino, amidino, imino, sulfhydryl, alkylthio, arylthio, thiocarboxylate, sulfates, alkylsulfinyl, arylsulfinyl, alkylsulfonyl, arylsulfonyl, sulfonato, sulfamoyl, sulfonamido, nitro, trifluoromethyl, cyano, azido, alkylaryl, aryl, a heterocyclic moiety or —(CH₂)₀₋₃(NR^{7c})₀₋₁C(═W′)WR^{7a};
R⁸ is hydrogen, hydroxyl, halogen, thiol, nitro, alkyl, alkenyl, alkynyl, aryl, alkoxy, alkylthio, alkylsulfinyl, alkylsulfonyl, alkylamino, amino, arylalkenyl, arylalkynyl, acyl, aminoalkyl, heterocyclic, thionitroso, or —(CH₂)₀₋₃(NR^{8c})₀₋₁C(=E′)ER^{8a};
R⁹ is hydrogen, hydroxyl, halogen, thiol, nitro, alkyl, alkenyl, alkynyl, aryl, alkoxy, alkylthio, alkylsulfinyl, alkylsulfonyl, arylalkyl, amido, carboxylate, aminocarbonyl, arylalkenyl, arylalkynyl, acyl, aminoalkyl, heterocyclic, thionitroso, or —(CH₂)₀₋₃(NR^{9c})₀₋₁C(=Z′)ZR^{9a};
R¹⁰ is hydrogen, alkyl, alkenyl, alkynyl, halogen, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, arylaminocarbonyl, alkylthiocarbonyl, arylthiocarbonyl; phosphate, phosphonato, phosphinato, cyano, amino, acylamino, amidino, imino, sulfhydryl, alkylthio, arylthio, thiocarboxylate, sulfates, alkylsulfinyl, arylsulfinyl, alkylsulfonyl, arylsulfonyl, sulfonato, sulfamoyl, sulfonamido, nitro, trifluoromethyl, cyano, azido, alkylaryl, aryl or a heterocyclic moiety;
R^{7a}, R^{7b}, R^{7c}, R^{7d}, R^{7e}, R^{7f}, R^{8a}, R^{8b}, R^{8c}, R^{8d}, R^{8e}, R^{8f}, R^{9a}, R^{9b}, R^{9c}, R^{9d}, R^{9e}, and R^{9f} are each independently hydrogen, acyl, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R¹³ is hydrogen, hydroxy, alkyl, alkenyl, alkynyl, alkoxy, alkylthio, aryl, alkylsulfinyl, alkylsulfonyl, alkylamino, or an arylalkyl;
E is CR^{8d}R^{8e}, S, NR^{8b} or O;
E′ is O, NR^{8f}, or S;
W is CR^{7d}R^{7e}, S, NR^{7b} or O;
W′ is O, NR^{7f}, or S;
Y′ and Y are each independently hydrogen, halogen, hydroxyl, cyano, sulfhydryl, amino, alkyl, alkenyl, alkynyl, alkoxy, alkylthio, alkylsulfinyl, alkylsulfonyl, alkylamino, or an arylalkyl;
Z is CR^{9d}R^{9e}, S, NR^{9b} or O;
Z′ is O, S, or NR^{9f}, and pharmaceutically acceptable salts, esters and enantiomers thereof.

2. The tetracycline compound of claim 1, wherein R², R^{2′}, R³, R¹¹, and R¹² are each hydrogen or a prodrug moiety; R⁴ is NR^{4a}R^{4b}; R^{4a} and R^{4b} are each alkyl; X is CR⁶R^{6′}; and R^{4′}, R⁵, R^{5′}, R⁶, and R^{6′} are each hydrogen.

3. The tetracycline compound of claim 1, wherein R², R^{2′}, R³, R¹¹, and R¹² are each hydrogen or a prodrug moiety; R⁴ is NR^{4a}R^{4b}; R^{4a} l and R^{4b} are each alkyl; R^{4′}, R⁵ and R^{5′} are hydrogen and X is CR⁶R^{6′}, wherein R⁶ is methyl and R^{6′} is hydroxy.

4. The tetracycline compound of claim 1, wherein R², R^{2′}, R³, R¹¹, and R¹² are each hydrogen or a prodrug moiety; R⁴ is NR^{4a}R^{4b}; R^{4a} and R^{4b} are each alkyl; R⁵ is hydroxyl; X is CR⁶R^{6′}; R⁶ is methyl; and R^{4′}, R^{5′} and R^{6′} are hydrogen.

5. The tetracycline compound of claim 1, wherein R², R^{2′}, R³, R¹¹, and R¹² are each hydrogen or a prodrug moiety; R⁴ is NR^{4a}R^{4b}; R^{4a} and R^{4b} are each alkyl; X is CR⁶R^{6′}; R^{4′}, R⁵, R^{5′}, R⁶ and R^{6′} are hydrogen atoms and R⁷ is dimethylamino.

6. The tetracycline compound of claim 1, wherein R¹⁰ is hydrogen.

7. The tetracycline compound of claim 1, wherein R¹⁰ is alkylsulfonyl.

8. The tetracycline compound of claim 7, wherein said alkylsulfonyl is trifluoromethylsulfonyl.

9. The tetracycline compound of claim 1, wherein R¹⁰ is substituted or unsubstituted amino.

10. The tetracycline compound of claim 8, wherein R¹⁰ is dialkylamino.

11. The tetracycline compound of claim 8, wherein R¹⁰ is substituted or unsubstituted piperidinyl.

12. The tetracycline compound of claim 10, wherein R¹⁰ is 4-methylpiperidine.

13. The tetracycline compound of claim 1, wherein R⁹ is hydrogen.

14. The tetracycline compound of claim 1, wherein R⁹ is substituted or unsubstituted aryl.

15. The tetracycline compound of claim 1, wherein R⁹ is substituted or unsubstituted phenyl.

16. The tetracycline compound of claim 1, wherein R⁹ is substituted or unsubstituted alkyl.

17. The tetracycline compound of claim 16, wherein R⁹ is aminoalkyl.

18. The tetracycline compound of claim 17, wherein R⁹ is aminomethyl.

19. The tetracycline compound of claim 18, wherein R⁹ is alkylaminomethyl.

20. The tetracycline compound of claim 1, wherein R⁹ is substituted or unsubstituted amino.

21. The tetracycline compound of claim 1, wherein R⁹ is nitro or halogen.

22. The tetracycline compound of claim 21, wherein the halogen is iodine.

23. The tetracycline compound of claim 1, wherein R⁹ is substituted or unsubstituted aminocarbonyl.

24. The tetracycline compound of clam 23, wherein R⁹ is unsubstituted aminocarbonyl.

25. The tetracycline compound of claim 1, wherein R⁹ is substituted or unsubstituted carbonyl.

26. The tetracycline compound of claim 25, wherein said carbonyl is acyl.

27. The tetracycline compound of claim 25, wherein said carbonyl is carboxylate.

28. The tetracycline compound of claim 6, wherein R⁷ is substituted or unsubstituted acyl.

29. A tetracycline compound of formula (II): 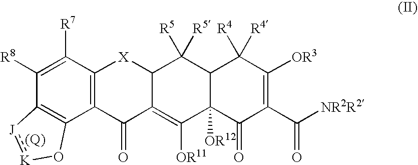
wherein
X is CHC(R¹³Y′Y), C═CR¹³Y, CR^{6′}R⁶, S, NR⁶, or O;
R^{2′}, R^{2″}, R^{4a}, and R^{4b} are each independently hydrogen, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R³, R¹¹ and R¹² are each are each independently hydrogen, alkyl, alkenyl, aryl, alkynyl, aralkyl, acetyl, alkylcarbonyl, alkenylcarbonyl, arylcarbonyl, alkynylcarbonyl, alkyloxycarbonyl, alkenyloxycarbonyl, alkynyloxycarbonyl, aryloxycarbonyl, alkylaminocarbonyl, alkenylaminocarbonyl, alkynylaminocarbonyl, arylaminocarbonyl, alkylthiocarbonyl, alkenylthiocarbonyl, alkynylthiocarbonyl, arylthiocarbonyl, alkyloxythiocarbonyl, alkenyloxythiocarbonyl, alkynyloxythiocarbonyl, aryloxythiocarbonyl, alkylaminothiocarbonyl, alkenylaminothiocarbonyl, alkynylaminothiocarbonyl, arylaminothiocarbonyl, alkylthiothiocarbonyl, alkenylthiothiocarbonyl, alkynylthiothiocarbonyl, or arylthiothiocarbonyl;
R⁴ and R^{4′} are each independently NR^{4a}R^{4b}, alkyl, alkenyl, alkynyl, hydroxyl, halogen, or hydrogen;
R⁵ and R^{5′} are each independently hydroxyl, hydrogen, thiol, alkanoyl, aroyl, alkaroyl, aryl, heteroaromatic, alkyl, alkenyl, alkynyl, alkoxy, alkylthio, alkylsulfinyl, alkylsulfonyl, alkylamino, arylalkyl, alkyl carbonyloxy, or aryl carbonyloxy;
R⁶ and R^{6′} are each independently hydrogen, methylene, absent, hydroxyl, halogen, thiol, alkyl, alkenyl, alkynyl, aryl, alkoxy, alkylthio, alkylsulfinyl, alkylsulfonyl, alkylamino, or an arylalkyl;
R⁷ is hydrogen, alkyl, alkenyl, alkynyl, halogen, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, arylaminocarbonyl, alkylthiocarbonyl, arylthiocarbonyl; phosphate, phosphonato, phosphinato, cyano, amino, acylamino, amidino, imino, sulfhydryl, alkylthio, arylthio, thiocarboxylate, sulfates, alkylsulfinyl, arylsulfinyl, alkylsulfonyl, arylsulfonyl, sulfonato, sulfamoyl, sulfonamido, nitro, trifluoromethyl, cyano, azido, alkylaryl, aryl, a heterocyclic moiety, or —(CH₂)₀₋₃(NR^{7c})₀₋₁C(═W′)WR^{7a};
R⁸ is hydrogen, hydroxyl, halogen, thiol, nitro, alkyl, alkenyl, alkynyl, aryl, alkoxy, alkylthio, alkylsulfinyl, alkylsulfonyl, alkylamino, amino, arylalkenyl, arylalkynyl, acyl, aminoalkyl, heterocyclic, thionitroso, or —(CH₂)₀₋₃(NR^{8c})₀₋₁C(=E′)ER^{8a};
R^{7a}, R^{7b}, R^{7c}, R^{7d}, R^{7e}, R^{7f}, R^{8a}, R^{8b}, R^{8c}, R^{8d}, R^{8e}, R^{8f} are each independently hydrogen, acyl, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R¹³ is hydrogen, hydroxy, alkyl, alkenyl, alkynyl, alkoxy, alkylthio, aryl, alkylsulfinyl, alkylsulfonyl, alkylamino, or an arylalkyl;
J is CR^{21a}R^{21b}, O, S, or NR^{21c};
K is CR^{22a}R^{22b}, O, S, or NR^{22c}
R^{21a}, R^{21b}, R^{21c}, R^{22a}, R^{22b}, R^{22c} are each independently hydrogen, hydroxyl, alkyl, alkenyl, alkynyl, aryl, arylalkyl, amido, alkylamino, amino, arylamino, alkylcarbonyl, arylcarbonyl, alkylaminocarbonyl, alkoxy, alkoxycarbonyl, alkylcarbonyl, alkyloxycarbonyl, arylcarbonyloxy, aryloxy, phosphate, phosphonato, phosphinato, cyano, acylamino, amidino, imino, sulfhydryl, thiol, alkylthiol, arylthiol, thiocarboxylate, sulfates, alkylsulfinyl, arylsulfinyl, alkylsulfonyl, arylsulfonyl, sulfonato, sulfamoyl, sulfonamido, nitro, trifluoromethyl, cyano, azido, heterocyclyl, alkylsilyl, arylsilyl, or absent;
Q is a double bond when J is CR^{21a}R^{21b}, K is CR^{22a}R^{22b} and R^{21b} and R^{22b} are absent;
Q is a double bond when J is NR^{21c}, K is CR^{22a}R^{22b} and R^{21c} and R^{22b} are absent;
Q is a double bond when J is CR^{21a}R^{21b}, K is NR^{22c}, and R^{21b} and R^{22c} are absent;
Q is a single bond when J is CR^{21a}R^{21b}, O, S, or NR^{21c}, K is CR^{22a}R^{22b}, O, S, or NR^{22c} and R^{21a}, R^{21b}, R^{21c}, R^{22a}, R^{22b}, R^{22c} are each independently hydrogen, hydroxyl, alkyl, alkenyl, alkynyl, aryl, arylalkyl, amido, alkylamino, amino, arylamino, alkylcarbonyl, arylcarbonyl, alkylaminocarbonyl, alkoxy, alkoxycarbonyl, alkylcarbonyl, alkyloxycarbonyl, arylcarbonyloxy, aryloxy, phosphate, phosphonato, phosphinato, cyano, acylamino, amidino, imino, sulfhydryl, thiol, alkylthiol, arylthiol, thiocarboxylate, sulfates, alkylsulfinyl, arylsulfinyl, alkylsulfonyl, arylsulfonyl, sulfonato, sulfamoyl, sulfonamido, nitro, trifluoromethyl, cyano, azido, heterocyclyl, alkylsilyl, or arylsilyl;
E is CR^{8d}R^{8e}, S, NR^{8b} or O;
E′ is O, NR^{8f}, or S;
W is CR^{7d}R^{7e}, S, NR^{7b} or O;
W′ is O, NR^{7f}, or S;
Y′ and Y are each independently hydrogen, halogen, hydroxyl, cyano, sulfhydryl, amino, alkyl, alkenyl, alkynyl, alkoxy, alkylthio, alkylsulfinyl, alkylsulfonyl, alkylamino, or an arylalkyl; and pharmaceutically acceptable salts, esters and enantiomers thereof.

30-42. (canceled)

43. A tetracycline compound of the formula (III): 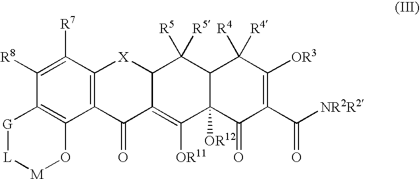
wherein
X is CHC(R¹³Y′Y), C═CR¹³Y, CR^{6′}R⁶, S, NR⁶, or O;
R^{2′}, R^{2″}, R^{4a}, and R^{4b} are each independently hydrogen, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R³, R¹¹ and R¹² are each are each independently hydrogen, alkyl, alkenyl, aryl, alkynyl, aralkyl, acetyl, alkylcarbonyl, alkenylcarbonyl, arylcarbonyl, alkynylcarbonyl, alkyloxycarbonyl, alkenyloxycarbonyl, alkynyloxycarbonyl, aryloxycarbonyl, alkylaminocarbonyl, alkenylaminocarbonyl, alkynylaminocarbonyl, arylaminocarbonyl, alkylthiocarbonyl, alkenylthiocarbonyl, alkynylthiocarbonyl, arylthiocarbonyl, alkyloxythiocarbonyl, alkenyloxythiocarbonyl, alkynyloxythiocarbonyl, aryloxythiocarbonyl, alkylaminothiocarbonyl, alkenylaninothiocarbonyl, alkynylaminothiocarbonyl, arylaminothiocarbonyl, alkylthiothiocarbonyl, alkenylthiothiocarbonyl, alkynylthiothiocarbonyl, or arylthiothiocarbonyl;
R⁴ and R^{4′} are each independently NR^{4a}R^{4b}, alkyl, alkenyl, alkynyl, hydroxyl, halogen, or hydrogen;
R⁵ and R^{5′} are each independently hydroxyl, hydrogen, thiol, alkanoyl, aroyl, alkaroyl, aryl, heteroaromatic, alkyl, alkenyl, alkynyl, alkoxy, alkylthio, alkylsulfinyl, alkylsulfonyl, alkylamino, arylalkyl, alkyl carbonyloxy, or aryl carbonyloxy;
R⁶ and R^{6′} are each independently hydrogen, methylene, absent, hydroxyl, halogen, thiol, alkyl, alkenyl, alkynyl, aryl, alkoxy, alkylthio, alkylsulfinyl, alkylsulfonyl, alkylamino, or an arylalkyl;
R⁷ is hydrogen, hydroxyl, halogen, thiol, nitro, alkyl, alkenyl, alkynyl, aryl, alkoxy, alkylthio, alkylsulfinyl, alkylsulfonyl, arylalkyl, amino, arylalkenyl, arylalkynyl, acyl, aminoalkyl, heterocyclic, thionitroso, or —(CH₂)₀₋₃(NR^{7c})₀₋₁C(═W′)WR^{7a};
R⁸ is hydrogen, hydroxyl, halogen, thiol, nitro, alkyl, alkenyl, alkynyl, aryl, alkoxy, alkylthio, alkylsulfinyl, alkylsulfonyl, alkylamino, amino, arylalkenyl, arylalkynyl, acyl, aminoalkyl, heterocyclic, thionitroso, or —(CH₂)₀₋₃(NR^{8c})₀₋₁C(=E′)ER^{8a};
R^{7a}, R^{7b}, R^{7c}, R^{7d}, R^{7e}, R^{7f}, R^{8a}, R^{8b}, R^{8c}, R^{8d}, R^{8e}, R^{8f} are each independently hydrogen, acyl, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R¹³ is hydrogen, hydroxy, alkyl, alkenyl, alkynyl, alkoxy, alkylthio, aryl, alkylsulfinyl, alkylsulfonyl, alkylamino, or an arylalkyl;
G is CR^{23a}R^{23b}, O, S, or NR^{23c};
L is CR^{24a}R^{24b}, O, S, or NR^{24c};
M is CR^{25a}R^{25b}, C=T, O, S, or NR^{25c};
T is O, S or NR^{25d};
R^{23a}, R^{23b}, R^{23c}, R^{24a}, R^{24b}, R^{24c}, R^{25a}, R^{25b}, R^{25c}, R^{25d} are each independently hydrogen, hydroxyl, alkyl, alkenyl, alkynyl, aryl, arylalkyl, amido, alkylamino, amino, arylamino, alkylcarbonyl, arylcarbonyl, alkylaminocarbonyl, alkoxy, alkoxycarbonyl, alkylcarbonyl, alkyloxycarbonyl, arylcarbonyloxy, aryloxy, phosphate, phosphonato, phosphinato, cyano, acylamino, amidino, imino, sulfhydryl, thiol, alkylthiol, arylthiol, thiocarboxylate, sulfates, alkylsulfinyl, arylsulfinyl, alkylsulfonyl, arylsulfonyl, sulfonato, sulfamoyl, sulfonamido, nitro, trifluoromethyl, cyano, azido, heterocyclyl, alkylsilyl, or arylsilyl;
E is CR^{8d}R^{8e}, S, NR^{8b} or O;
E′ is O, NR^{8f}, or S;
W is CR^{7d}R^{7e}S, NR^{7b} or O;
W′ is O, NR^{7f}, or S;
Y′ and Y are each independently hydrogen, halogen, hydroxyl, cyano, sulfhydryl, amino, alkyl, alkenyl, alkynyl, alkoxy, alkylthio, alkylsulfinyl, alkylsulfonyl, alkylamino, or an arylalkyl; and pharmaceutically acceptable salts, esters and enantiomers thereof.

44-74. (canceled)

75. The tetracycline compound of claim 1, wherein said compound is a compound of Table 1.

76. A tetracycline compound of formula (IV): 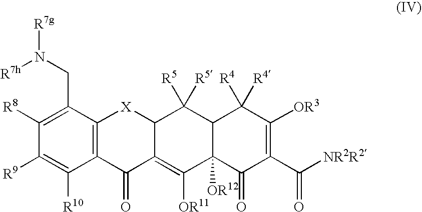
wherein
X is CHC(R¹³Y′Y), C═CR¹³Y, CR^{6′}R⁶, S, NR⁶, or O;
R^{2′}, R^{2″}, R^{4a}, and R^{4b} are each independently hydrogen, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R³, R¹¹ and R¹² are each are each independently hydrogen, alkyl, alkenyl, aryl, alkynyl, aralkyl, acetyl, alkylcarbonyl, alkenylcarbonyl, arylcarbonyl, alkynylcarbonyl, alkyloxycarbonyl, alkenyloxycarbonyl, alkynyloxycarbonyl, aryloxycarbonyl, alkylaminocarbonyl, alkenylaminocarbonyl, alkynylaminocarbonyl, arylaminocarbonyl, alkylthiocarbonyl, alkenylthiocarbonyl, alkynylthiocarbonyl, arylthiocarbonyl, alkyloxythiocarbonyl, alkenyloxythiocarbonyl, alkynyloxythiocarbonyl, aryloxythiocarbonyl, alkylaminothiocarbonyl, alkenylaminothiocarbonyl, alkynylaminothiocarbonyl, arylaminothiocarbonyl, alkylthiothiocarbonyl, alkenylthiothiocarbonyl, alkynylthiothiocarbonyl, or arylthiothiocarbonyl;
R⁴ and R^{4′} are each independently NR^{4a}R^{4b}, alkyl, alkenyl, alkynyl, hydroxyl, halogen, or hydrogen;
R⁵ and R^{5′} are each independently hydroxyl, hydrogen, thiol, alkanoyl, aroyl, alkaroyl, aryl, heteroaromatic, alkyl, alkenyl, alkynyl, alkoxy, alkylthio, alkylsulfinyl, alkylsulfonyl, alkylamino, arylalkyl, alkyl carbonyloxy, or aryl carbonyloxy;
R⁶ and R^{6′} are each independently hydrogen, methylene, absent, hydroxyl, halogen, thiol, alkyl, alkenyl, alkynyl, aryl, alkoxy, alkylthio, alkylsulfinyl, alkylsulfonyl, alkylamino, or an arylalkyl;
R^{7g} and R^{7h} are each independently hydrogen, alkyl, alkenyl, aryl, alkynyl, aralkyl, acetyl, alkylcarbonyl, alkenylcarbonyl, arylcarbonyl, alkynylcarbonyl, alkyloxycarbonyl, alkenyloxycarbonyl, alkynyloxycarbonyl, aryloxycarbonyl, alkylaminocarbonyl, alkenylaminocarbonyl, alkynylaminocarbonyl, arylaminocarbonyl, alkylthiocarbonyl, alkenylthiocarbonyl, alkynylthiocarbonyl, arylthiocarbonyl, alkyloxythiocarbonyl, alkenyloxythiocarbonyl, alkynyloxythiocarbonyl, aryloxythiocarbonyl, alkylaminothiocarbonyl, alkenylaminothiocarbonyl, alkynylaminothiocarbonyl, arylaminothiocarbonyl, alkylthiothiocarbonyl, alkenylthiothiocarbonyl, alkynylthiothiocarbonyl, arylthiothiocarbony, or R^{7g} and R^{7h} are linked together to form a ring;
R⁸ is hydrogen, hydroxyl, halogen, thiol, nitro, alkyl, alkenyl, alkynyl, aryl, alkoxy, alkylthio, alkylsulfinyl, alkylsulfonyl, alkylamino, amino, arylalkenyl, arylalkynyl, acyl, aminoalkyl, heterocyclic, thionitroso, or —(CH₂)₀₋₃(NR^{8c})₀₋₁C(=E′)ER^{8a};
E is CR^{8d}R^{8e}, S, NR^{8b} or O;
E′ is O, NR^{8f}, or S;
R⁹ is hydrogen, hydroxyl, halogen, thiol, nitro, alkyl, alkenyl, alkynyl, aryl, alkoxy, alkylthio, alkylsulfinyl, alkylsulfonyl, arylalkyl, amido, carboxylate, aminocarbonyl, arylalkenyl, arylalkynyl, acyl, aminoalkyl, heterocyclic, thionitroso, or —(CH₂)₀₋₃(NR^{9c})₀₋₁C(=Z′)ZR^{9a};
Z is CR^{9d}R^{9e}, S, NR^{9b} or O;
Z′ is O, S, or NR^{9f};
R^{8a}, R^{8b}, R^{8c}, R^{8d}, R^{8e}, R^{8f}, R^{9a}, R^{9b}, R^{9c}, R^{9d}, R^{9e}, and R^{9f} are each independently hydrogen, acyl, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R¹⁰ is hydrogen;
R¹³ is hydrogen, hydroxy, alkyl, alkenyl, alkynyl, alkoxy, alkylthio, aryl, alkylsulfinyl, alkylsulfonyl, alkylamino, or an arylalkyl;
Y′ and Y are each independently hydrogen, halogen, hydroxyl, cyano, sulfhydryl, amino, alkyl, alkenyl, alkynyl, alkoxy, alkylthio, alkylsulfinyl, alkylsulfonyl, alkylamino, or an arylalkyl;
and pharmaceutically acceptable salts, esters and enantiomers thereof.

77-78. (canceled)

79. A tetracycline compound of formula (V): 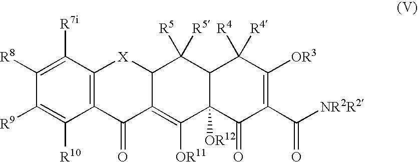
wherein
X is CHC(R¹³Y′Y), C═CR¹³Y, CR^{6′}R⁶, S, NR⁶, or O;
R^{2′}, R^{2″}, R^{4a}, and R^{4b} are each independently hydrogen, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R³, R¹¹ and R¹² are each are each independently hydrogen, alkyl, alkenyl, aryl, alkynyl, aralkyl, acetyl, alkylcarbonyl, alkenylcarbonyl, arylcarbonyl, alkynylcarbonyl, alkyloxycarbonyl, alkenyloxycarbonyl, alkynyloxycarbonyl, aryloxycarbonyl, alkylaminocarbonyl, alkenylaminocarbonyl, alkynylaminocarbonyl, arylaminocarbonyl, alkylthiocarbonyl, alkenylthiocarbonyl, alkynylthiocarbonyl, arylthiocarbonyl, alkyloxythiocarbonyl, alkenyloxythiocarbonyl, alkynyloxythiocarbonyl, aryloxythiocarbonyl, alkylaminothiocarbonyl, alkenylaminothiocarbonyl, alkynylaminothiocarbonyl, arylaminothiocarbonyl, alkylthiothiocarbonyl, alkenylthiothiocarbonyl, alkynylthiothiocarbonyl, or arylthiothiocarbonyl;
R⁴ and R^{4′} are each independently NR^{4a}R^{4b}, alkyl, alkenyl, alkynyl, hydroxyl, halogen, or hydrogen;
R⁵ and R^{5′} are each independently hydroxyl, hydrogen, thiol, alkanoyl, aroyl, alkaroyl, aryl, heteroaromatic, alkyl, alkenyl, alkynyl, alkoxy, alkylthio, alkylsulfinyl, alkylsulfonyl, alkylamino, arylalkyl, alkyl carbonyloxy, or aryl carbonyloxy;
R⁶ and R^{6′} are each independently hydrogen, methylene, absent, hydroxyl, halogen, thiol, alkyl, alkenyl, alkynyl, aryl, alkoxy, alkylthio, alkylsulfinyl, alkylsulfonyl, alkylamino, or an arylalkyl;
R⁷ⁱ is a substituted or unsubstituted heterocycle selected from the group consisting of thiophene, pyrrole, 1,3-oxazole, 1,3-thiazole, 1,3,4-oxadiazole, 1,3,4-thiadiazole, 1,2,3-oxadiazole, 1,2,3-thiadiazole, 1H-1,2,3-triazole, isothiazole, 1,2,4-oxadiazole, 1,2,4-thiadiazole, 1,2,3,4-oxatriazole, 1,2,3,4-thiatriazole, 1H-1,2,3,4-tetraazole, 1,2,3,5-oxatriazole, 1,2,3,5-thiatriazole, furan, imidazol-1-yl, imidazol-4-yl, 1,2,4-triazol-4-yl, 1,2,4-triazol-5-yl, isoxazol-3-yl, isoxazol-5-yl, pyrazol-3-yl, pyrazol-5-yl, thiolane, pyrrolidine, tetrahydrofuran, 4,5-dihydrothiophene, 2-pyrroline, 4,5-dihydrofuran, pyridazine, pyrimidine, pyrazine, 1,2,3-triazine, 1,2,4-triazine, 1,2,4-triazine, 1,3,5-triazine, pyridine, 2H-3,4,5,6-tetrahydropyran, thiane, 1,2-diazaperhydroine, 1,3-diazaperhydroine, piperazine, 1,3-oxazaperhydroine, morpholine, 1,3-thiazaperhydroine, 1,4-thiazaperhydroine, piperidine, 2H-3,4-dihydropyran, 2,3-dihydro-4H-thiin, 1,4,5,6-tetrahydropyridine, 2H-5,6-dihydropyran, 2,3-dihydro-6H-thiin, 1,2,5,6-tetrahydropyridine, 3,4,5,6-tetrahydropyridine, 4H-pyran, 4H-thiin, 1,4-dihydropyridine, 1,4-dithiane, 1,4-dioxane, 1,4-oxathiane, 1,2-oxazolidine, 1,2-thiazolidine, pyrazolidine, 1,3-oxazolidine, 1,3-thiazolidine, imidazolidine, 1,2,4-oxadiazolidine, 1,3,4-oxadiazolidine, 1,2,4-thiadiazolidine, 1,3,4-thiadiazolidine, 1,2,4-triazolidine, 2-imidazoline, 3-imidazoline, 2-pyrazoline, 4-imidazoline, 2,3-dihydroisothiazole, 4,5-dihydroisoxazole, 4,5-dihydroisothiazole, 2,5-dihydroisoxazole, 2,5-dihydroisothiazole, 2,3-dihydroisoxazole, 4,5-dihydrooxazole, 2,3-dihydrooxazole, 2,5-dihydrooxazole, 4,5-dihydrothiazole, 2,3-dihydrothiazole, 2,5-dihydrothiazole, 1,3,4-oxathiazolidine, 1,4,2-oxathiazolidine, 2,3-dihydro-1H-[1,2,3]triazole, 2,5-dihydro-1H-[1,2,3]triazole, 4,5-dihydro-1H-[1,2,3]triazole, 2,3-dihydro-1H-[1,2,4]triazole, 4,5-dihydro-1H-[1,2,4]triazole, 2,3-dihydro-[1,2,4]oxadiazole, 2,5-dihydro-[1,2,4]oxadiazole, 4,5-dihydro-[1,2,4]thiadiazole, 2,3-dihydro-[1,2,4]thidiazole, 2,5-dihydro-[1,2,4]thiadiazole, 4,5-dihydro-[1,2,4]thiadiazole, 2,5-dihydro-[1,2,4]oxadiazole, 2,3-dihydro-[1,2,4]oxadiazole, 4,5-dihydro-[1,2,4]oxadiazole, 2,5-dihydro-[1,2,4]thiadiazole, 2,3-dihydro-[1,2,4]thiadiazole, 4,5-dihydro-[1,2,4]thiadiazole, 2,3-dihydro-[1,3,4]oxadiazole, 2,3-dihydro-[1,3,4]thiadiazole, [1,4,2]oxathiazole, [1,3,4]oxathiazole, 1,3,5-triazaperhydroine, 1,2,4-triazaperhydroine, 1,4,2-dithiazaperhydroine, 1,4,2-dioxazaperhydroine, 1,3,5-oxadiazaperhydroine, 1,2,5-oxadiazaperhydroine, 1,3,4-thiadiazaperhydroine, 1,3,5-thiadiazaperhydroine, 1,2,5-thiadiazaperhydroine, 1,3,4-oxadiazaperhydroine, 1,4,3-oxathiazaperhydroine, 1,4,2-oxathiazaperhydroine, 1,4,5,6-tetrahydropyridazine, 1,2,3,4-tetrahydropyridazine, 1,2,3,6-tetrahydropyridazine, 1,2,5,6-tetrahydropyrimidine, 1,2,3,4-tetrahydropyrimidine, 1,4,5,6-tetrahydropyrimidine, 1,2,3,6-tetrahydropyrazine, 1,2,3,4-tetrahydropyrazine, 5,6-dihydro-4H-[1,2]oxazine, 5,6-dihydro-2H-[1,2]oxazine, 3,6-dihydro-2H-[1,2]oxazine, 3,4-dihydro-2H-[1,2]oxazine, 5,6-dihydro-4H-[1,2]thiazine, 5,6-dihydro-2H-[1,2]thiazine, 3,6-dihydro-2H-[1,2]thiazine, 3,4-dihydro-2H-[1,2]thiazine, 5,6-dihydro-2H-[1,3]oxazine, 5,6-dihydro-4H-[1,3]oxazine, 3,6-dihydro-2H-[1,3]oxazine, 3,4-dihydro-2H-[1,3]oxazine, 3,6-dihydro-2H-[1,4]oxazine, 3,4-dihydro-2H-[1,4]oxazine, 5,6-dihydro-2H-[1,3]thiazine, 5,6-dihydro-4H-[1,3]thiazine, 3,6-dihydro-2H-[1,3]thiazine, 3,4-dihydro-2H-[1,3]thiazine, 3,6-dihydro-2H-[1,4]thiazine, 3,4-dihydro-2H-[1,4]thiazine, 1,2,3,6-tetrahydro-[1,2,4]triazine, 1,2,3,4-tetrahydro-[1,2,4]triazine, 1,2,3,4-tetrahydro-[1,3,5]triazine, 2,3,4,5-tetrahydro-[1,2,4]triazine, 1,4,5,6-tetrahydro-[1,2,4]triazine, 5,6-dihydro-[1,4,2]dioxazine, 5,6-dihydro-[1,4,2]dioxazine, 5,6-dihydro-[1,4,2]dithiazine, 2,3-dihydro-[1,4,2]dioxazine, 3,4-dihydro-2H-[1,3,4]oxadiazine, 3,6-dihydro-2H-[1,3,4]oxadiazine, 3,4-dihydro-2H-[1,3,5]oxadiazine, 3,6-dihydro-2H-[1,3,5]oxadiazine, 5,6-dihydro-2H-[1,2,5]oxadiazine, 5,6-dihydro-4H-[1,2,5]oxadiazine, 3,4-dihydro-2H-[1,3,4]thiadiazine, 3,6-dihydro-2H-[1,3,4]thiadiazine, 3,4-dihydro-2H-[1,3,5]thiadiazine, 3,6-dihydro-2H-[1,3,5]thiadiazine, 5,6-dihydro-2H-[1,2,5]thiadiazine, 5,6-dihydro-4H-[1,2,5]thiadiazine, 5,6-dihydro-2H-[1,2,3]oxadiazine, 3,6-dihydro-2H-[1,2,5]oxadiazine, 5,6-dihydro-4H-[1,3,4]oxadiazine, 3,4-dihydro-2H-[1,2,5]oxadiazine, 5,6-dihydro-2H-[1,2,3]thiadiazine, 3,6-dihydro-2H-[1,2,5]thiadiazine, 5,6-dihydro-4H-[1,3,4]thiadiazine, 3,4-dihydro-2H-[1,2,5]thiadiazine, 5,6-dihydro-[1,4,3]oxathiazine, 5,6-dihydro-[1,4,2]oxathiazine, 2,3-dihydro-[1,4,3]oxathiazine, 2,3-dihydro-[1,4,2]oxathiazine, 4,5-dihydropyridine, 1,6-dihydropyridine, 5,6-dihydropyridine, 2H-pyran, 2H-thiin, 3,6-dihydropyridine, 2,3-dihydropyridazine, 2,5-dihydropyridazine, 4,5-dihydropyridazine, 1,2-dihydropyridazine, 2,3-dihydropyrimidine, 2,5-dihydropyrimidine, 5,6-dihydropyrimidine, 3,6-dihydropyrimidine, 4,5-dihydropyrazine, 5,6-dihydropyrazine, 3,6-dihydropyrazine, 4,5-dihydropyrazine, 1,4-dihydropyrazine, 1,4-dithiin, 1,4-dioxin, 2H-1,2-oxazine, 6H-1,2-oxazine, 4H-1,2-oxazine, 2H-1,3-oxazine, 4H-1,3-oxazine, 6H-1,3-oxazine, 2H-1,4-oxazine, 4H-1,4-oxazine, 2H-1,3-thiazine, 2H-1,4-thiazine, 4H-1,2-thiazine, 6H-1,3-thiazine, 4H-1,4-thiazine, 2H-1,2-thiazine, 6H-1,2-thiazine, 1,4-oxathiin, 2H,5H-1,2,3-triazine, 1H,4H-1,2,3-triazine, 4,5-dihydro-1,2,3-triazine, 1H,6H-1,2,3-triazine, 1,2-dihydro-1,2,3-triazine, 2,3-dihydro-1,2,4-triazine, 3H,6H-1,2,4-triazine, 1H,6H-1,2,4-triazine, 3,4-dihydro-1,2,4-triazine, 1H,4H-1,2,4-triazine, 5,6-dihydro-1,2,4-triazine, 4,5-dihydro-1,2,4-triazine, 2H,5H-1,2,4-triazine, 1,2-dihydro-1,2,4-triazine, 1H,4H-1,3,5-triazine, 1,2-dihydro-1,3,5-triazine, 1,4,2-dithiazine, 1,4,2-dioxazine, 2H-1,3,4-oxadiazine, 2H-1,3,5-oxadiazine, 6H-1,2,5-oxadiazine, 4H-1,3,4-oxadiazine, 4H-1,3,5-oxadiazine, 4H-1,2,5-oxadiazine, 2H-1,3,5-thiadiazine, 6H-1,2,5-thiadiazine, 4H-1,3,4-thiadiazine, 4H-1,3,5-thiadiazine, 4H-1,2,5-thiadiazine, 2H-1,3,4-thiadiazine, 6H-1,3,4-thiadiazine, 6H-1,3,4-oxadiazine and 1,4,2-oxathiazine, wherein the heterocycle is optionally vicinally fused with a saturated or unsaturated 5-, 6- or 7-membered ring containing 0, 1 or 2 atoms independently selected from N, O and S;
R⁸ is hydrogen, hydroxyl, halogen, thiol, nitro, alkyl, alkenyl, alkynyl, aryl, alkoxy, alkylthio, alkylsulfinyl, alkylsulfonyl, alkylamino, amino, arylalkenyl, arylalkynyl, acyl, aminoalkyl, heterocyclic, thionitroso, or —(CH₂)₀₋₃(NR^{8c})₀₋₁C(=E′)ER^{8a};
E is CR^{8d}R^{8c}, S, NR^{8b} or O;
E′ is O, NR^{8f}, or S;
R⁹ is hydrogen, hydroxyl, halogen, thiol, nitro, alkyl, alkenyl, alkynyl, aryl, alkoxy, alkylthio, alkylsulfinyl, alkylsulfonyl, arylalkyl, amido, carboxylate, aminocarbonyl, arylalkenyl, arylalkynyl, acyl, aminoalkyl, heterocyclic, thionitroso, or —(CH₂)₀₋₃(NR^{9c})₀₋₁C(=Z′)ZR^{9a};
Z is CR^{9d}R^{9e}, S, NR^{9b} or O;
Z′ is O, S, or NR^{9f};
R^{8a}, R^{8b}, R^{8c}, R^{8d}, R^{8e}, R^{8f}, R^{9a}, R^{9b}, R^{9c}, R^{9d}, R^{9e}, and and R^{9f} are each independently hydrogen, acyl, alkyl, alkenyl, alkynyl, alkoxy, alkylthio, alkylsulfinyl, alkylsulfonyl, alkylamino, arylalkyl, aryl, heterocyclic, heteroaromatic or a prodrug moiety;
R¹⁰ is hydrogen;
R¹³ is hydrogen, hydroxy, alkyl, alkenyl, alkynyl, alkoxy, alkylthio, aryl, alkylsulfinyl, alkylsulfonyl, alkylamino, or an arylalkyl;
Y′ and Y are each independently hydrogen, halogen, hydroxyl, cyano, sulfhydryl, amino, alkyl, alkenyl, alkynyl, alkoxy, alkylthio, alkylsulfinyl, alkylsulfonyl, alkylamino, or an arylalkyl;
and pharmaceutically acceptable salts, esters and enantiomers thereof.

79. (canceled)

80. A method for treating a tetracycline responsive state in a subject, comprising administering to said subject an effective amount of a tetracycline compound of claim 1, such that said tetracycline responsive state is treated.

81. The method of claim 80, wherein said subject is a mammal.

82. The method of claim 80, wherein said subject is a human.

83-88. (canceled)

89. A pharmaceutical composition comprising an effective amount of a tetracycline compound and a pharmaceutically acceptable carrier, wherein said tetracycline compound is a compound of claim 1.

90. The pharmaceutical composition of claim 89, wherein said effective amount is effective to treat a tetracycline responsive state.
